(19) United States
(12) Patent Application Publication
(10) Pub. No.: US 2026/0217707 A1
(43) Pub. Date: Jul. 30, 2026

(54) GLP-1R AGONISTS AND USES THEREOF

(71) Applicant: Fochon Biosciences, Ltd., Chongqing (CN)

(72) Inventors: Hua XU, Chongqing (CN); Chenglin ZHOU, Chongqing (CN); Xingdong ZHAO, Chongqing (CN); Weibo WANG, Moraga, CA (US)

(73) Assignee: Fochon Biosciences, Ltd., Chongqing (CN)

(21) Appl. No.: 19/148,872

(22) PCT Filed: Jan. 16, 2024
(86) PCT No.: PCT/CN2024/072529

§ 371 Date: Jul. 17, 2025

(30) Foreign Application Priority Data
Jan. 17, 2023 (WO) PCT/CN2023/072748
Mar. 31, 2023 (WO) PCT/CN2023/085615
May 12, 2023 (WO) PCT/CN2023/093742

(51) Int. Cl. C07D 471/04; A61K 31/437; A61K 31/439; A61K 31/4985; A61K 31/5377; C07D 471/18; C07D 487/04; C07D 491/048; C07D 519/00

(52) U.S. Cl. CPC C07D 471/04; A61K 31/437; A61K 31/439; A61K 31/4985; A61K 31/5377; C07D 471/18; C07D 487/04; C07D 491/048; C07D 519/00

(57) ABSTRACT

Provided are certain GLP-1R agonists of formula (I), pharmaceutical compositions thereof, and methods of use thereof.

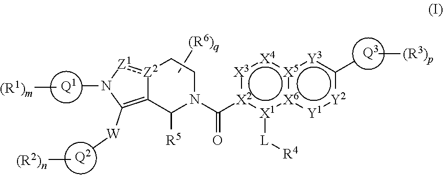

This application claims the priority to the international Patent Application No. PCT/CN2023/072748, PCT/CN2023/085615 and PCT/CN2023/093742, each of which is incorporated herein by reference in its entirety.

### FIELD OF THE INVENTION

Provided are certain compounds or pharmaceutically acceptable salts thereof which can bind to and activate Glucagon-like peptide-1 receptor (GLP-1R) and maybe useful for the treatment of metabolic disorders and related diseases, including but not limited to Type II diabetes mellitus (T2DM), obesity and non-alcoholic fatty liver disease (NASH).

### BACKGROUND OF THE INVENTION

Glucagon-like peptide-1 (GLP-1) is a peptide hormone secreted primarily by intestinal L cells after eating. GLP-1 plays a pivotal role in lowering glucose concentrations by enhancing insulin secretion and inhibiting glucagon release. Other functions of GLP-1 include delaying gastric emptying, suppressing appetite and promoting beta cell proliferation. GLP-1 effects are mediated through binding to GLP-1R which is a class B G protein-coupled receptor that is dependent on glucose. The binding of GLP-1 to GLP-1R activates heterotrimeric Gs protein, which subsequently augments adenylate cyclase activity, resulting in an increase of the intracellular cyclic adenosine monophosphate (cAMP) level, thereby enhancing glucose-stimulated insulin secretion (Pflugers Arch 1998, 435, 583-594; Basic Clin Pharmacol Toxicol 2004, 95, 252-262). GLP-1 sustains stable in the blood circulation for only 2-3 minutes owing to its short half-life, and it is inactivated by dipeptidyl peptidase 4 (DPP4) (Eur. J. Biochem 1993, 214, 829-835; Endocrinology 1995, 136, 3585-3596; Diabetes 2004, 53, 654-662).

Extensive developments of GLP-1R agonists have been conducted for the treatment of T2DM, obesity and related metabolic diseases. GLP-1R agonists can be classified as either short-acting GLP-1R agonists (exenatide and lixisenatide) or long-acting GLP-1R agonists (exenatide-LAR, liraglutide, albiglutide, and dulaglutide) according to their pharmacological properties. However, the aforementioned GLP-1R agonists are mainly administered by subcutaneously injection. The availability of oral GLP-1R agonists that can provide easier administration treatment is limited. For example, semaglutide, approved by the US FDA in 2019, is the only once-daily oral GLP-1R agonist available.

Therefore, there is an urgent need for novel GLP-1R agonists that have at least one advantageous property selected from easy administration, solubility, drug-drug interactions, potency, stability, selectivity, toxicity, drug resistance, pharmacokinetics and pharmacodynamics properties as an alternative for the treatment of metabolic disorders and related diseases, including but not limited to T2DM, obesity and NASH. In this regard, a novel class of GLP-1R agonists is provided herein.

### DISCLOSURE OF THE INVENTION

Disclosed herein are certain novel compounds, pharmaceutically acceptable salts thereof, and pharmaceutical compositions thereof, and their use as pharmaceuticals.

In one aspect, disclosed herein is a compound of formula (I),

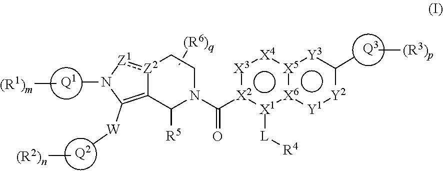

or a pharmaceutically acceptable salt thereof, wherein:
[symbol] is a double bond or a single bond;
X¹, X², X⁵ and X⁶ are independently selected from N and C;
X³ is selected from O, S, N, NR^{7.3} and CR^{7.3};
X⁴ is absent or selected from N, C(O) and CR^{7.4.}
Y¹ is selected from N and CR^{8.1};
Y² is selected from N and CR^{8.2};
Y³ is selected from N and CR^{8.3};
Z¹ is selected from N and C(O);
Z² is selected from N and C:
W is selected from

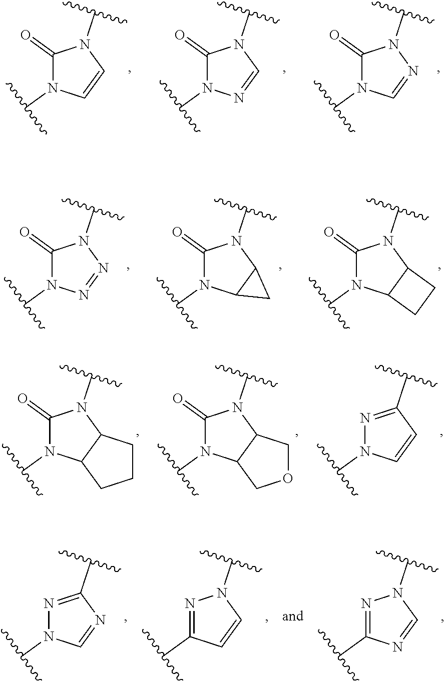

which are each unsubstituted or substituted with at least one substituent, independently selected from R^{X};
L is selected from —CR^{C0}R^{D0}(CR^{C0}R^{D0})ᵤ—, —(CR^{C0}R^{D0})ᵤO(CR^{C0}R^{D0})ₜ—, (CR^{C0}R^{D0})ᵤNR^{A0}(CR^{C0}R^{D0})ₜ— and —(CR^{C0}R^{D0})ᵤS(O), (CR^{C0}R^{D0})ₜ—;
Q¹, Q² and Q³ is selected from C₃₋₁₀ cycloalkyl, heterocyclyl, aryl and heteroaryl;
each R¹, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═NR^{E1})R^{A1}, —C(═N—OR^{B1})R^{A1}, —C(O)OR^{A1}, —OC(O)R^{A1}, —C(O)NR^{A1}R^{B1}, —C(O)NR^{A1}S(O)ᵣR^{A1}, —C(O)NR^{A1}S(O)₂OR^{A1}, —C(O)NR^{A1}S(O)ᵣNR^{A1}R^{B1}, —C(O)NR^{A1}S(O)(═NR^{E1})R^{B1}, —C(O)NR^{A1}S(O)(═NR^{E1}) NR^{A1}R^{B1}, —NR^{A1}C(O)R^{B1}, —C(═NR^{E1}) NR^{A1}R^{B1}, —NR^{A1}C(═NR^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1}) NR^{A1}R^{B1}, —S(O)ᵣR^{A1}, —S(O)(═NR^{E1})R^{B1}, —N═S(O)R^{A1}R^{B1}, —S(O)₂NR^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}. —S(O)(═NR^{E1}) NR^{A1}R^{B1}, —NR^{A1}S(O)₂NR^{A1}R^{B1}, —NR^{A1}S(O)(═NR^{E1}) NR^{A1}R^{B1}, —P(O)R^{A1}R^{B1} and —P(O)(OR^{A1})(OR^{B1}), wherein alkyl, alkenyl, alkynyl, cycloalkyl, heterocyclyl, aryl and heteroaryl are each unsubstituted or substituted with at least one substituent, independently selected from R^{X1};
each R², at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —C(O)NR^{A2}S(O)(═NR^{E2})R^{B2}, —C(O)NR^{A2}S(O)(═NR^{E2})NR^{A2}R^{B2}, —NR^{A2}C(O)R^{B2}, —C(═NR^{E2}) NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2})NR^{A2}R^{B2}, —S(O)(═NR^{E2})R^{B2}, —N═S(O)R^{A2}R^{B2}, —NR^{A2}S(O)(═NR^{E2})R^{B2}, —S(O)(═NR^{E2}) NR^{A2}R^{B2} and —NR^{A2}S(O)(═NR^{E2}) NR^{A2}R^{B2}, wherein alkyl, alkenyl, alkynyl, cycloalkyl, heterocyclyl, aryl and heteroaryl are each unsubstituted or substituted with at least one substituent, independently selected from R^{X2};
each R³, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —C(O)NR^{A3}S(O)ᵣR^{A3}, —C(O)NR^{A3}S(O)₂OR^{A3}, —C(O)NR^{A3}S(O)ᵣNR^{A3}R^{B3}, —C(O)NR^{A3}S(O)(═NR^{E3})R^{B3}, —C(O)NR^{A3}S(O)(═NR^{E3}) NR^{A3}R^{B3}, —NR^{A3}C(O)R^{B3}, —C(═NR^{E3}) NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3}) NR^{A3}R^{B3}, —S(O)ᵣR^{A3}, —S(O)(═NR^{E3})R^{B3}, —N═S(O)R^{A3}R^{B3}, —S(O)ᵣNR^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3}) NR^{A3}R^{B3}, —NR^{A3}S(O)₂NR^{A3}R^{B3}, —NR^{A3}S(O)(═NR^{E3}) NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R^{X3};
R⁴ is selected from —C(O)OH, —C(O)NHS(O)ᵣR^{A4}, —(CR^{c1}R^{d1})ₜNHS(O)ᵣR^{A4}, heterocyclyl and heteroaryl;
R⁵ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A5}R^{B5}, —OR^{A5} and —C(O)R^{A5}, wherein alkyl, alkenyl, alkynyl, cycloalkyl, and heterocyclyl are each unsubstituted or substituted with at least one substituent, independently selected from R^{X5};
each R⁶, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A6}R^{B6}, —OR^{A6} and —C(O)R^{A6}, wherein alkyl, alkenyl, alkynyl, cycloalkyl, and heterocyclyl are each unsubstituted or substituted with at least one substituent, independently selected from R^{X6};
or “R⁵ and R⁶” or “two of R⁶” together with the atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X6};
R^{7.3} and R^{7.4} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A7}R^{B7}, —OR^{A7}, —C(O)R^{A7}, —C(═NR^{E7})R^{A7}, —C(═N—OR^{B7})R^{A7}, —C(O)OR^{A7}, —OC(O)R^{A7}, —C(O)NR^{A7}R^{B7}, —C(O)NR^{A7}S(O)ᵣR^{A7}, —C(O)NR^{A7}S(O)₂OR^{A7}, —C(O)NR^{A7}S(O)ᵣNR^{A7}R^{B7}, —C(O)NR^{A7}S(O)(═NR^{E7})R^{B7}, —C(O)NR^{A7}S(O)(═NR^{E7}) NR^{A7}R^{B7}, —NR^{A7}C(O)R^{B7}, —C(═NR^{E7}) NR^{A7}R^{B7}, —NR^{A7}C(═NR^{E7})R^{B7}, —OC(O)NR^{A7}R^{B7}, —NR^{A7}C(O)OR^{B7}, —NR^{A7}C(O)NR^{A7}R^{B7}, —NR^{A7}C(S)NR^{A7}R^{B7}, —NR^{A7}C(═NR^{E7}) NR^{A7}R^{B7}, —S(O)ᵣR^{A7}, —S(O)(═NR^{E7})R^{B7}, —N═S(O)R^{A7}R^{B7}, —S(O)₂OR^{A7}, —OS(O)₂R^{A7}, —NR^{A7}S(O)ᵣR^{B7}, —NR^{A7}S(O)(═NR^{E7})R^{B7}, —S(O)ᵣNR^{A7}R^{B7}, —S(O)(═NR^{E7})NR^{A7}R^{B7}, —NR^{A7}S(O)₂NR^{A7}R^{B7}, —NR^{A7}S(O)(═NR^{E7}) NR^{A7}R^{B7}, —P(O)R^{A7}R^{B7} and —P(O)(OR^{A7})(OR^{B7}), wherein alkyl, alkenyl, alkynyl, cycloalkyl, heterocyclyl, aryl and heteroaryl are each unsubstituted or substituted with at least one substituent, independently selected from R^{X7}.
R^{8.1}, R^{8.2} and R^{8.3} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A8}R^{B8}, —OR^{A8}, —C(O)R^{A8}, —C(═NR^{E8})R^{A8}, —C(═N—OR^{B8})R^{A8}, —C(O)OR^{A8}, —OC(O)R^{A8}, —C(O)NR^{A8}R^{B8}, —C(O)NR^{A8}S(O)ᵣR^{A8}, —C(O)NR^{A8}S(O)₂OR^{A8}, —C(O)NR^{A8}S(O)ᵣNR^{A8}R^{B8}, —C(O)NR^{A8}S(O)(═NR^{E8})R^{B8}, —C(O)NR^{A8}S(O)(═NR^{E8}) NR^{A8}R^{B8}, —NR^{A8}C(O)R^{B8}, —C(═NR^{E8}) NR^{A8}R^{B8}, —NR^{A8}C(═NR^{E8})R^{B8}, —OC(O)NR^{A8}R^{B8}, —NR^{A8}C(O)OR^{B8}, —NR^{A8}C(O)NR^{A8}R^{B8}, —NR^{A8}C(S)NR^{A8}R^{B8}, —NR^{A8}C(═NR^{E8}) NR^{A8}R^{B8}, —S(O), R^{A8}, —S(O)(═NR^{E8})R^{B8}, —N═S(O)R^{A8}R^{B8}, —S(O)₂OR^{A8}, —OS(O)₂R^{A8}, —NR^{A8}S(O)ᵣR^{B8}, —NR^{A8}S(O)(═NR^{E8})R^{B8}, —S(O)ᵣNR^{A8}R^{B8}, —S(O)(═NR^{E8})NR^{A8}R^{B8}, —NR^{A8}S(O)₂NR^{A8}R^{B8}, —NR^{A8}S(O)(═NR^{E8}) NR^{A8}R^{B8}, —P(O)R^{A8}R^{B8} and —P(O)(OR^{A8})(OR^{B8}), wherein alkyl, alkenyl, alkynyl, cycloalkyl, heterocyclyl, aryl and heteroaryl are each unsubstituted or substituted with at least one substituent, independently selected from R^{X8};
R^{A0} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
each R^{A1} and R^{B1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X1};
or each “R^{A1} and R^{B1}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X1} groups;
each R^{A2} and R^{B2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X2};
or each “R^{A2} and R^{B2}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X2} groups;
each R^{A3} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X3};
or each “R^{A3} and R^{B3}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X3} groups;
R^{A4} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X4};
each R^{A5} and R^{B5} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X5};
or each “R^{A5} and R^{B5}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X5} groups;
each R^{A6} and R^{B6} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X6};
or each “R^{A6} and R^{B6}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X6} groups;
each R^{A7} and R^{B7} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X7};
or each “R^{A7} and R^{B7}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X7} groups;
each R^{A8} and R^{B8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X8};
or each “R^{A8} and R^{B8}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X8} groups;
each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
or R^{C0} and R^{D0} together with the carbon atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X0};
each R^{E1}, R^{E2}, R^{E3}, R^{E7} and R^{E8} are independently selected from hydrogen, C₁₋₁₀ alkyl, CN, NO₂, —OR^{a1}, —SR^{a1}, —S(O)ᵣR^{a1}, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from RX;
provided that when Z¹ is N, X² is C, X³ is CR^{7.3} or N, X⁴ is absent, R⁵ and R⁶ or any two of R⁶ do not form any ring, W is

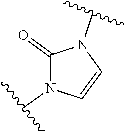 [SMILES: *n1ccn(*)c1=O]

and L is selected from —CR^{C0}R^{D0}(CR^{C0}R^{D0})ᵤ—, then at least one of R^{C0} and R^{D0} is substituted with R^{X0}, and R^{X0} is selected from CN, C₂₋₁₀ alkenyl and C₂₋₁₀ alkynyl;
each R^{X}, R^{X0}, R^{X1}, R^{X2}, R^{X3}, R^{X4}, R^{X5}, R^{X6}, R^{X7} and R^{X8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1}, —(CR^{c1}R^{d1})ₜC(═N—OR^{b1})R^{a1}, —(CR^{c1}R^{d1})ₜC(O)OR^{b1}, —(CR^{c1}R^{d1})ₜC(O)R^{b1}, —(CR^{c1}R^{d1})ₜC(O)NR^{a1}R^{b1}, —(CR^{c1}R^{d1})ₜNR^{a1}C(O)R^{b1}, —(CR^{c1}R^{d1})ₜC(═NR^{e1}) NR^{a1}R^{b1}, —(CR^{c1}R^{d1})ₜNR^{a1}C(═NR^{e1})R^{b1}, —(CR^{c1}R^{d1})ₜNR^{a1}C(O)OR^{b1}, —(CR^{c1}R^{d1})ₜNR^{a1}C(O)NR^{a1}R^{b1}, —(CR^{c1}R^{d1})ₜC(O)NR^{a1}R^{b1}, —(CR^{c1}R^{d1})ₜNR^{a1}C(S)NR^{a1}R^{b1}, —(CR^{c1}R^{d1})ₜNR^{a1}C(═NR^{e1})NR^{a1}R^{b1}, —(CR^{c1}R^{d1})S(O)ᵣR^{b1}, —(CR^{c1}R^{d1})ₜS(O)(═NR^{e1})R^{b1}, —(CR^{c1}R^{d1})ₜN═S(O)R^{a1}R^{b1}, —(CR^{c1}R^{d1})S(O)₂OR^{b1}, —(CR^{c1}R^{d1})ₜS(O)₂R^{b1}, —(CR^{c1}R^{d1})ₜOS(O)ₜNR^{a1}R^{b1}, —(CR^{c1}R^{d1})ₜNR^{a1}S(O)ᵣR^{b1}, —(CR^{c1}R^{d1})ₜNR^{a1}S(O)(═NR^{e1})R^{b1}, —(CR^{c1}R^{d1})ₜS(O)ᵣNR^{a1}R^{b1}, —(CR^{c1}R^{d1})ₜS(O)(═NR^{e1})NR^{a1}R^{b1}, —(CR^{c1}R^{d1})ₜNR^{a1}S(O)₂NR^{a1}R^{b1}, (CR^{c1}R^{d1})ₜNR^{a1}S(O)(═NR^{e1}) NR^{a1}R^{b1}, —(CR_{c1}R^{d1})ₜP(O)R^{a1}R^{b1} and —(CR^{c1}R^{d1})ₜP(O)(OR^{a1})(OR^{b1}), wherein alkyl, alkenyl, alkynyl, cycloalkyl, heterocyclyl, aryl and heteroaryl are each unsubstituted or substituted with at least one substituent, independently selected from RY;
each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from RY;
or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 RY groups;
each R^{c1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};
or R^{e1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 RY groups;
each R^{e1} is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR^{a2}R^{b2} and —C(O)NR^{a2}R^{b2};
each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})ₜNR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a2}, —(CR^{c2}R^{d2})ₜC(═N—OR^{b2})R^{a2}, —(CR^{c2}R^{d2})ₜC(O)OR^{b2}, —(CR^{c2}R^{d2})ₜC(O)R^{b2}, —(CR^{c2}R^{d2})ₜC(O)NR^{a2}R^{b2}, —(CR^{c2}R^{d2})ₜNR^{a2}C(O)R^{b2}, —(CR^{c2}R^{d2})ₜC(═NR^{e2})NR^{a2}R^{b2}, (CR^{c2}R^{d2})ₜNR^{a2}C(═NR^{e2})R^{b2}, —(CR^{c2}R^{d2})ₜOC(O)NR^{a2}R^{b2}, —(CR^{c2}R^{d2})ₜNR^{a2}C(O)OR^{b2}, (CR^{c2}R^{d2})ₜNR^{a2}C(O)NR^{a2}R^{b2}, —(CR^{c2}R^{d2})ₜNR^{a2}C(S)NR^{a2}R^{b2}, —(CR^{c2}R^{d2})ₜNR^{a2}C(═NR^{e2})NR^{a2}R^{b2}, —(CR^{c2}R^{d2})ₜS(O)ᵣR^{b2}, —(CR^{c2}R^{d2})ₜS(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2}); OS(O)₂R^{b2}, —(CR^{c2}R^{d2})ₜNR^{a2}S(O)ᵣR^{b2}, —(CR^{c2}R^{d2})ₜS(O)ᵣNR^{a2}R^{b2}, (CR^{c2}R^{d2})ₜS(O)(═NR^{e2})NR^{a2}R^{b2}, —(CR^{c2}R^{d2})ₜNR^{a2}S(O)₂NR^{a2}R^{b2}, —(CR^{c2}R^{d2})ₜNR^{a2}S(O)(═NR^{e2})NR^{a2}R^{b2}, —(CR^{c2}R^{d2})ₜP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;
each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{a2} and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{c2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{e2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O) C₁₋₄ alkyl, —C(O) C₃₋₁₀ cycloalkyl, —C(O)OC₁₋₄ alkyl, —C(O)OC₃₋₁₀ cycloalkyl, —C(O)N(C₁₋₄ alkyl)₂, —C(O)N(C₃₋₁₀ cycloalkyl)₂, —S(O)₂C₁₋₄ alkyl, —S(O)₂C₃₋₁₀ cycloalkyl, —S(O)₂N(C₁₋₄ alkyl) 2 and —S(O)₂N(C₃₋₁₀ cycloalkyl) 2;
m, n, p and q are independently selected from 0, 1, 2, 3 and 4;
each r is independently selected from 0, 1 and 2;
each t is independently selected from 0, 1, 2, 3 and 4;
each u is independently selected from 0, 1, 2, 3 and 4.

In yet another aspect, the present disclosure provides pharmaceutical compositions comprising a compound of formula (I) or at least one pharmaceutically acceptable salt thereof and a pharmaceutically acceptable excipient.

In yet another aspect, the disclosure provides methods for modulating GLP-1R, comprising administering to a system or a subject in need thereof, a therapeutically effective amount of a compound of formula (I) or a pharmaceutically acceptable salt thereof or pharmaceutical compositions thereof, thereby modulating said GLP-1R.

In yet another aspect, disclosed is a method to treat, ameliorate or prevent a condition which responds to inhibition of GLP-1R comprising administering to a system or subject in need of such treatment an effective amount of a compound of formula (I) or a pharmaceutically acceptable salt thereof or pharmaceutical compositions thereof, and optionally in combination with a second therapeutic agent, thereby treating said condition.

Alternatively, the present disclosure provides the use of a compound of formula (I) or a pharmaceutically acceptable salt thereof in the manufacture of a medicament for treating a condition mediated by GLP-1R. In particular embodiments, the compounds of the disclosure may be used alone or in combination with a second therapeutic agent to treat a condition mediated by GLP-1R.

Alternatively, disclosed is a compound of formula (I) or a pharmaceutically acceptable salt thereof for treating a condition mediated by GLP-1R.

Specifically, the condition herein includes but not limited to, an autoimmune disease, a transplantation disease, an infectious disease or a GLP-1 receptor-mediated disease or disorder. The novel class of GLP-1R inhibitors is provided herein have at least one advantageous property selected from easy administration, solubility, drug-drug interactions, potency, stability, selectivity, toxicity, drug resistance, pharmacokinetics and pharmacodynamics properties properties as an alternative for the treatment of GLP-1 receptor-mediated disease or disorder, including but not limited to T2DM, obesity and NASH.

Furthermore, the disclosure provides methods for treating a GLP-1 receptor-mediated disease or disorder, comprising administering to a system or subject in need of such treatment an effective amount of a compound of formula (I) or a pharmaceutically acceptable salt thereof or pharmaceutical compositions thereof, and optionally in combination with a second therapeutic agent, thereby treating said condition.

Alternatively, the present disclosure provides the use of a compound of formula (I) or a pharmaceutically acceptable salt thereof in the manufacture of a medicament for treating a GLP-1 receptor-mediated disease or disorder or of modulating GLP-1 receptor.

Specifically, the GLP-1 receptor-mediated disease or disorder is includes but not limited to, diabetes, diabetic complication, obesity, impaired glucose tolerance, overweight condition, hyperlipidemia, hypercholesteremia, atherosclerosis, hypertension, coronary heart disease, congestive heart failure, cardiac arrhythmias, brain infarction, stroke, liver disease, nonalcoholic fatty liver disease (NAFLD), nonalcoholic steatohepatitis (NASH), dementia, Parkinson's disease, and diabetic kidney disease.

In the above methods for using the compounds of the disclosure, a compound of formula (I) or a pharmaceutically acceptable salt thereof may be administered to a system comprising cells or tissues, or to a subject including a mammalian subject such as a human or animal subject.

### Certain Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail.

It is to be understood that the foregoing general description and the following detailed description are explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, CH₂O is equivalent to OCH₂.

The term “substituted” means that a hydrogen atom is replaced by a substituent. It is to be understood that substitution at a given atom is limited by valency. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. Generally, when any substituent or group occurs more than one time in any constituent or compound, its definition on each occurrence is independent of its definition at every other occurrence. Such combinations of substituents and/or variables, however, are permissible only if such combinations result in stable compounds.

The “ [symbol] ” symbol indicates the point of attachment to the rest of the molecule.

It is to be understood that when there are two or more variables (e.g. R¹) occur at the structure of a compound at the same time, each is selected independently. As a non-limiting example, there are two R¹ occur at the structure of a compound at the same time, one R¹ is methyl and the other R¹ is F.

The term “optional” or “optionally” means the event described subsequent thereto may or may not happen. This term encompasses the cases that the event may or may not happen.

The term “Cᵢ₋ⱼ” or “i-j membered” used herein means that the moiety has i-j carbon atoms or i-j atoms. For example, “C₁₋₆ alkyl” means said alkyl has 1-6 carbon atoms. Likewise, C₃₋₁₀ cycloalkyl means said cycloalkyl has 3-10 carbon atoms.

The term “hydrogen” refers to 1H, 2H and 3H.

It is to be understood that when there are two or more Rⁿ or R^{Xn} (n being 1, 2, 3, 4, 5, 6, 7, etc.), each Rⁿ or each R^{Xn} is selected independently.

When any variable (e.g. R) occurs at the structure of a compound over one time, it is defined independently at each case. Therefore, for example, if a group is substituted by 0-2R, the group may be optionally substituted by at most two R and R has independent option at each case. Additionally, a combination of substituents and/or the variants thereof are allowed only if such a combination will result in a stable compound.

The expression “one or more” or “at least one” refers to one, two, three, four, five, six, seven, eight, nine or more.

Unless stated otherwise, the term “hetero” means heteroatom or heteroatom radical (i.e. a radical containing heteroatom), i.e. the atoms beyond carbon and hydrogen atoms or the radical containing such atoms. Preferably, the heteroatom(s) is independently selected from the group consisting of O, N, S, P and the like. In an embodiment wherein two or more heteroatoms are involved, the two or more heteroatoms may be the same, or part or all of the two or more heteroatoms may be different.

The term “alkyl”, employed alone or in combination with other terms, refers to branched or straight-chain saturated aliphatic hydrocarbon groups having the specified number of carbon atoms. Unless otherwise specified, “alkyl” refers to C₁₋₁₀ alkyl. For example, C₁₋₆, as in “C₁₋₆ alkyl” is defined to include groups having 1, 2, 3, 4, 5, or 6 carbons in a linear or branched arrangement. For example, “C₁₈ alkyl” includes but is not limited to methyl, ethyl, n-propyl, i-propyl, n-butyl, t-butyl, i-butyl, pentyl, hexyl, heptyl, and octyl. Each “hydrogen” in the “alkyl” is independently 1H, 2H or 3H.

The term “cycloalkyl”, employed alone or in combination with other terms, refers to a saturated monocyclic or multicyclic (e.g. bicyclic or tricyclic) hydrocarbon ring system, usually with 3 to 16 ring atoms. The ring atoms of cycloalkyl are all carbon and the cycloalkyl contains zero heteroatoms and zero double bonds. In a multicyclic cycloalkyl, two or more rings can be fused or bridged or spiro together. Examples of monocyclic ring systems include but are not limited to cyclopropyl, cyclobutyl, cyclopentyl, cyclohexyl, cycloheptyl, and cyclooctyl. The bridged cycloalkyl is a polycyclic ring system containing 3-10 carbon atoms, which contains one or two alkylene bridges, each alkylene bridge consisting of one, two, or three carbon atoms, each linking two non-adjacent carbon atoms of the ring system. Cycloalkyl can be fused with aryl or heteroaryl group. In some embodiments, cycloalkyl is benzocondensed. Representative examples of such bridged cycloalkyl ring systems include, but are not limited to, bicyclo[1.1.1]pentane, bicyclo[3.1.1]heptane, bicyclo[2.2.1]heptane, bicyclo[2.2.2]octane, bicyclo[3.2.2]nonane, bicyclo[3.3.1]nonane, bicyclo[4.2.1]nonane, tricyclo[3.3.1.03,7]nonane and tricyclo[3.3.1.13,7]decane (adamantane). The cycloalkyl can be attached to the parent molecular moiety through any substitutable atom contained within the ring system.

The term “cycloalkylene” refers to a divalent cycloalkyl group. An example of a cycloalkylene group includes but not limited to, cyclopropylene, cyclobutylene, cyclopentylene, cyclohexylene, cyclohexenylene, cycloheptylene, cyclooctylene, cyclononylen, cyclodecylen, bicyclo[3.3.0]octylene, bicyclo[3.2.1]octylene, bicyclo[3.3.1]nonylene, and the like.

The term “alkenyl”, employed alone or in combination with other terms, refers to a non-aromatic hydrocarbon radical, straight, branched or cyclic, containing 2-10 carbon atoms and at least one carbon to carbon double bond. In some embodiments, the cyclic refers to monocyclic or multicyclic. In a multicyclic alkenyl, two or more rings can be fused or bridged or spiro together. In some embodiments, one carbon to carbon double bond is present, and up to four non-aromatic carbon-carbon double bonds may be present. Thus, “C₂₋₆ alkenyl” means an alkenyl radical having 2-6 carbon atoms. Alkenyl groups include but are not limited to ethenyl, propenyl, butenyl, 2-methylbutenyl, cyclopentenyl and cyclohexenyl. The straight, branched or cyclic portion of the alkenyl group may contain double bonds and may be substituted if a substituted alkenyl group is indicated.

The term “cycloalkenyl” refers to a non-aromatic hydrocarbon ring system having 3 to 30 carbons (e.g., C₃-C₁₀) and one or more double bonds. Examples include cyclobutenyl, cyclopentenyl, cyclohexenyl, cycloheptenyl etc.

The term “alkynyl”, employed alone or in combination with other terms, refers to a hydrocarbon radical, straight, branched or cyclic, containing 2-10 carbon atoms and at least one carbon to carbon triple bond. In some embodiments, up to three carbon-carbon triple bonds may be present. Thus, “C₂₋₆ alkynyl” means an alkynyl radical having 2-6 carbon atoms. Alkynyl groups include but are not limited to ethynyl, propynyl, butynyl, and 3-methylbutynyl. The straight, branched or cyclic portion of the alkynyl group may contain triple bonds and may be substituted if a substituted alkynyl group is indicated.

The term “halogen” (or “halo”) refers to fluorine, chlorine, bromine and iodine.

The term “alkoxy”, employed alone or in combination with other terms, refers to an alkyl as defined above, which is single bonded to an oxygen atom. The attachment point of an alkoxy radical to a molecule is through the oxygen atom. An alkoxy radical may be depicted as —O-alkyl. The term “C₁₋₁₀ alkoxy” refers to an alkoxy radical containing 1-10 carbon atoms, having straight or branched moieties. Alkoxy group includes but is not limited to, methoxy, ethoxy, propoxy, isopropoxy, butoxy, pentyloxy, hexyloxy, and the like.

The term “cycloalkoxy”, employed alone or in combination with other terms, refers to cycloalkyl as defined above, which is single bonded to an oxygen atom. The attachment point of a cycloalkoxy radical to a molecule is through the oxygen atom. A cycloalkoxy radical may be depicted as —O-cycloalkyl. “C₃₋₁₀ cycloalkoxy” refers to a cycloalkoxy radical containing 3-10 carbon atoms. Cycloalkoxy can be fused with aryl or heteroaryl group. In some embodiments, cycloalkoxy is benzocondensed. Cycloalkoxy group includes but is not limited to, cyclopropoxy, cyclobutoxy, cyclopentyloxy, cyclohexyloxy, and the like.

The term “alkylthio”, employed alone or in combination with other terms, refers to an alkyl radical as defined above, which is single bonded to a sulfur atom. The attachment point of an alkylthio radical to a molecule is through the sulfur atom. An alkylthio radical may be depicted as —S-alkyl. The term “C₁₋₁₀ alkylthio” refers to an alkylthio radical containing 1-10 carbon atoms, having straight or branched moieties. Alkylthio group includes but is not limited to, methylthio, ethylthio, propylthio, isopropylthio, butylthio, hexylthio, and the like.

The term “cycloalkylthio”, employed alone or in combination with other terms, refers to cycloalkyl as defined above, which is single bonded to a sulfur atom. The attachment point of a cycloalkylthio radical to a molecule is through the sulfur atom. A cycloalkylthio radical may be depicted as —S-cycloalkyl. “C₃₋₁₀ cycloalkylthio” refers to a cycloalkylthio radical containing 3-10 carbon atoms. Cycloalkylthio can be fused with aryl or heteroaryl group. In some embodiments, cycloalkylthio is benzocondensed. Cycloalkylthio group includes but is not limited to, cyclopropylthio, cyclobutylthio, cyclohexylthio, and the like.

The term “alkylamino”, employed alone or in combination with other terms, refers to an alkyl as defined above, which is single bonded to a nitrogen atom. The attachment point of an alkylamino radical to a molecule is through the nitrogen atom. An alkylamino radical may be depicted as —NH (alkyl). The term “C₁₋₁₀ alkylamino” refers to an alkylamino radical containing 1-10 carbon atoms, having straight or branched moieties. Alkylamino group includes but is not limited to, methylamino, ethylamino, propylamino, isopropylamino, butylamino, hexylamoino, and the like.

The term “cycloalkylamino”, employed alone or in combination with other terms, refers to cycloalkyl as defined above, which is single bonded to a nitrogen atom. The attachment point of a cycloalkylamino radical to a molecule is through the nitrogen atom. A cycloalkylamino radical may be depicted as —NH (cycloalkyl). “C₃₋₁₀ cycloalkylamino” refers to a cycloalkylamino radical containing 3-10 carbon atoms. Cycloalkylamino can be fused with aryl or heteroaryl group. In some embodiments, cycloalkylamino is benzocondensed. Cycloalkylamino group includes but is not limited to, cyclopropylamino, cyclobutylamino, cyclohexylamino, and the like.

The term “di(alkyl)amino”, employed alone or in combination with other terms, refers to two alkyl as defined above, which are single bonded to a nitrogen atom. The attachment point of an di(alkyl)amino radical to a molecule is through the nitrogen atom. A di(alkyl)amino radical may be depicted as —N(alkyl) 2. The term “di(C₁₋₁₀ alkyl)amino” refers to a di(C₁₋₁₀ alkyl)amino radical wherein the alkyl radicals each independently contains 1-10 carbon atoms, having straight or branched moieties.

The term “aryl”, employed alone or in combination with other terms, refers to a monovalent, monocyclic-, bicyclic- or tricyclic aromatic hydrocarbon ring system having 6, 7, 8, 9, 10, 11, 12, 13 or 14 carbon atoms (a “C₆₋₁₄ aryl” group), particularly a ring having 6 carbon atoms (a “C₆ aryl” group), e.g. a phenyl group; or a ring having 10 carbon atoms (a “C₁₀ aryl” group), e.g. a naphthyl group; or a ring having 14 carbon atoms, (a “C₁₄ aryl” group), e.g. an anthranyl group. Aryl can be fused with cycloalkyl or heterocycle group.

Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by removing “-yl” and adding “-idene” to the name of the corresponding univalent radical, e.g., a naphthyl group with two points of attachment is termed naphthylidene.

The term “heteroaryl”, employed alone or in combination with other terms, refers to a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom selected from N, O and S. Heteroaryl can be fused with cycloalkyl or heterocycle group. In some embodiments, “heteroaryl” refers to a 5- to 8-membered monocyclic aromatic ring containing one or more, for example, from 1 to 4, or, in some embodiments, from 1 to 3, heteroatoms selected from N, O and S, with the remaining ring atoms being carbon; or a 8- to 12-membered bicyclic aromatic ring system containing one or more, for example, from 1 to 6, or, in some embodiments, from 1 to 4, or, in some embodiments, from 1 to 3, heteroatoms selected from N, O and S, with the remaining ring atoms being carbon; or a 11- to 14-membered tricyclic aromatic ring system containing one or more, for example, from 1 to 8, or, in some embodiments, from 1 to 6, or, in some embodiments, from 1 to 4, or in some embodiments, from 1 to 3, heteroatoms selected from N, O and S, with the remaining ring atoms being carbon.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of heteroaryl groups include, but are not limited to, pyrid-2-yl, pyrid-3-yl, pyrid-4-yl, pyrazin-2-yl, pyrazin-3-yl, pyrimidin-2-yl, pyrimidin-4-yl, pyrimidin-5-yl, pyrimidin-6-yl, pyrazol-1-yl, pyrazol-3-yl, pyrazol-4-yl, pyrazol-5-yl, imidazol-1-yl, imidazol-2-yl, imidazol-4-yl, imidazol-5-yl, pyridazinyl, triazinyl, pyrrolyl, oxazolyl, isoxazolyl, thiazolyl, isothiazolyl, thiadiazolyl, triazolyl, tetrazolyl, thienyl, furyl.

Further heteroaryl groups include but are not limited to indolyl, benzothienyl, benzofuryl, benzoimidazolyl, benzotriazolyl, quinoxalinyl, quinolinyl, and isoquinolinyl. “Heteroaryl” is also understood to include the N-oxide derivative of any nitrogen-containing heteroaryl.

Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene.

The term “heterocycle”, employed alone or in combination with other terms, (and variations thereof such as “heterocyclic”, or “heterocyclyl”) broadly refers to a saturated or unsaturated mono- or multicyclic (e.g. bicyclic or tricyclic) aliphatic ring system, usually with 3 to 16 ring atoms, wherein at least one (e.g. 2, 3 or 4) ring atom is heteroatom independently selected from O, S, N and P (preferably O, S, N). In a multicyclic heterocycle, two or more rings can be fused or bridged or spiro together. Heterocycle can be fused with aryl or heteroaryl group. In some embodiments, heterocycle is benzocondensed. Heterocycle also includes ring systems substituted with one or more oxo or imino moieties. In some embodiments, the C, N, S and P atoms in the heterocycle ring are optionally substituted by oxo. In some embodiments, the C, S and P atoms in the heterocycle ring are optionally substituted by imino, and imino can be unsubstituted or substituted. The point of the attachment may be carbon atom or heteroatom in the heterocyclic ring, provided that attachment results in the creation of a stable structure. When the heterocyclic ring has substituents, it is understood that the substituents may be attached to any atom in the ring, whether a heteroatom or a carbon atom, provided that a stable chemical structure result.

Suitable heterocycles include, for example, pyrrolidin-1-yl, pyrrolidin-2-yl, pyrrolidin-3-yl, imidazolidin-1-yl, imidazolidin-2-yl, imidazolidin-3-yl, imidazolidin-4-yl, imidazolidin-5-yl, pyrazolidin-1-yl, pyrazolidin-2-yl, pyrazolidin-3-yl, pyrazolidin-4-yl, pyrazolidin-5-yl, piperidin-1-yl, piperidin-2-yl, piperidin-3-yl, piperidin-4-yl, piperazin-1-yl, piperazin-2-yl, piperazin-3-yl, hexahydropyridazin-1-yl, hexahydropyridazin-3-yl, hexahydropyridazin-4-yl and tetrahydropyridyl. Morpholinyl groups are also contemplated, such as morpholin-1-yl, morpholin-2-yl, morpholin-3-yl and morpholin-4-yl. Examples of heterocycle with one or more oxo moieties include but are not limited to, piperidinyl N-oxide, morpholinyl-N-oxide, 1-oxo-thiomorpholinyl and 1,1-dioxo-thiomorpholinyl. Bicyclic heterocycles include, for example:

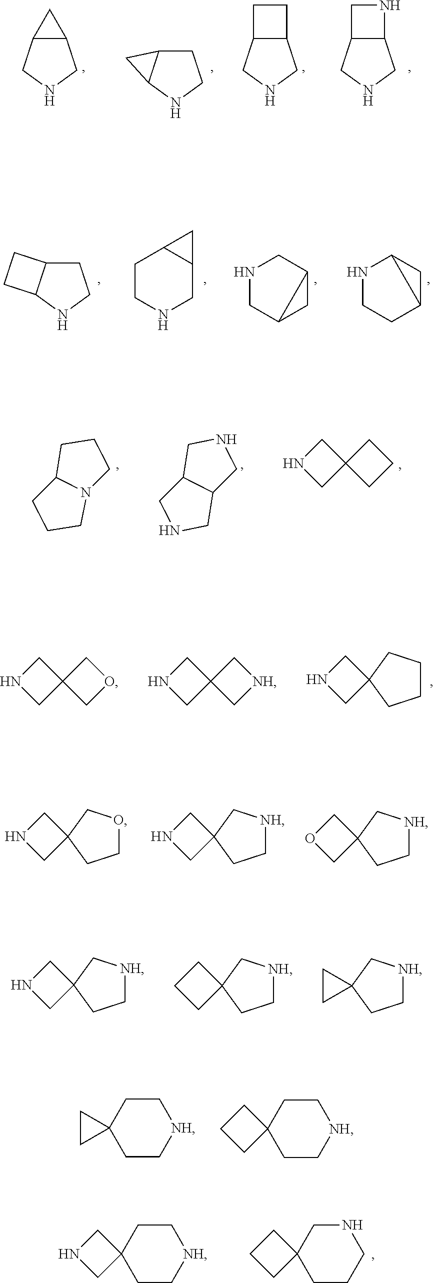

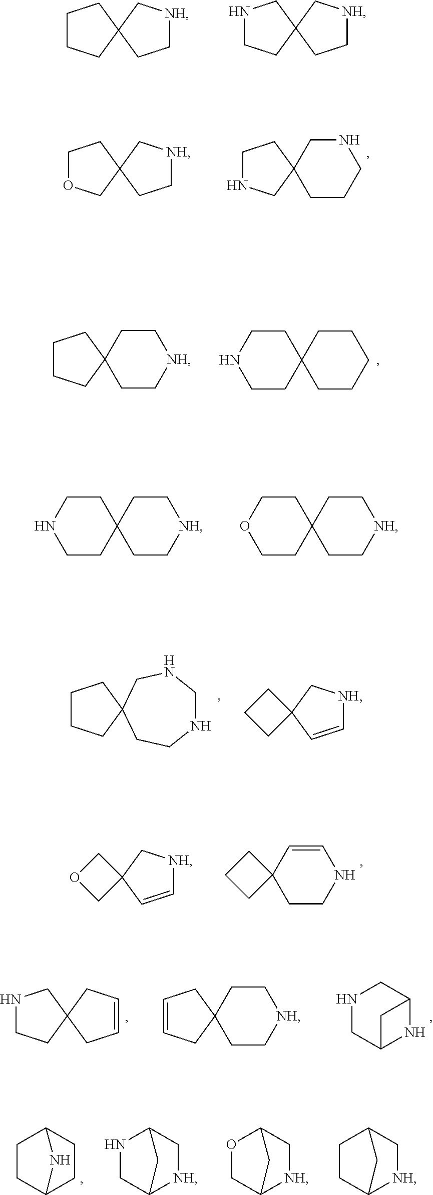

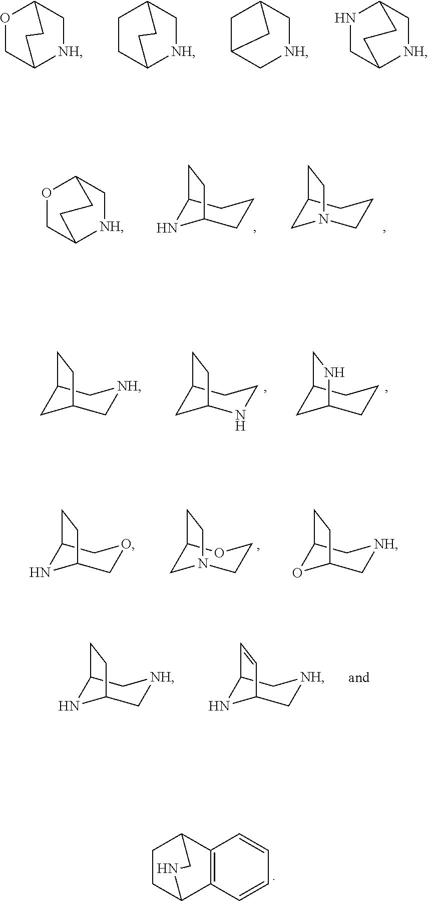

The term “heterocyclylene” refers to a divalent heterocyclyl group. An example of a heterocyclylene group is divalent piperidine. The heterocyclylene is attached to the rest of the molecule through a single bond and to the radical group through a single bond. The points of attachment of the heterocyclylene to the rest of the molecule and to the radical group can be through one atom in the heterocyclylene or through two separate atoms of the heterocyclylene. In addition, the point of attachment to the rest of the molecule and/or to the radical group may be through a carbon atom or a heteroatom.

As used herein, “aryl-alkyl” refers to an alkyl moiety as defined above substituted by an aryl group as defined above. Exemplary aryl-alkyl groups include but are not limited to benzyl, phenethyl and naphthylmethyl groups. In some embodiments, aryl-alkyl groups have 7-20 or 7-11 carbon atoms. When used in the phrase “aryl-C₁₋₄ alkyl”, the term “C₁₋₄” refers to the alkyl portion of the moiety and does not describe the number of atoms in the aryl portion of the moiety.

As used herein, “heterocyclyl-alkyl” refers to alkyl as defined above substituted by heterocyclyl as defined above. When used in the phrase “heterocyclyl-C₁₋₄ alkyl”, the term “C₁₋₄” refers to the alkyl portion of the moiety and does not describe the number of atoms in the heterocyclyl portion of the moiety.

As used herein, “cycloalkyl-alkyl” refers to alkyl as defined above substituted by cycloalkyl as defined above. When used in the phrase “C₃₋₁₀ cycloalkyl-C₁₋₄ alkyl”, the term “C₃₋₁₀” refers to the cycloalkyl portion of the moiety and does not describe the number of atoms in the alkyl portion of the moiety, and the term “C₁₋₄” refers to the alkyl portion of the moiety and does not describe the number of atoms in the cycloalkyl portion of the moiety.

As used herein, “heteroaryl-alkyl” refers to alkyl as defined above substituted by heteroaryl as defined above. When used in the phrase “heteroaryl-C₁₋₄ alkyl”, the term “C₁₋₄” refers to the alkyl portion of the moiety and does not describe the number of atoms in the heteroaryl portion of the moiety.

For avoidance of doubt, reference, for example, to substitution of alkyl, cycloalkyl, heterocyclyl, aryl and/or heteroaryl refers to substitution of each of those groups individually as well as to substitutions of combinations of those groups. That is, if R is aryl-C₁₋₄ alkyl and may be unsubstituted or substituted with at least one substituent, such as one, two, three, or four substituents, independently selected from R^{X}, it should be understood that the aryl portion may be unsubstituted or substituted with at least one substituent, such as one, two, three, or four substituents, independently selected from RX and the alkyl portion may also be unsubstituted or substituted with at least one substituent, such as one, two, three, or four substituens, independently selected from RX.

The term “pharmaceutically acceptable salts” refers to salts prepared from pharmaceutically acceptable non-toxic bases or acids including inorganic or organic bases and inorganic or organic acids. Salts derived from inorganic bases may be selected, for example, from aluminum, ammonium, calcium, copper, ferric, ferrous, lithium, magnesium, manganic, manganous, potassium, sodium and zinc salts. Further, for example, the pharmaceutically acceptable salts derived from inorganic bases may be selected from ammonium, calcium, magnesium, potassium and sodium salts. Salts in the solid form may exist in one or more crystalline forms, or polymorphs, and may also be in the form of solvates, such as hydrates. Salts derived from pharmaceutically acceptable organic non-toxic bases may be selected, for example, from salts of primary, secondary and tertiary amines, substituted amines including naturally occurring substituted amines, cyclic amines and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine and tripropylamine, tromethamine.

When the compound disclosed herein is basic, salts may be prepared using at least one pharmaceutically acceptable non-toxic acid, selected from inorganic and organic acids. Such acid may be selected, for example, from acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric and p-toluenesulfonic acids. In some embodiments, such acid may be selected, for example, from citric, hydrobromic, hydrochloric, maleic, phosphoric, sulfuric, fumaric and tartaric acids.

The terms “administration of” and or “administering” a compound or a pharmaceutically acceptable salt should be understood to mean providing a compound or a pharmaceutically acceptable salt thereof to the individual in recognized need of treatment.

The term “effective amount” means the amount of the a compound or a pharmaceutically acceptable salt that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to a pharmaceutical composition is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients.

The term “pharmaceutically acceptable” it is meant compatible with the other ingredients of the formulation and not unacceptably deleterious to the recipient thereof.

The term “subject” as used herein in reference to individuals suffering from a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “protecting group” or “Pg” refers to a substituent that can be commonly employed to block or protect a certain functionality while reacting other functional groups on the compound. For example, an “amino-protecting group” is a substituent attached to an amino group that blocks or protects the amino functionality in the compound. Suitable amino-protecting groups include but are not limited to acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include but are not limited to acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl) ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The term “NH protecting group” as used herein includes, but not limited to, trichloroethoxycarbonyl, tribromoethoxycarbonyl, benzyloxycarbonyl, para-nitrobenzylcarbonyl, ortho-bromobenzyloxycarbonyl, chloroacetyl, dichloroacetyl, trichloroacetyl, trifluoroacetyl, phenylacetyl, formyl, acetyl, benzoyl, tert-amyloxycarbonyl, tert-butoxycarbonyl, para-methoxybenzyloxycarbonyl, 3,4-dimethoxybenzyl-oxycarbonyl, 4-(phenylazo)-benzyloxycarbonyl, 2-furfuryloxycarbonyl, diphenylmethoxycarbonyl, 1,1-dimethylpropoxy-carbonyl, isopropoxycarbonyl, phthaloyl, succinyl, alanyl, leucyl, 1-adamantyloxycarbonyl, 8-quinolyloxycarbonyl, benzyl, diphenylmethyl, triphenylmethyl, 2-nitrophenylthio, methanesulfonyl, para-toluenesulfonyl, N,N-dimethylaminomethylene, benzylidene, 2-hydroxybenzylidene, 2-hydroxy-5-chlorobenzylidene, 2-hydroxy-1-naphthylmethylene, 3-hydroxy-4-pyridylmethylene, cyclohexylidene, 2-ethoxycarbonylcyclohexylidene, 2-ethoxycarbonylcyclopentylidene, 2-acetylcyclohexylidene, 3,3-dimethyl-5-oxycyclo-hexylidene, diphenylphosphoryl, dibenzylphosphoryl, 5-methyl-2-oxo-2H-1,3-dioxol-4-yl-methyl, trimethylsilyl, triethylsilyl and triphenylsilyl.

The term “C(O)OH protecting group” as used herein includes, but not limited to, methyl, ethyl, n-propyl, isopropyl, 1,1-dimethylpropyl, n-butyl, tert-butyl, phenyl, naphthyl, benzyl, diphenylmethyl, triphenylmethyl, para-nitrobenzyl, para-methoxy benzyl, bis(para-methoxyphenyl)methyl, acetylmethyl, benzoylmethyl, para-nitrobenzoylmethyl, para-bromobenzoylmethyl, para-methanesulfonylbenzoylmethyl, 2-tetrahydropyranyl, 2-tetrahydrofuranyl, 2,2,2-trichloro-ethyl, 2-(trimethylsilyl)ethyl, acetoxymethyl, propionyloxymethyl, pivaloyloxymethyl, phthalimidomethyl, succinimidomethyl, cyclopropyl, cyclobutyl, cyclopentyl, cyclohexyl, methoxymethyl, methoxyethoxymethyl, 2-(trimethylsilyl) ethoxymethyl, benzyloxymethyl, methylthiomethyl, 2-methylthioethyl, phenylthiomethyl, 1,1-dimethyl-2-propenyl, 3-methyl-3-butenyl, allyl, trimethylsilyl, triethylsilyl, triisopropylsilyl, diethylisopropylsilyl, tert-butyldimethylsilyl, tert-butyldiphenylsilyl, diphenylmethylsilyl and tert-butylmethoxyphenylsilyl.

The term “OH or SH protecting group” as used herein includes, but not limited to, benzyloxycarbonyl, 4-nitrobenzyloxycarbonyl, 4-bromobenzyloxycarbonyl, 4-methoxybenzyloxycarbonyl, 3,4-dimethoxybenzyloxycarbonyl, methoxycarbonyl, ethoxycarbonyl, tert-butoxycarbonyl, 1,1-dimethylpropoxycarbonyl, isopropoxycarbonyl, isobutyloxycarbonyl, diphenylmethoxycarbonyl, 2,2,2-trichloroethoxycarbonyl, 2,2,2-tribromoethoxycarbonyl, 2-(trimethylsilyl) ethoxycarbonyl, 2-(phenylsulfonyl) ethoxycarbonyl, 2-(triphenylphosphonio) ethoxycarbonyl, 2-furfuryloxycarbonyl, 1-adamantyloxycarbonyl, vinyloxycarbonyl, allyloxycarbonyl, 4-ethoxy-1-naphthyloxycarbonyl, 8-quinolyloxycarbonyl, acetyl, formyl, chloroacetyl, dichloroacetyl, trichloroacetyl, trifluoroacetyl, methoxyacetyl, phenoxyacetyl, pivaloyl, benzoyl, methyl, tert-butyl, 2,2,2-trichloroethyl, 2-trimethylsilylethyl, 1,1-dimethyl-2-propenyl, 3-methyl-3-butenyl, allyl, benzyl (phenylmethyl), para-methoxybenzyl, 3,4-dimethoxybenzyl, diphenylmethyl, triphenylmethyl, tetrahydrofuryl, tetrahydropyranyl, tetrahydrothiopyranyl, methoxymethyl, methylthiomethyl, benzyloxymethyl, 2-methoxyethoxymethyl, 2,2,2-trichloro-ethoxymethyl, 2-(trimethylsilyl) ethoxymethyl, 1-ethoxyethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl and tert-butylmethoxyphenylsilyl.

Geometric isomers may exist in the present compounds. Compounds of this invention may contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers. Substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantane ring system are designated as being of Z or E relative configuration. For examples, see C. D. Jones, M. Kaselj, R. N. Salvatore, W. J. le Noble J. Org. Chem. 1998, 63, 2758-2760.

Compounds of this invention may contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration present in the higher amount, preferably an excess of about 85-90%, more preferably an excess of about 95-99%, and still more preferably an excess greater than about 99%. Accordingly, this invention includes racemic mixtures, relative and absolute stereoisomers, and mixtures of relative and absolute stereoisomers. When the carbon marked with * is asymmetric center. In an embodiment, the asymmetric center is S configured carbon atom, enriched S configured carbon atom or racemic carbon atom. In an embodiment, the asymmetric center is R configured carbon atom, enriched R configured carbon atom or racemic carbon atom. Isotope Enriched or Labeled Compounds.

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, nitrogen, oxygen, phosphorous, sulfur, fluorine, chlorine and iodine include, but are not limited to, ²H, ³H, ¹³C, ¹⁴C, ¹⁵N, ¹⁸O, ³²P, ³⁵S, ¹⁸F, ³⁶Cl and ¹²⁵I. Compounds that contain other isotopes of these and/or other atoms are within the scope of this invention.

In another embodiment, the isotope-labeled compounds contain deuterium (²H), tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuterated acid such as D₂SO₄/D₂O.

The isotope-labeled compounds of the invention may be used as standards to determine the effectiveness of GLP-1R inhibitors in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al, J. Labelled Compounds. Radiopharmaceuticals, 36 (10), 927-932 (1995); Kushner et al., Can. J. Physiol. Pharmacology, 77, 79-88 (1999).

In addition, non-radioactive isotope containing drugs, such as deuterated drugs called “heavy drugs” can be used for the treatment of diseases and conditions related to GLP-1R activity. Increasing the amount of an isotope present in a compound above its natural abundance is called enrichment. Examples of the amount of enrichment include but are not limited to from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

In an Embodiment (1), this invention provides to a compound of formula (I),

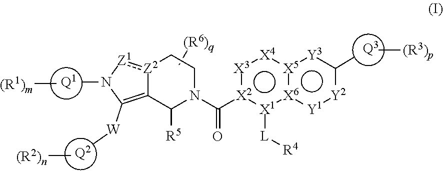

or a pharmaceutically acceptable salt thereof, wherein:
[symbol] is a double bond or a single bond;
X¹, X², X⁵ and X⁶ are independently selected from N and C;
X³ is selected from O, S, N, NR^{7.3} and CR^{7.3};
X⁴ is absent or selected from N, C(O) and CR^{7.4};
Y¹ is selected from N and CR^{8.1};
Y² is selected from N and CR^{8.2};
Y³ is selected from N and CR^{8.3};
Z¹ is selected from N and C(O);
Z² is selected from N and C;
W is selected from

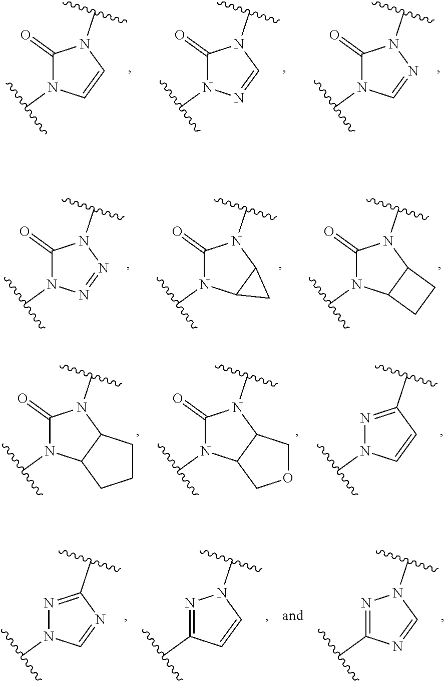

which are each unsubstituted or substituted with at least one substituent, independently selected from RX;
L is selected from —CR^{C0}R^{D0} (CR^{C0}R^{D0})ᵤ—, (CR^{C0}R^{D0})ᵤNR^{A0}(CR^{C0}R^{D0})ₜ— and —(CR^{C0}R^{D0})ᵤS(O), (CR^{C0}R^{D0})ₜ—;
Q¹, Q² and Q³ is selected from C₃₋₁₀ cycloalkyl, heterocyclyl, aryl and heteroaryl;
each R¹, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═NR^{E1})R^{A1}, —C(═N—OR^{B1})R^{A1}, —C(O)OR^{A1}, —OC(O)R^{A1}, —C(O)NR^{A1}R^{B1}, —C(O)NR^{A1}S(O)ᵣR^{A1}, —C(O)NR^{A1}S(O)₂OR^{A1}, —C(O)NR^{A1}S(O)ᵣNR^{A1}R^{B1}, —C(O)NR^{A1}S(O)(═NR^{E1})R^{B1}, —C(O)NR^{A1}S(O)(═NR^{E1}) NR^{A1}R^{B1}, —NR^{A1}C(O)R^{B1}, —C(═NR^{E1}) NR^{A1}R^{B1}, —NR^{A1}C(═NR^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1}) NR^{A1}R^{B1}, —S(O)ᵣR^{A1}, —S(O)(═NR^{E1})R^{B1}, —N═S(O)R^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}, —S(O)ᵣNR^{A1}R^{B1}, —S(O)(═NR^{E1}) NR^{A1}R^{B1}, —NR^{A1}S(O)₂NR^{A1}R^{B1}, —NR^{A1}S(O)(═NR^{E1}) NR^{A1}R^{B1}, —P(O)R^{A1}R^{B1} and —P(O)(OR^{A1})(OR^{B1}), wherein alkyl, alkenyl, alkynyl, cycloalkyl, heterocyclyl, aryl and heteroaryl are each unsubstituted or substituted with at least one substituent, independently selected from R^{X1};
each R², at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —C(O)NR^{A2}S(O)(═NR^{E2})R^{B2}, —C(O)NR^{A2}S(O)(═NR^{E2}) NR^{A2}R^{B2}, —NR^{A2}C(O)R^{B2}, —C(═NR^{E2}) NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2})NR^{A2}R^{B2}, —S(O)(═NR^{E2})R^{B2}, —N═S(O)R^{A2}R^{B2}, —NR^{A2}S(O)(═NR^{E2})R^{B2}, —S(O)(═NR^{E2}) NR^{A2}R^{B2} and —NR^{A2}S(O)(═NR^{E2}) NR^{A2}R^{B2}, wherein alkyl, alkenyl, alkynyl, cycloalkyl, heterocyclyl, aryl and heteroaryl are each unsubstituted or substituted with at least one substituent, independently selected from R^{X2};
each R³, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —C(O)NR^{A3}S(O)ᵣR^{A3}, —C(O)NR^{A3}S(O)₂OR^{A3}, —C(O)NR^{A3}S(O)ᵣNR^{A3}R^{B3}, —C(O)NR^{A3}S(O)(═NR^{E3})R^{B3}, —C(O)NR^{A3}S(O)(═NR^{E3}) NR^{A3}R^{B3}, —NR^{A3}C(O)R^{B3}, —C(═NR^{E3}) NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3}) NR^{A3}R^{B3}, —S(O)ᵣR^{A3}, —S(O)(═NR^{E3})R^{B3}, —N═S(O)R^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3}) NR^{A3}R^{B3}, —NR^{A3}S(O)₂NR^{A3}R^{B3}, —NR^{A3}S(O)(═NR^{E3}) NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R^{X3};
R⁴ is selected from —C(O)OH, —C(O)NHS(O)ᵣR^{A4}, —(CR^{c1}R^{d1})ₜNHS(O)ᵣR^{A4}, heterocyclyl and heteroaryl;
R⁵ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A5}R^{B5}, —OR^{A5} and —C(O)R^{A5}, wherein alkyl, alkenyl, alkynyl, cycloalkyl, and heterocyclyl are each unsubstituted or substituted with at least one substituent, independently selected from R^{X5};
each R⁶, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A6}R^{B6}, —OR^{A6} and —C(O)R^{A6}, wherein alkyl, alkenyl, alkynyl, cycloalkyl, and heterocyclyl are each unsubstituted or substituted with at least one substituent, independently selected from R^{X6};
or “R⁵ and R⁶” or “two of R⁶” together with the atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X6.}
R^{7.3} and R^{7.4} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A7}R^{B7}, —OR^{A7}, —C(O)R^{A7}, —C(═NR^{E7})R^{A7}, —C(═N—OR^{B7})R^{A7}, —C(O)OR^{A7}, —OC(O)R^{A7}, —C(O)NR^{A7}R^{B7}, —C(O)NR^{A7}S(O)ᵣR^{A7}, —C(O)NR^{A7}S(O)₂OR^{A7}, —C(O)NR^{A7}S(O)ᵣNR^{A7}R^{B7}, —C(O)NR^{A7}S(O)(═NR^{E7})R^{B7}, —C(O)NR^{A7}S(O)(═NR^{E7}) NR^{A7}R^{B7}, —NR^{A7}C(O)R^{B7}, —C(═NR^{E7}) NR^{A7}R^{B7}, —NR^{A7}C(═NR^{E7})R^{B7}, —OC(O)NR^{A7}R^{B7}, —NR^{A7}C(O)OR^{B7}, —NR^{A7}C(O)NR^{A7}R^{B7}, —NR^{A7}C(S)NR^{A7}R^{B7}, —NR^{A7}C(═NR^{E7}) NR^{A7}R^{B7}, —S(O)ᵣR^{A7}, —S(O)(═NR^{E7})R^{B7}, —N═S(O)R^{A7}R^{B7}, —S(O)₂OR^{A7}, —OS(O)₂R^{A7}, —NR^{A7}S(O)ᵣR^{B7}, —NR^{A7}S(O)(═NR^{E7})R^{B7}, —S(O)ᵣNR^{A7}R^{B7}, —S(O)(═NR^{E7}) NR^{A7}R^{B7}, —NR^{A7}S(O)₂NR^{A7}R^{B7}, —NR^{A7}S(O)(═NR^{E7}) NR^{A7}R^{B7}, —P(O)R^{A7}R^{B7} and —P(O)(OR^{A7})(OR^{B7}), wherein alkyl, alkenyl, alkynyl, cycloalkyl, heterocyclyl, aryl and heteroaryl are each unsubstituted or substituted with at least one substituent, independently selected from R^{X7};
R^{8.1}. R^{8.2} and R^{8.3} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A8}R^{B8}, —OR^{A8}, —C(O)R^{A8}, —C(═NR^{E8})R^{A8}, —C(═N—OR^{B8})R^{A8}, —C(O)OR^{A8}, —OC(O)R^{A8}, —C(O)NR^{A8}R^{B8}, —C(O)NR^{A8}S(O)ᵣR^{A8}, —C(O)NR^{A8}S(O)₂OR^{A8}, —C(O)NR^{A8}S(O)ᵣNR^{A8}R^{B8}, —C(O)NR^{A8}S(O)(═NR^{E8})R^{B8}, —C(O)NR^{A8}S(O)(═NR^{E8}) NR^{A8}R^{B8}, —NR^{A8}C(O)R^{B8}, —C(═NR^{E8}) NR^{A8}R^{B8}, —NR^{A8}C(═NR^{E8})R^{B8}, —OC(O)NR^{A8}R^{B8}, —NR^{A8}C(O)OR^{B8}, —NR^{A8}C(O)NR^{A8}R^{B8}, —NR^{A8}C(S)NR^{A8}R^{B8}, —NR^{A8}C(═NR^{E8}) NR^{A8}R^{B8}, —S(O)ᵣR^{A8}, —S(O)(═NR^{E8})R^{B8}, —N═S(O)R^{A8}R^{B8}, —S(O)₂OR^{A8}, —OS(O)₂R^{A8}, —NR^{A8}S(O)ᵣR^{B8}, —NR^{A8}S(O)(═NR^{E8})R^{B8}, —S(O)ᵣNR^{A8}R^{B8}, —S(O)(═NR^{E8})NR^{A8}R^{B8}, —NR^{A8}S(O)₂NR^{A8}R^{B8}, —NR^{A8}S(O)(═NR^{E8}) NR^{A8}R^{B8}, —P(O)R^{A8}R^{B8} and —P(O)(OR^{A8})(OR^{B8}), wherein alkyl, alkenyl, alkynyl, cycloalkyl, heterocyclyl, aryl and heteroaryl are each unsubstituted or substituted with at least one substituent, independently selected from R^{X8};
R^{A0} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
each R^{A1} and R^{B1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X1.}
or each “R^{A1} and R^{B1}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X1} groups;
each R^{A2} and R^{B2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X2};
or each “R^{A2} and R^{B2}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X2} groups;
each R^{A3} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X3.}
or each “R^{A3} and R^{B3}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X3} groups;
R^{A4} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X4.}
each R^{A5} and R^{B5} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X5};
or each “R^{A5} and R^{B5}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X5} groups;
each R^{A6} and R^{B6} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X6};
or each “R^{A6} and R^{B6}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X6} groups;
each R^{A7} and R^{B7} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X7},
or each “R^{A7} and R^{B7}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X7} groups;
each R^{A8} and R^{B8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl, and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X8.}
or each “R^{A8} and R^{B8}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X8} groups;
each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
or R^{C0} and R^{D0} together with the carbon atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X0};
each R^{E1}, R^{E2}, R^{E3}, R^{E7} and R^{E8} are independently selected from hydrogen, C₁₋₁₀ alkyl, CN, NO₂, —OR^{a1}, —SR^{a1}, —S(O)ᵣRal, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from RX;
provided that when Z¹ is N, X² is C, X³ is CR^{7.3} or N, X⁴ is absent, R⁵ and R⁶ or any two of R⁶ do not form any ring, W is

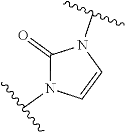 [SMILES: *n1ccn(*)c1=O]

and L is selected from —CR^{C0}R^{D0}(CR^{C0}R^{D0})ᵤ—, then at least one of R^{C0} and R^{D0} is substituted with R^{X0}, and R^{X0} is selected from CN, C₂₋₁₀ alkenyl and C₂₋₁₀ alkynyl;
each R^{X}, R^{X0}, R^{X1}, R^{X2}, R^{X3}, R^{X4}, R^{X5}, R^{X6}, R^{X7} and R^{X8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1},
(CR^{c1}R^{d1})ₜC(═N—OR^{b1})R^{a1}, —(CR^{c1}R^{d1})ₜC(O)OR^{b1}, —(CR^{c1}R^{d1})ₜC(O)R^{b1}, —(CR^{c1}R^{d1})ₜC(O)NR^{a1}R^{b1}, —(CR^{c1}R^{d1})ₜNR^{a1}C(O)R^{b1}, —(CR^{c1}R^{d1})ₜC(═NR^{e1}) NR^{a1}R^{b1}, —(CR^{c1}R^{d1})ₜNR^{a1}C(═NR^{e1})R^{b1}, —(CR^{c1}R^{d1})ₜC(O)NR^{a1}R^{b1}, —(CR^{c1}R^{d1})ₜNR^{a1}C(O)OR^{b1}, —(CR^{c1}R^{d1})ₜNR^{a1}C(O)NR^{a1}R^{b1}, —(CR^{c1}R^{d1})ₜNR^{a1}C(S)NR^{a1}R^{b1}, —(CR^{c1}R^{d1})ₜNR^{a1}C(═NR^{e1}) NR^{a1}R^{b1}, —(CR^{c1}R^{d1})ₜS(O)ᵣR^{b1}, —(CR^{c1}R^{d1})S(O)(═NR^{e1})R^{b1}, —(CR^{c1}R^{d1})ₜN═S(O)R^{a1}R^{b1}, —(CR^{c1}R^{d1})ₜS(O)₂OR^{b1}, —(CR^{c1}R^{d1}) OS(O)₂R^{b1}, —(CR^{c1}R^{d1})ₜNR^{a1}S(O)ₜR^{b1}, —(CR^{c1}R^{d1})ₜNR^{a1}S(O)(═NR^{e1})R^{b1}, —(CR^{c1}R^{d1})ₜS(O)ᵣNR^{a1}R^{b1}, —(CR^{c1}R^{d1})ₜS(O)(═NR^{e1}) NR^{a1}R^{b1}, —(CR^{c1}R^{d1})ₜNR^{a1}S(O)₂NR^{a1}R^{b1}, wherein alkyl, alkenyl, alkynyl, cycloalkyl, heterocyclyl, aryl and heteroaryl are each unsubstituted or substituted with at least one substituent, independently selected from RY;
each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from RY;
or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 RY groups;
each R^{e1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from RY;
or R^{e1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 RY groups;
each R^{e1} is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR^{a2}R^{b2} and —C(O)NR^{a2}R^{b2};
each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})ₜNR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a2}, —(CR^{c2}R^{d2})ₜC(═N—OR^{b2})R^{a2}, —(CR^{c2}R^{d2})ₜC(O)OR^{b2}, (CR^{c2}R^{d2})ₜOC(O)R^{b2}, —(CR^{c2}R^{d2})ₜC(O)NR^{a2}R^{b2}, —(CR^{c2}R^{d2})ₜNR^{a2}C(O)R^{b2}, —(CR^{c2}R^{d2})ₜC(═NR^{e2})NR^{a2}R^{b2}, —(CR^{c2}R^{d2}), OC(O)NR^{a2}R^{b2}, —(CR^{c2}R^{d2})ₜNR^{a2}C(O)OR^{b2}, —(CR^{c2}R^{d2})ₜNR^{a2}C(O)NR^{a2}R^{b2}, —(CR^{c2}R^{d2})ₜNR^{a2}C(S)NR^{a2}R^{b2}, —(CR^{c2}R^{d2})ₜNR^{a2}C(═NR^{e2})NR^{a2}R^{b2}, —(CR^{c2}R^{d2})ₜS(O)ᵣR^{b2}, —(CR^{c2}R^{d2})ₜS(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2}), OS(O)₂R^{b2}, —(CR^{c2}R^{d2})ₜNR^{a2}S(O)ᵣR^{b2}, —(CR^{c2}R^{d2})ₜNR^{a2}S(O)(═NR^{e2})R^{b2}, —(CR^{c2}R^{d2})ₜS(O)ᵣNR^{a2}R^{b2}, —(CR^{c2}R^{d2})ₜS(O)(═NR^{e2})NR^{a2}R^{b2}, —(CR^{c2}R^{d2})ₜNR^{a2}S(O)₂NR^{a2}R^{b2}, —(CR^{c2}R^{d2})ₜNR^{a2}S(O)(═NR^{e2})NR^{a2}R^{b2}, —(CR^{c2}R^{d2})ₜP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;
each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{a2} and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{c2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{c2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O) C₁₋₄ alkyl, —C(O) C₃₋₁₀ cycloalkyl, —C(O)OC₁₋₄ alkyl, —C(O)OC₃₋₁₀ cycloalkyl, —C(O)N(C₁₋₄ alkyl) 2, —C(O)N(C₃₋₁₀ cycloalkyl) 2, —S(O)₂C₁₋₄ alkyl, —S(O)₂C₃₋₁₀ cycloalkyl, —S(O)₂N(C₁₋₄ alkyl) 2 and —S(O)₂N(C₃₋₁₀ cycloalkyl) 2; m, n, p and q are independently selected from 0, 1, 2, 3 and 4;
each r is independently selected from 0, 1 and 2;
each t is independently selected from 0, 1, 2, 3 and 4;
each u is independently selected from 0, 1, 2, 3 and 4.

In another Embodiment, the invention provides a compound of Embodiment (1) or a pharmaceutically acceptable salt thereof, wherein,
X¹, X², X⁵ and X⁶ are independently selected from N and C;
X³ is selected from O, S, N, NR^{7.3} and CR^{7.3};
X⁴ is absent or selected from N, C(O) and CR^{7.4};
Y¹ is selected from N and CR^{8.1};
Y² is selected from N and CR^{8.2;}
Y³ is selected from N and CR^{8.3;}
Z¹ is selected from N and C(O);
Z² is selected from N and C;
W is selected from

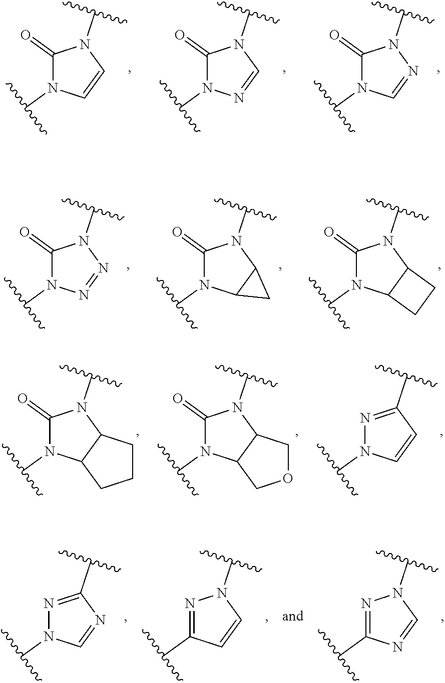

which are each unsubstituted or substituted with at least one substituent, independently selected from RX;
L is selected from —CR^{C0}R^{D0} (CR^{C0}R^{D0})ᵤ—, —(CR^{C0}R^{D0})ᵤO(CR^{C0}R^{D0})ₜ—, (CR^{C0}R^{D0})ᵤNR^{A0}(CR^{C0}R^{D0})ₜ— and —(CR^{C0}R^{D0}) US(O), (CR^{C0}R^{D0})ₜ—;
Q¹, Q² and Q³ is selected from C₃₋₁₀ cycloalkyl, heterocyclyl, aryl and heteroaryl;
provided that when Z¹ is N, X² is C, X⁴ is absent, R⁵ and R⁶ or any two of R⁶ do not form any ring, W is

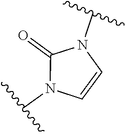 [SMILES: *n1ccn(*)c1=O]

and L is selected from —CR^{C0}R^{D0}(CR^{C0}R^{D0})ᵤ—, then at least one of R^{C0} and R^{D0} is substituted with R^{X0}, and R^{X0} is selected from CN, C₂₋₁₀ alkenyl and C₂₋₁₀ alkynyl;
wherein R⁵, R⁶, R^{7.3}, R^{7.4}, R^{8.1}, R^{8.2}, R^{8.3}, R^{A0}, R^{C0}, R^{D0}, R^{X}, R^{X0}, r, t and u are as defined in formula (I).

In another Embodiment (2), the invention provides a compound of Embodiment (1) or a pharmaceutically acceptable salt thereof, wherein Z¹ is C(O).

In another Embodiment (3), the invention provides a compound of Embodiment (1) or a pharmaceutically acceptable salt thereof, wherein Z¹ is N.

In another Embodiment (4), the invention provides a compound of any one of Embodiments (1)-(3) or a pharmaceutically acceptable salt thereof, wherein Z² is N.

In another Embodiment (5), the invention provides a compound of any one of Embodiments (1)-(3) or a pharmaceutically acceptable salt thereof, wherein Z² is C.

In another Embodiment, the invention provides a compound of any one of Embodiments (1)-(5) or a pharmaceutically acceptable salt thereof wherein the moiety

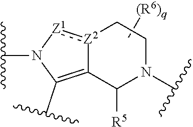

in Formula (I) is selected from

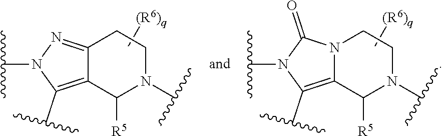

In another Embodiment (6), the invention provides a compound of any one of Embodiments (1)-(5) or a pharmaceutically acceptable salt thereof, wherein R⁵ is selected from hydrogen, halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A5}R^{B5}, and —OR^{A5}, wherein alkyl, cycloalkyl, and heterocyclyl are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1}.

In another Embodiment (7), the invention provides a compound of Embodiment (6) or a pharmaceutically acceptable salt thereof, wherein R⁵ is selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH. In another Embodiment, R⁵ is methyl.

In another Embodiment (8), the invention provides a compound of any one of Embodiments (1)-(7) or a pharmaceutically acceptable salt thereof, wherein each R⁶, at each occurrence, is selected from hydrogen, halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A6}R^{B6}, and —OR^{A6}, wherein alkyl, cycloalkyl, and heterocyclyl are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1.}

In another Embodiment (9), the invention provides a compound of Embodiment (8) or a pharmaceutically acceptable salt thereof, wherein each R⁶, at each occurrence, is selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH. In another Embodiment, R⁶ is hydrogen.

In another Embodiment (10), the invention provides a compound of any one of Embodiments (1)-(5) or a pharmaceutically acceptable salt thereof, wherein “R⁵ and R⁶” or “two of R⁶” together with the atom(s) to which they are attached form a C₃₋₁₀ cycloalkyl or heterocyclic ring of 4 to 10 members containing 1, 2 or 3 heteroatoms independently selected from oxygen, sulfur and nitrogen, wherein cycloalkyl and heterocyclic ring are each unsubstituted or substituted with at least one substituent, independently selected from R^{X6.}

In another Embodiment (11), the invention provides a compound of Embodiment (10) or a pharmaceutically acceptable salt thereof, wherein “R⁵ and R⁶” or “two of R⁶” together with the atom(s) to which they are attached form a C₃₋₈ cycloalkyl or heterocyclic ring of 4 to 8 members containing 1, 2 or 3 heteroatoms independently selected from oxygen, sulfur and nitrogen, wherein cycloalkyl and heterocyclic ring are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1}) OR^{b1.}

In another Embodiment, the invention provides a compound of any one of Embodiments (1)-(9) or a pharmaceutically acceptable salt thereof, wherein the moiety

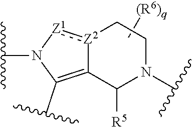

in Formula (I) is selected from

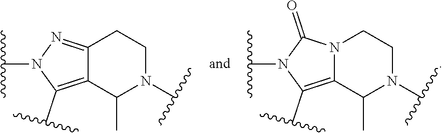 [SMILES: *c1c2c(nn1*)CCN(*)C2C ; *c1c2n(c(=O)n1*)CCN(*)C2C]

In another Embodiment, the invention provides a compound of any one of Embodiments (1)-(5) and (10)-(11) or a pharmaceutically acceptable salt thereof, wherein the moiety

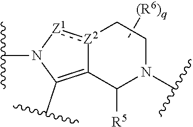

in Formula (I) is selected from

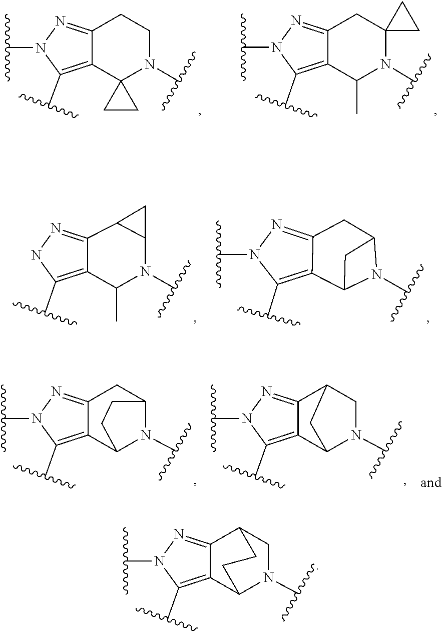

In another Embodiment (12), the invention provides a compound of any one of Embodiments (1)-(11) or a pharmaceutically acceptable salt thereof, wherein W is selected from is selected from

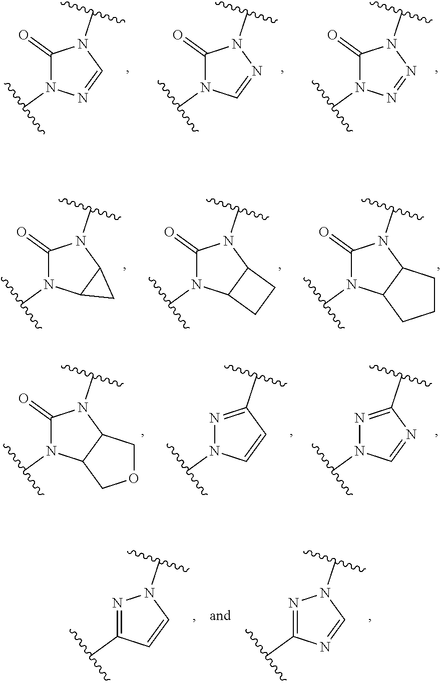

which are each unsubstituted or substituted with at least one substituent, independently selected from RX. In another Embodiment, W is selected from is selected from

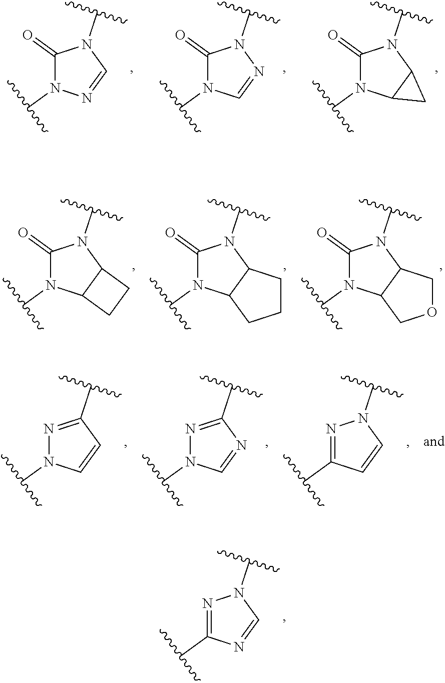

which are each unsubstituted or substituted with at least one substituent, independently selected from R^{X}.

In another Embodiment (13), the invention provides a compound of any one of Embodiments (1)-(11) or a pharmaceutically acceptable salt thereof, wherein W is

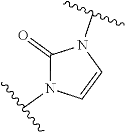 [SMILES: *n1ccn(*)c1=O]

which is unsubstituted or substituted with at least one substituent, independently selected from RX.

In another Embodiment (14), the invention provides a compound of any one of Embodiments (1)-(13) or a pharmaceutically acceptable salt thereof, wherein Q² is selected from heterocyclyl, aryl and heteroaryl.

In another Embodiment (15), the invention provides a compound of Embodiment (14) or a pharmaceutically acceptable salt thereof, wherein Q² is selected from

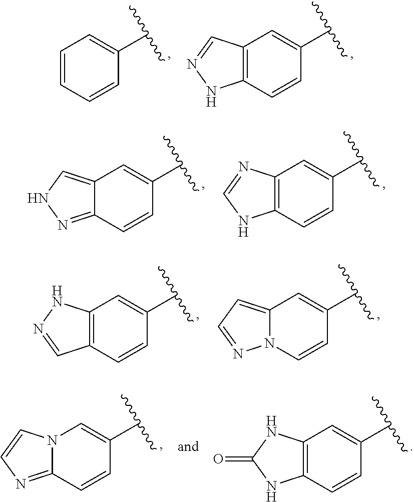

In another Embodiment (16), the invention provides a compound of any one of Embodiments (1)-(15) or a pharmaceutically acceptable salt thereof, wherein n is selected from 1, 2 and 3.

In another Embodiment (17), the invention provides a compound of any one of Embodiments (1)-(16) or a pharmaceutically acceptable salt thereof, wherein each R², at each occurrence, is independently selected from C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, NO₂, —NR^{A2}R^{B2} and —OR^{A2}, wherein alkyl, cycloalkyl and heterocyclyl aryl and heteroaryl are each unsubstituted or substituted with at least one substituent, independently selected from R^{X2.}

In another Embodiment (18), the invention provides a compound of Embodiment (17) or a pharmaceutically acceptable salt thereof, wherein each R², at each occurrence, is independently selected from hydrogen, methyl, ethyl, isopropyl, pyran, cyclopropyl, methoxy, ethoxy, F, Cl, Br, —CN, —NO₂, —NH₂, —NHCH₃, —OH

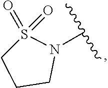 [SMILES: *N1CCCS1(=O)=O]

and wherein methyl, ethyl, isopropyl, cyclopropyl, pyran, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1}. In another Embodiment, each R², at each occurrence, is independently selected from hydrogen, methyl, ethyl, isopropyl, pyran, cyclopropyl, methoxy, ethoxy, F, Cl, Br, —CN, —NO₂, —NH₂ and OH, wherein methyl, ethyl, isopropyl, cyclopropyl, pyran, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1}), OR^{b1} In another Embodiment, each R², at each occurrence, is independently selected from F and methyl.

In another Embodiment, the invention provides a compound of any one of Embodiments (1)-(18) or a pharmaceutically acceptable salt thereof, wherein the moiety

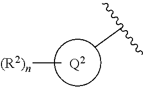

in Formula (I) is selected from

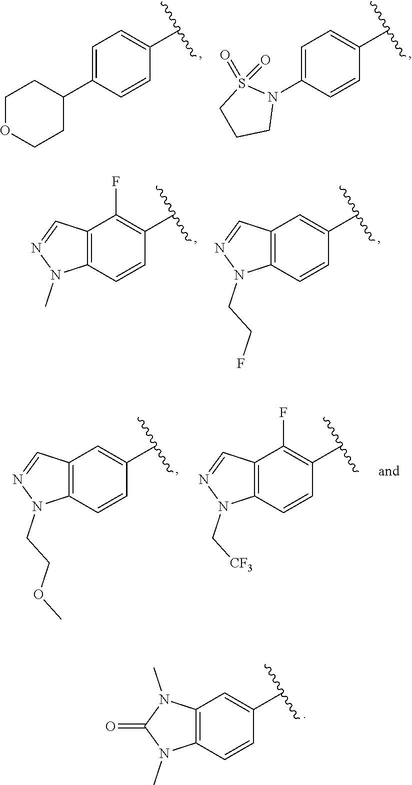

In another Embodiment, the moiety

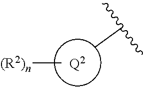

in Formula (I) is selected from

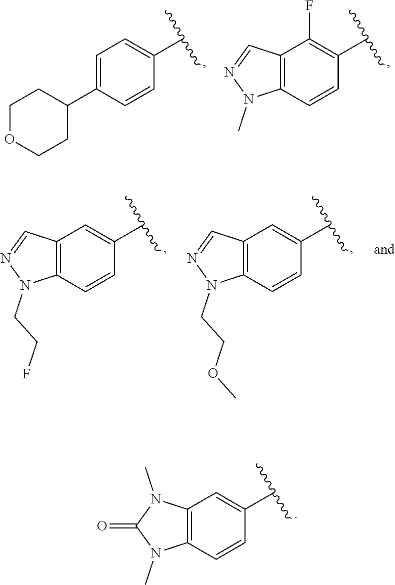 [SMILES: *c1ccc(C2CCOCC2)cc1 ; *c1ccc2c(cnn2C)c1F ; *c1ccc2c(cnn2CCF)c1 ; *c1ccc2c(cnn2CCOC)c1 ; *c1ccc2c(c1)n(C)c(=O)n2C]

In another Embodiment (19), the invention provides a compound of any one of Embodiments (1)-(18) or a pharmaceutically acceptable salt thereof, wherein Q¹ is selected from heterocyclyl, aryl and heteroaryl.

In another Embodiment (20), the invention provides a compound of Embodiment (19) or a pharmaceutically acceptable salt thereof, wherein Q¹ is selected from phenyl, pyridinyl and pyrimidinyl.

In another Embodiment (21), the invention provides a compound of any one of Embodiments (1)-(20) or a pharmaceutically acceptable salt thereof, wherein m is selected from 1, 2 and 3.

In another Embodiment (22), the invention provides a compound of any one of Embodiments (1)-(21) or a pharmaceutically acceptable salt thereof, wherein each R¹, at each occurrence, is independently selected from C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, NO₂, —NR^{A1}R^{B1} and —OR^{A1}, wherein alkyl, cycloalkyl and heterocyclyl aryl and heteroaryl are each unsubstituted or substituted with at least one substituent, independently selected from R^{X1.}

In another Embodiment (23), the invention provides a compound of Embodiment (22) or a pharmaceutically acceptable salt thereof, wherein each R¹, at each occurrence, is independently selected from hydrogen, methyl, ethyl, isopropyl, cyclopropyl, methoxy, ethoxy, F, Cl, Br, —CN, —NO₂, —NH₂ and —OH, wherein methyl, ethyl, isopropyl, cyclopropyl, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1}. In another Embodiment, each R¹, at each occurrence, is independently selected from F, methyl and cyclopropyl. In another Embodiment, each R¹, at each occurrence, is independently selected from F and methyl.

In another Embodiment, the invention provides a compound of any one of Embodiments (1)-(23) or a pharmaceutically acceptable salt thereof, wherein the moiety

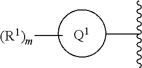

in Formula (I) is selected from

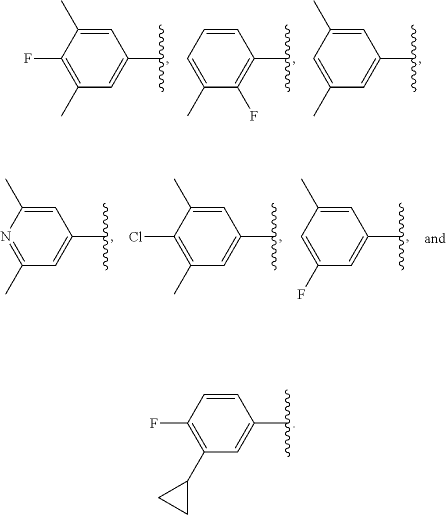

In another Embodiment (24), the invention provides a compound of any one of Embodiments (1)-(23) or a pharmaceutically acceptable salt thereof, wherein X¹ is N.

In another Embodiment (25), the invention provides a compound of any one of Embodiments (1)-(23) or a pharmaceutically acceptable salt thereof, wherein X¹ is C.

In another Embodiment (26), the invention provides a compound of any one of Embodiments (1)-(25) or a pharmaceutically acceptable salt thereof, wherein X² is N.

In another Embodiment (27), the invention provides a compound of any one of Embodiments (1)-(25) or a pharmaceutically acceptable salt thereof, wherein X² is C.

In another Embodiment (28), the invention provides a compound of any one of Embodiments (1)-(27) or a pharmaceutically acceptable salt thereof, wherein X⁵ is N.

In another Embodiment (29), the invention provides a compound of any one of Embodiments (1)-(27) or a pharmaceutically acceptable salt thereof, wherein X⁵ is C.

In another Embodiment (30), the invention provides a compound of any one of Embodiments (1)-(29) or a pharmaceutically acceptable salt thereof, wherein X⁶ is N.

In another Embodiment (31), the invention provides a compound of any one of Embodiments (1)-(29) or a pharmaceutically acceptable salt thereof, wherein X⁶ is C.

In another Embodiment (32), the invention provides a compound of any one of Embodiments (1)-(31) or a pharmaceutically acceptable salt thereof, wherein X³ is is selected from O and S.

In another Embodiment (33), the invention provides a compound of any one of Embodiments (1)-(31) or a pharmaceutically acceptable salt thereof, wherein X³ is is selected from N, NR^{7.3} and CR^{7.3.}

In another Embodiment (34), the invention provides a compound of any one of Embodiments (1)-(33) or a pharmaceutically acceptable salt thereof, wherein X⁴ is CR^{7.4.}

In another Embodiment (35), the invention provides a compound of any one of Embodiments (1)-(31) and (33) or a pharmaceutically acceptable salt thereof, wherein X⁴ is N.

In another Embodiment (36), the invention provides a compound of any one of Embodiments (1)-(31) and (33) or a pharmaceutically acceptable salt thereof, wherein X⁴ is C(O).

In another Embodiment (37), the invention provides a compound of any one of Embodiments (1)-(33) or a pharmaceutically acceptable salt thereof, wherein X⁴ is absent.

In another Embodiment (38), the invention provides a compound of any one of Embodiments (33)-(34) or a pharmaceutically acceptable salt thereof, wherein R^{7.3} and R^{7.4} are independently selected from hydrogen, halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A7}R^{B7}, and —OR^{A7}, wherein alkyl, cycloalkyl, and heterocyclyl are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1.}

In another Embodiment (39), the invention provides a compound of Embodiment (38) or a pharmaceutically acceptable salt thereof, wherein R^{7.3} and R^{7.4} are independently selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH. In another Embodiment, R^{7.3} and R^{7.4} are hydrogen.

In another Embodiment (40), the invention provides a compound of any one of Embodiments (1)-(39) or a pharmaceutically acceptable salt thereof, wherein Y¹ is N.

In another Embodiment (41), the invention provides a compound of any one of Embodiments (1)-(39) or a pharmaceutically acceptable salt thereof, wherein Y¹ is CR^{8.1.}

In another Embodiment (42), the invention provides a compound of any one of Embodiments (1)-(41) or a pharmaceutically acceptable salt thereof, wherein Y² is N.

In another Embodiment (43), the invention provides a compound of any one of Embodiments (1)-(41) or a pharmaceutically acceptable salt thereof, wherein Y² is CR^{8.2.}

In another Embodiment (44), the invention provides a compound of any one of Embodiments (1)-(43) or a pharmaceutically acceptable salt thereof, wherein Y³ is CR^{8.3.}

In another Embodiment (45), the invention provides a compound of any one of Embodiments (1)-(43) or a pharmaceutically acceptable salt thereof, wherein Y³ is N.

In another Embodiment (46), the invention provides a compound of any one of Embodiments (41) and (43)-(44) or a pharmaceutically acceptable salt thereof, wherein R^{8.1}, R^{8.2} and R^{8.3} are independently selected from are independently selected from hydrogen, halogen, C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A8}R^{B8}, and —OR^{A8}, wherein alkyl, cycloalkyl, and heterocyclyl are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1.}

In another Embodiment (47), the invention provides a compound of Embodiment (46) or a pharmaceutically acceptable salt thereof, wherein R^{8.1}, R^{8.2} and R^{8.3} are independently selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH. In another Embodiment, R^{8.1}, R^{8.2} and R^{8.3} are hydrogen.

In another Embodiment, the invention provides a compound of any one of Embodiments (1)-(36) and (38)-(47) or a pharmaceutically acceptable salt thereof, wherein the moiety

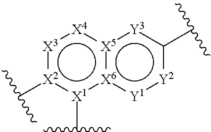

in Formula (I) is selected from

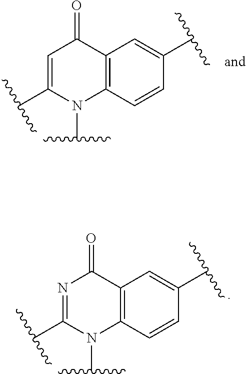 [SMILES: *c1ccc2c(c1)c(=O)cc(*)n2* ; *c1ccc2c(c1)c(=O)nc(*)n2*]

In another Embodiment, the moiety

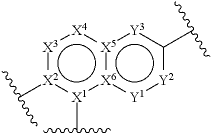

### in Formula (I) is

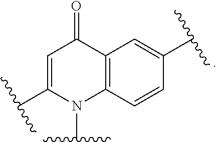 [SMILES: *c1ccc2c(c1)c(=O)cc(*)n2*]

In another Embodiment, the invention provides a compound of any one of Embodiments (1)-(33), (37) and (40)-(47) or a pharmaceutically acceptable salt thereof, wherein the moiety

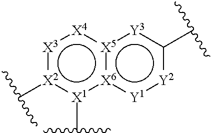

in Formula (I) is selected from

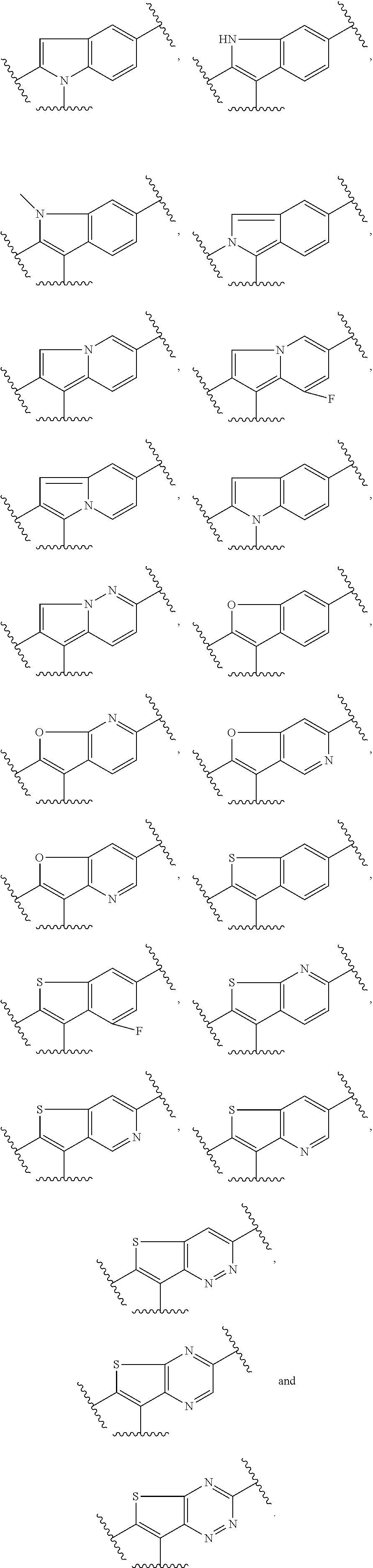

In another Embodiment, the moiety

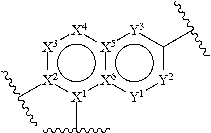

in Formula (I) is selected from

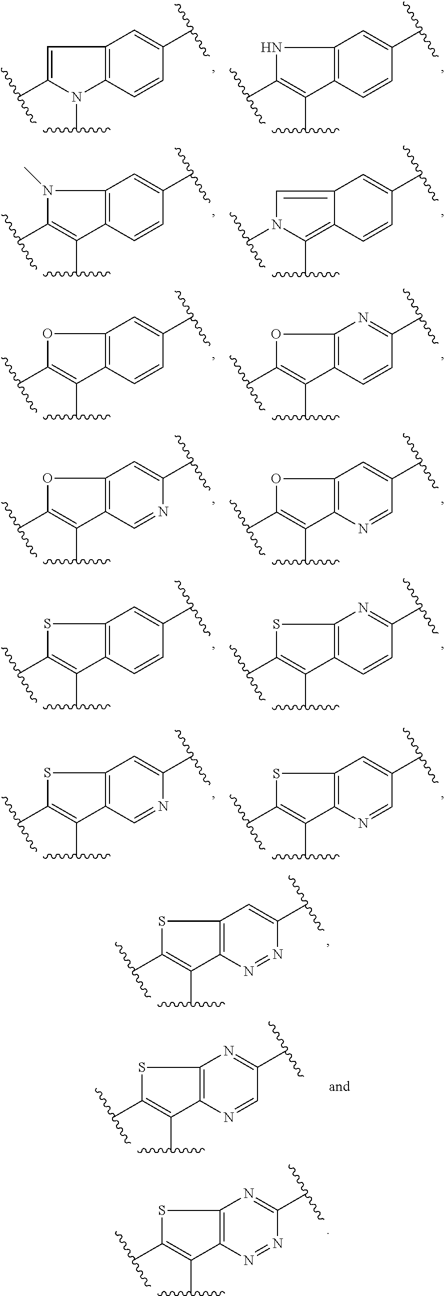

In another Embodiment, the moiety

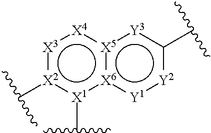

in Formula (I) is selected from

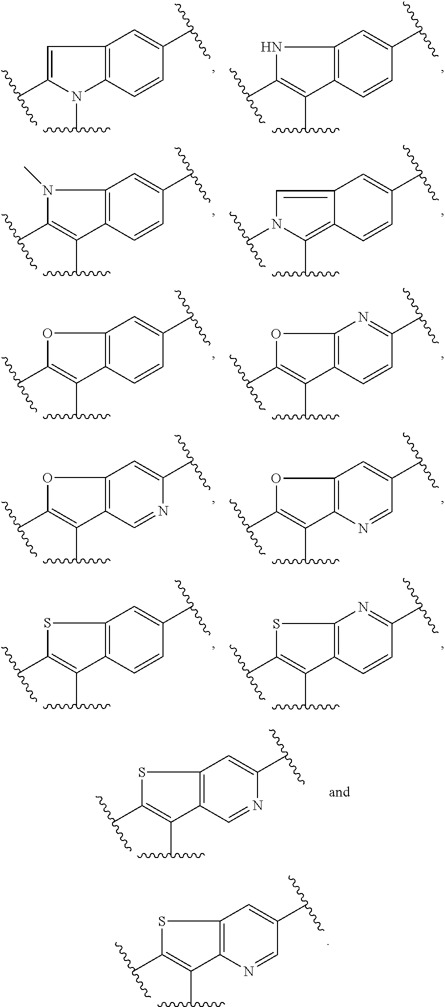

in another Embodiment the moiety

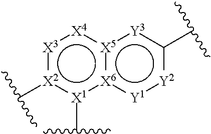

in Formula (1) is selected from

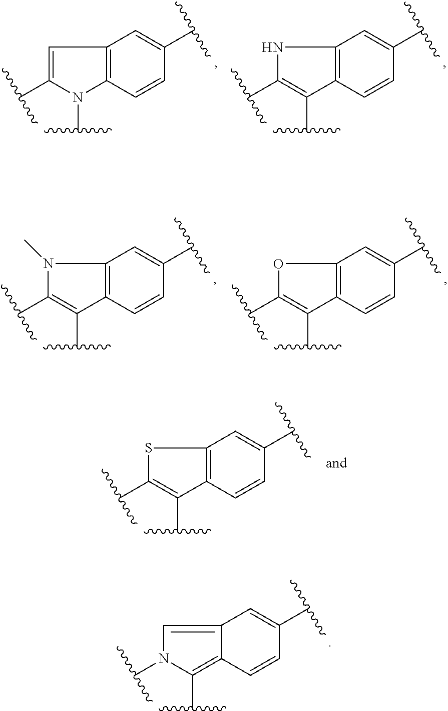 [SMILES: *c1ccc2c(c1)cc(*)n2* ; *c1ccc2c(*)c(*)[nH]c2c1 ; *c1ccc2c(*)c(*)n(C)c2c1 ; *c1ccc2c(*)c(*)oc2c1 ; *c1ccc2c(*)c(*)sc2c1 ; *c1ccc2c(*)n(*)cc2c1]

In another Embodiment (48), the invention provides a compound of any one of Embodiments (1)-(47) or a pharmaceutically acceptable salt thereof, wherein L is —CR^{C0}R^{D0}(CR^{C0}R^{D0})ᵤ—.

In another Embodiment (49), the invention provides a compound of any one of Embodiments (1)-(47) or a pharmaceutically acceptable salt thereof, wherein L is selected from (CR^{C0}R^{D0})ᵤO(CR^{C0}R^{D0})ₜ—, —(CR^{C0}R^{D0})ₜNR^{A0}(CR^{C0}R^{D0})ₜ— and —(CR^{C0}R^{D0})ᵤS(O), (CR^{C0}R^{D0})ₜ—.

In another Embodiment (50), the invention provides a compound of Embodiment (1) or a pharmaceutically acceptable salt thereof, wherein Z¹ is N, X² is C, X³ is CR^{7.3} or N, X⁴ is absent, R⁵ and R⁶ or any two of R⁶ do not form any ring, W is

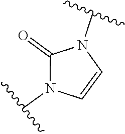 [SMILES: *n1ccn(*)c1=O]

and L is selected from —CR^{C0}R^{D0}(CR^{C0}R^{D0})ᵤ—. In another Embodiment (50′), wherein Z¹ is N, X² is C, X⁴ is absent, R⁵ and R⁶ or any two of R⁶ do not form any ring, W is

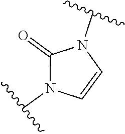 [SMILES: *n1ccn(*)c1=O]

and L is selected from —CR^{C0}R^{D0} (CR^{C0}R^{D0})ᵤ—.

In another Embodiment (51), the invention provides a compound of any one of Embodiments (1)-(50) and (50′) or a pharmaceutically acceptable salt thereof, wherein each R^{C0} and R^{D0} are independently selected from hydrogen, methyl, ethyl, isopropyl, cyclopropyl, methoxy and ethoxy, wherein methyl, ethyl, isopropyl, cyclopropyl, methoxy and ethoxy are unsubstituted or substituted with at least one substituent, independently selected from R^{X0};

or R^{C0} and R^{D0} together with the carbon atom(s) to which they are attached form C₃₋₁₀ cycloalkyl, wherein cycloalkyl is unsubstituted or substituted with at least one substituent, independently selected from R^{X0.}

In another Embodiment (52), the invention provides a compound of any one of Embodiments (1)-(49) and (51) or a pharmaceutically acceptable salt thereof, wherein R^{X0} is selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1}) NR^{a1}R^{b1}, —(CR^{c1}R^{d1})ₜR^{b1} and —(CR^{c1}R^{d1})ₜC(O)R^{a1}, wherein alkyl, alkenyl, alkynyl, cycloalkyl, heterocyclyl, aryl and heteroaryl are each unsubstituted or substituted with at least one substituent, independently selected from C₁₋₁₀ alkyl, C₃₋₁₀ cycloalkyl, halogen, CN, NO₂, —(CR^{c2}R^{d2})ₜNR^{a2}R^{b2}, —(CR^{e2}R^{d2}); OR^{b2} and —(CR^{c2}R^{d2}), C(O)R^{a2.}

In another Embodiment (53), the invention provides a compound of Embodiment (52) or a pharmaceutically acceptable salt thereof, wherein R^{X0} is selected from hydrogen, methyl, difluoromethyl, trifluoromethyl, ethyl, difluoroethyl, trifluoroethyl, isopropyl, cyclopropyl, methoxy, ethoxy, vinyl, ethynyl, F, Cl, Br, —CN, —NO₂, —NH₂ and —OH.

In another Embodiment, the invention provides a compound of any one of Embodiments (1)-(49) and (51)-(53) or a pharmaceutically acceptable salt thereof, wherein L is selected from

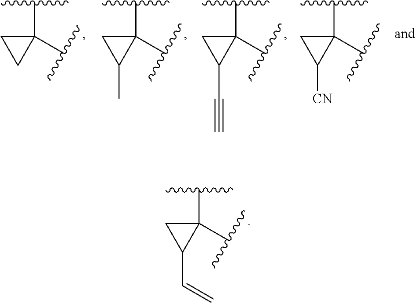

In another Embodiment, L is selected from

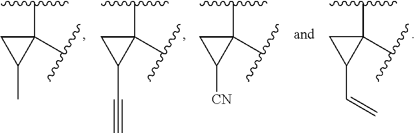

In another Embodiment (54), the invention provides a compound of any one of Embodiments (50)-(51) or a pharmaceutically acceptable salt thereof, wherein R^{X0} is selected from CN, vinyl and ethynyl.

In another Embodiment, the invention provides a compound of any one of Embodiments (50)-(51) and (54) or a pharmaceutically acceptable salt thereof, wherein L is selected from

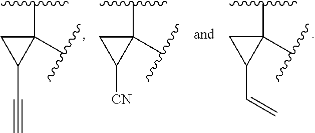

In another Embodiment (55), the invention provides a compound of any one of Embodiments (1)-(54) or a pharmaceutically acceptable salt thereof, wherein R⁴ is selected from —C(O)OH, —C(O)NHS(O)ᵣR^{A4}, —(CR^{c1}R^{d1})ₜNHS(O)ᵣR^{A4} and heteroaryl.

In another Embodiment (56), the invention provides a compound of Embodiment (55) or a pharmaceutically acceptable salt thereof, wherein R⁴ is selected from

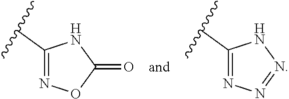 [SMILES: *c1noc(=O)[nH]1 ; *c1nnn[nH]1]

In another Embodiment (57), the invention provides a compound of any one of Embodiments (1)-(56) or a pharmaceutically acceptable salt thereof, wherein Q³ is selected from heterocyclyl, aryl and heteroaryl.

In another Embodiment (58), the invention provides a compound of Embodiment (57) or a pharmaceutically acceptable salt thereof, wherein Q³ is selected from

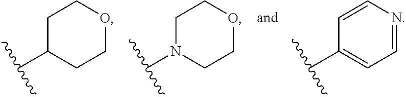

In another Embodiment (59), the invention provides a compound of any one of Embodiments (1)-(58) or a pharmaceutically acceptable salt thereof, wherein p is selected from 0, 1, 2 and 3.

In another Embodiment (60), the invention provides a compound of any one of Embodiments (1)-(59) or a pharmaceutically acceptable salt thereof, wherein each R³, at each occurrence, is independently selected from C₁₋₁₀ alkyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, NO₂, —NR^{A3}R^{B3} and —OR^{A3}, wherein alkyl, cycloalkyl and heterocyclyl aryl and heteroaryl are each unsubstituted or substituted with at least one substituent, independently selected from R^{X3.}

In another Embodiment (61), the invention provides a compound of Embodiment (60) or a pharmaceutically acceptable salt thereof, wherein each R³, at each occurrence, is independently selected from hydrogen, methyl, ethyl, isopropyl, cyclopropyl, methoxy, ethoxy, F, Cl, Br, —CN, —NO₂, —NH₂ and —OH, wherein methyl, ethyl, isopropyl, cyclopropyl, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1}. In another Embodiment, each R³, at each occurrence, is independently selected from methyl, ethyl and methoxy.

In another Embodiment, the invention provides a compound of any one of Embodiments (1)-(61) or a pharmaceutically acceptable salt thereof, wherein the moiety

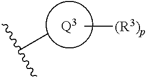

in Formula (I) is selected from

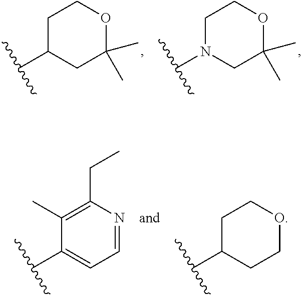

In another Embodiment (62), the invention provides a compound selected from

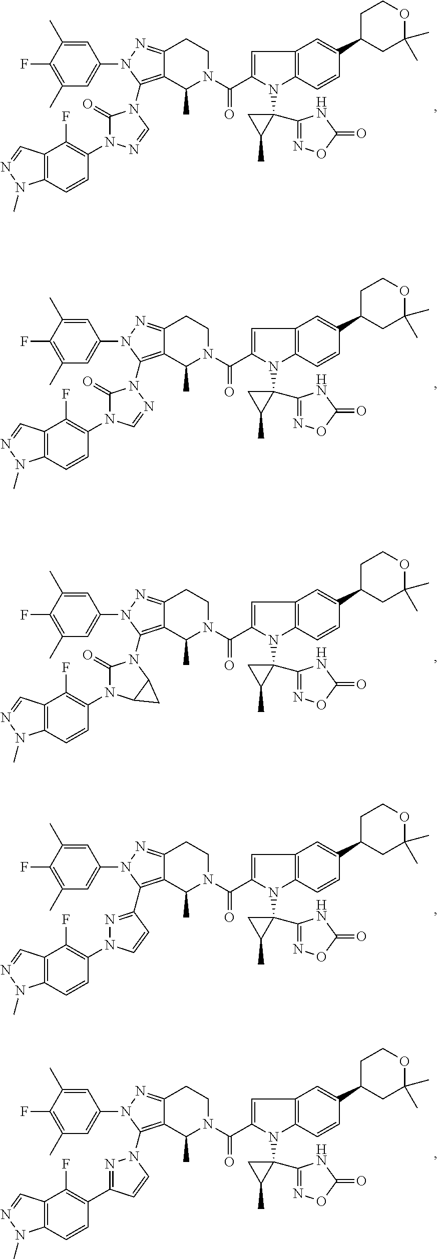

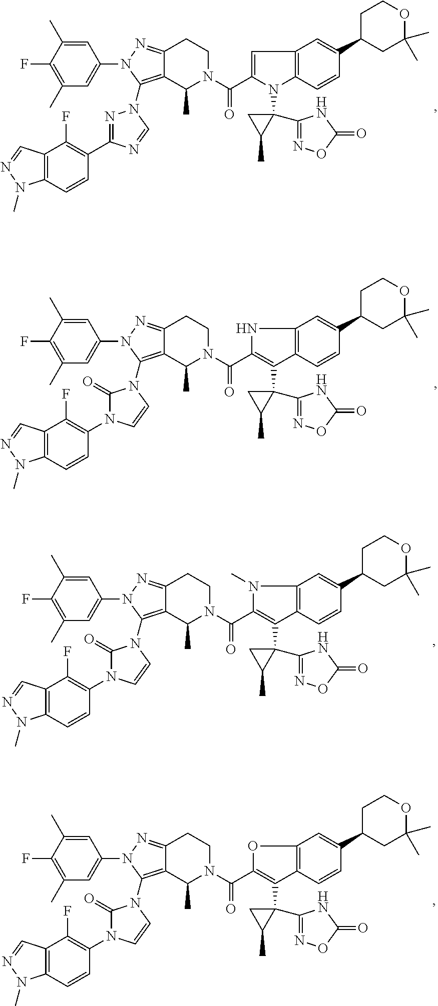

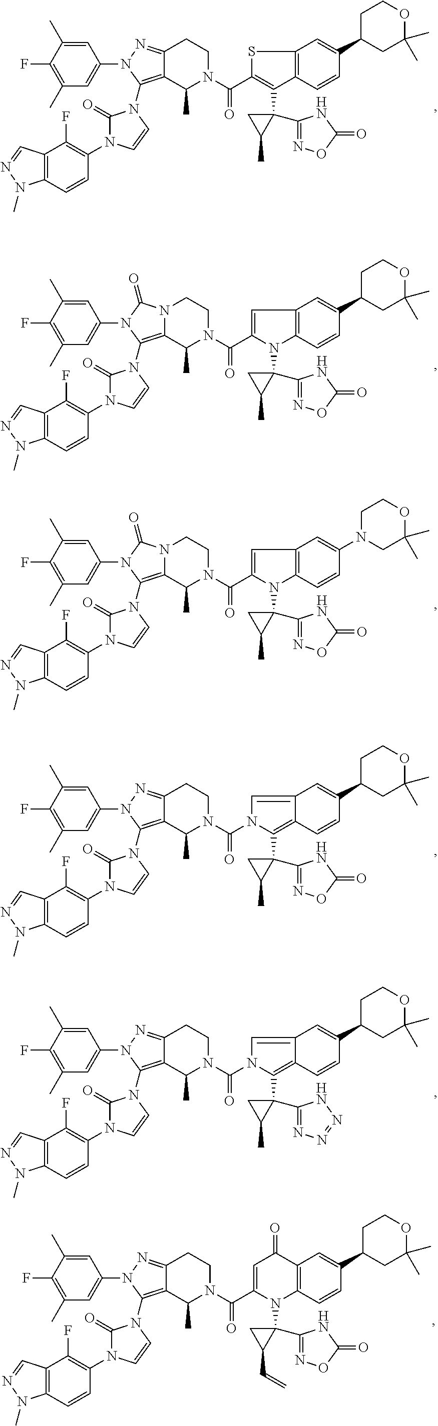

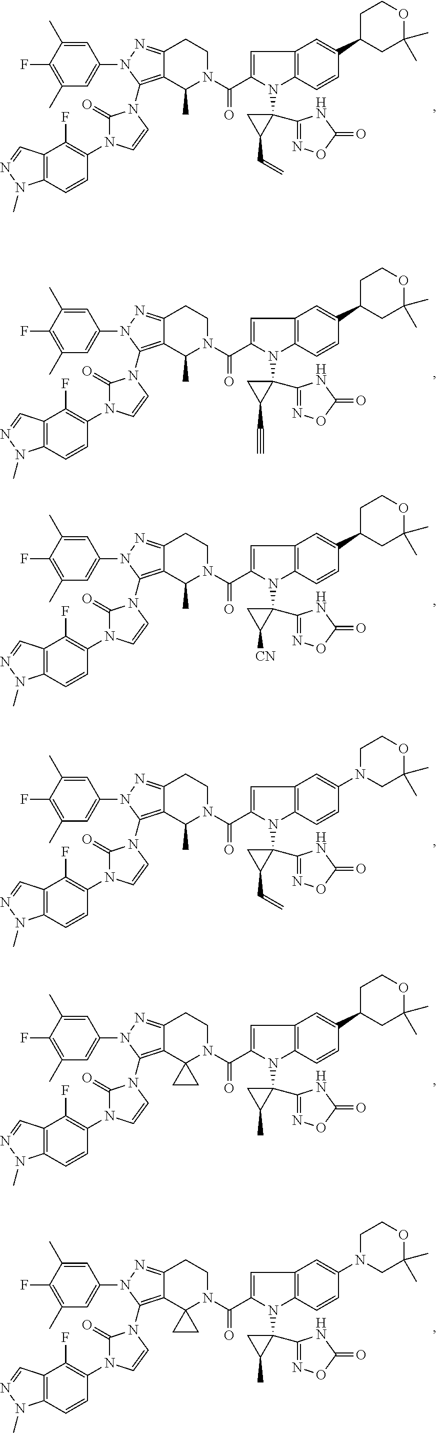

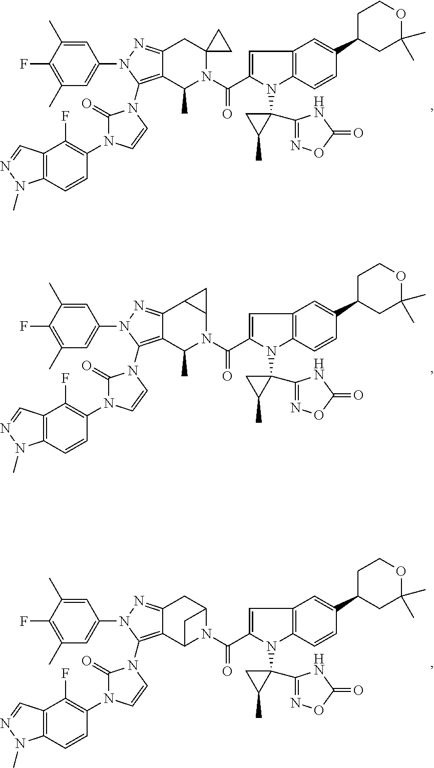 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)C2(CC2)C3)cc(C)c1F ; Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)C2CC32)cc(C)c1F ; Cc1cc(-n2nc3c(c2-n2ccn(-c4ccc5c(cnn5C)c4F)c2=O)C2CC(C3)N2C(=O)c2cc3cc([C@H]4CCOC(C)(C)C4)ccc3n2[C@@]2(c3noc(=O)[nH]3)C[C@@H]2C)cc(C)c1F]

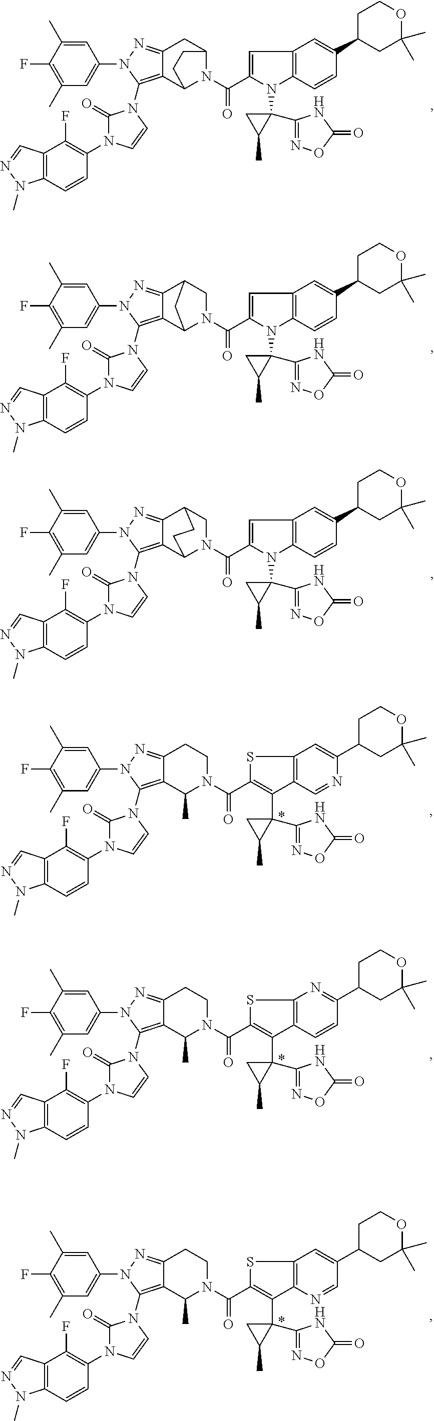

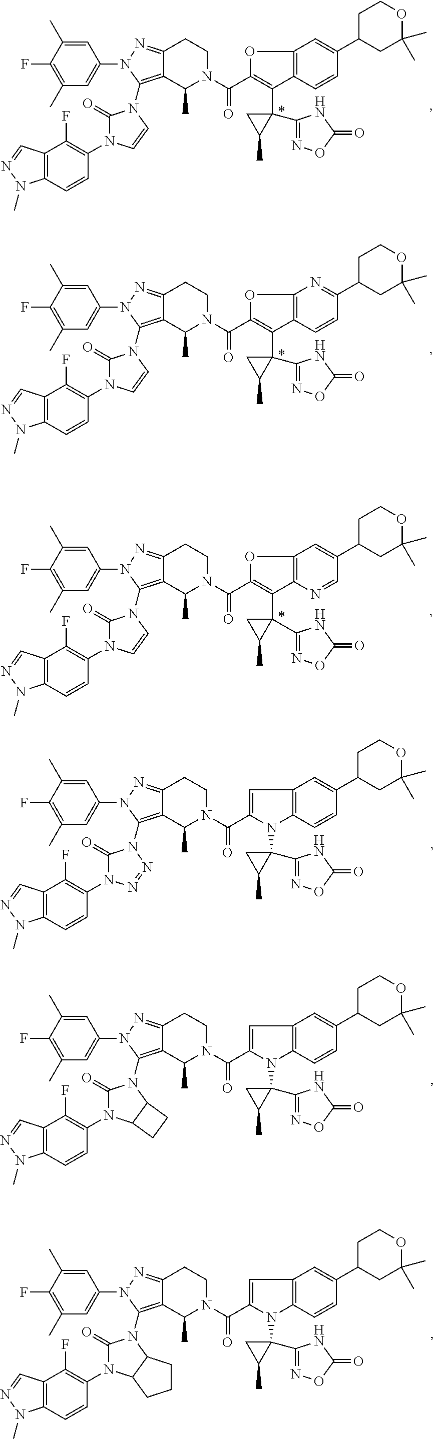

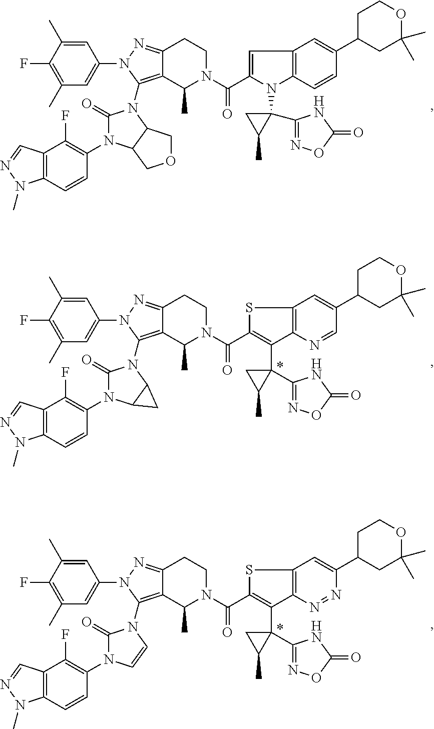 [SMILES: Cc1cc(-n2nc3c(c2N2C(=O)N(c4ccc5c(cnn5C)c4F)C4COCC42)[C@H](C)N(C(=O)c2cc4cc(C5CCOC(C)(C)C5)ccc4n2[C@@]2(c4noc(=O)[nH]4)C[C@@H]2C)CC3)cc(C)c1F ; Cc1cc(-n2nc3c(c2N2C(=O)N(c4ccc5c(cnn5C)c4F)C4CC42)[C@H](C)N(C(=O)c2sc4cc(C5CCOC(C)(C)C5)cnc4c2C2(c4noc(=O)[nH]4)C[C@@H]2C)CC3)cc(C)c1F ; Cc1cc(-n2nc3c(c2-n2ccn(-c4ccc5c(cnn5C)c4F)c2=O)[C@H](C)N(C(=O)c2sc4cc(C5CCOC(C)(C)C5)nnc4c2C2(c4noc(=O)[nH]4)C[C@@H]2C)CC3)cc(C)c1F]

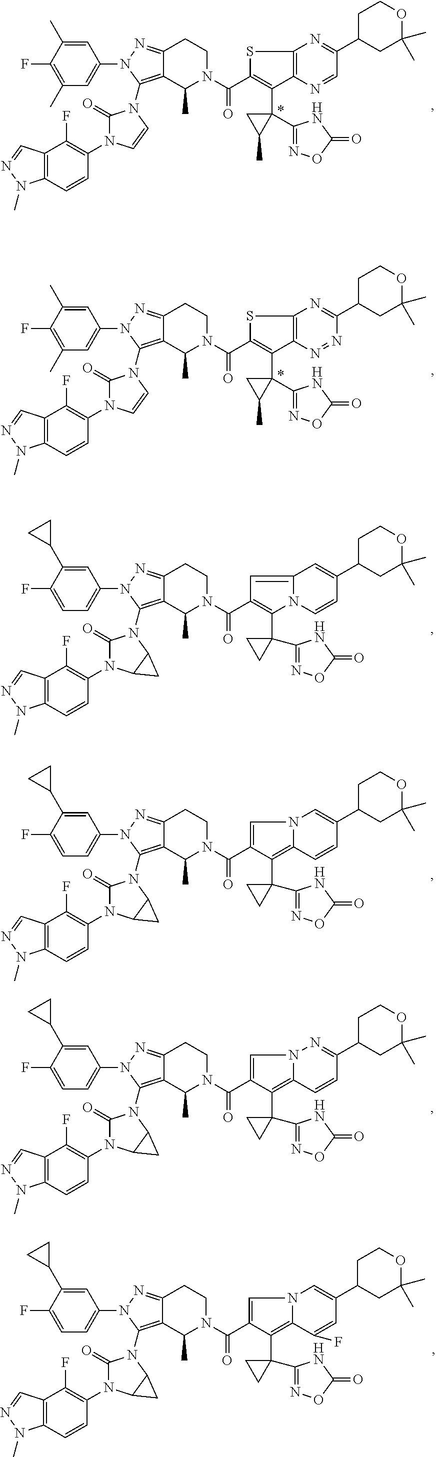

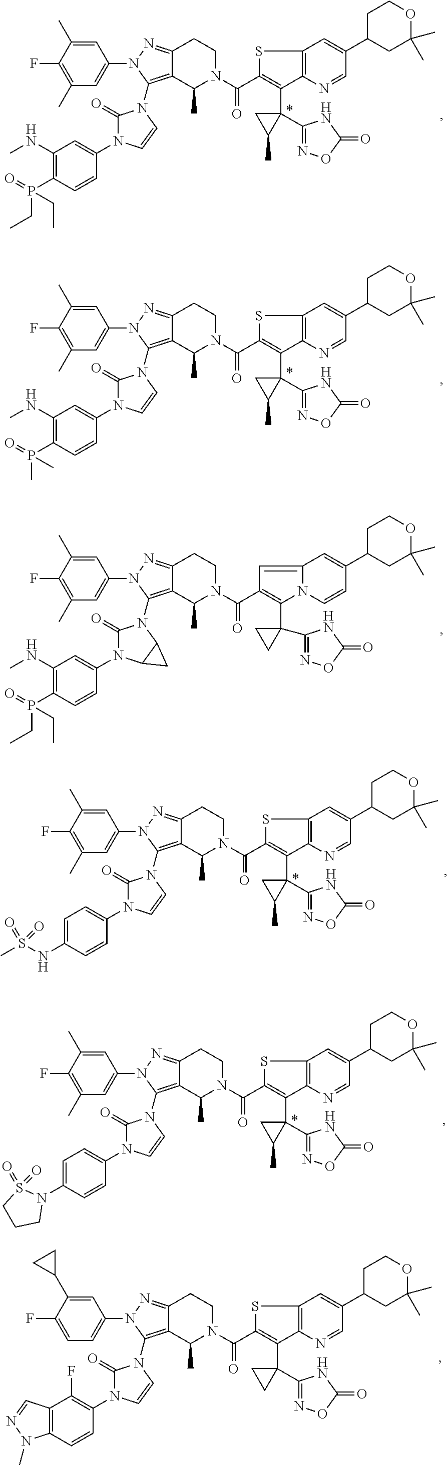

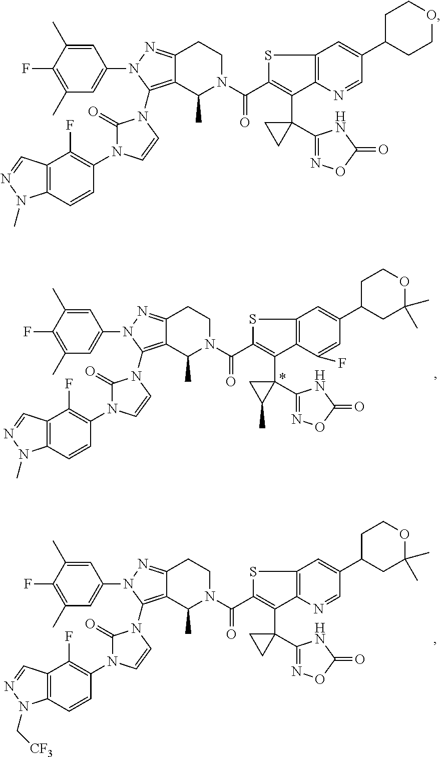 [SMILES: Cc1cc(-n2nc3c(c2-n2ccn(-c4ccc5c(cnn5C)c4F)c2=O)[C@H](C)N(C(=O)c2sc4cc(C5CCOCC5)cnc4c2C2(c4noc(=O)[nH]4)CC2)CC3)cc(C)c1F ; Cc1cc(-n2nc3c(c2-n2ccn(-c4ccc5c(cnn5C)c4F)c2=O)[C@H](C)N(C(=O)c2sc4cc(C5CCOC(C)(C)C5)cc(F)c4c2C2(c4noc(=O)[nH]4)C[C@@H]2C)CC3)cc(C)c1F ; Cc1cc(-n2nc3c(c2-n2ccn(-c4ccc5c(cnn5CC(F)(F)F)c4F)c2=O)[C@H](C)N(C(=O)c2sc4cc(C5CCOC(C)(C)C5)cnc4c2C2(c4noc(=O)[nH]4)CC2)CC3)cc(C)c1F]

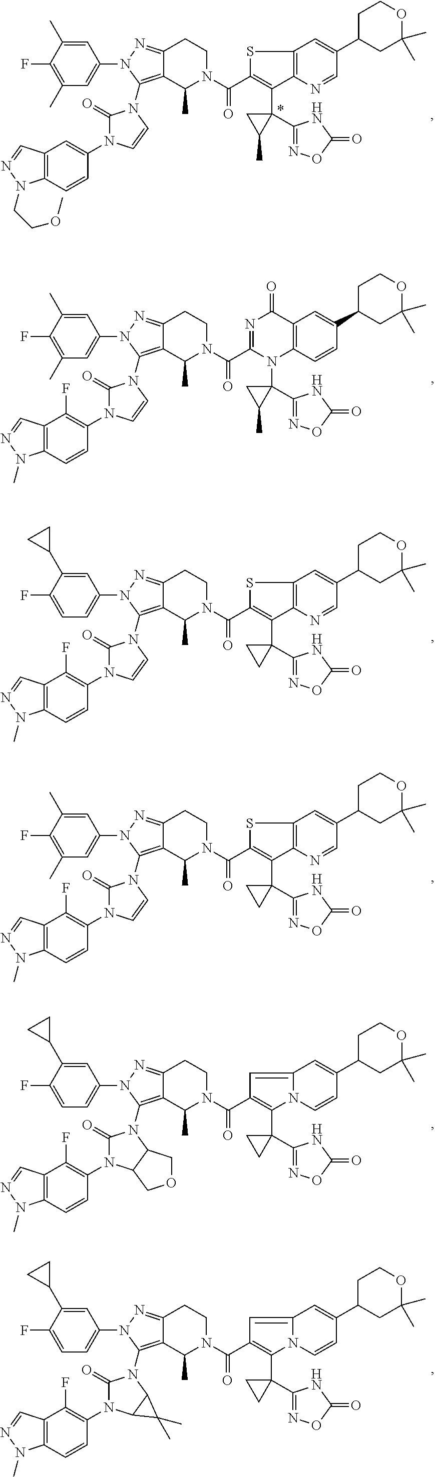

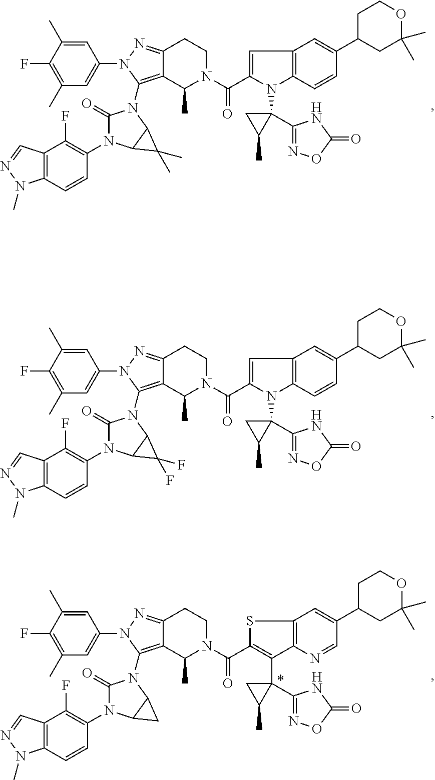 [SMILES: Cc1cc(-n2nc3c(c2N2C(=O)N(c4ccc5c(cnn5C)c4F)C4C2C4(C)C)[C@H](C)N(C(=O)c2cc4cc(C5CCOC(C)(C)C5)ccc4n2[C@@]2(c4noc(=O)[nH]4)C[C@@H]2C)CC3)cc(C)c1F ; Cc1cc(-n2nc3c(c2N2C(=O)N(c4ccc5c(cnn5C)c4F)C4C2C4(F)F)[C@H](C)N(C(=O)c2cc4cc(C5CCOC(C)(C)C5)ccc4n2[C@@]2(c4noc(=O)[nH]4)C[C@@H]2C)CC3)cc(C)c1F ; Cc1cc(-n2nc3c(c2N2C(=O)N(c4ccc5c(cnn5C)c4F)C4CC42)[C@H](C)N(C(=O)c2sc4cc(C5CCOC(C)(C)C5)cnc4c2C2(c4noc(=O)[nH]4)C[C@@H]2C)CC3)cc(C)c1F]

and pharmaceutically acceptable salts thereof.

In another Embodiment (63), the invention provides a pharmaceutical composition comprising a compound of any one of Embodiments (1)-(62) or a pharmaceutically acceptable salt thereof and at least one pharmaceutically acceptable carrier.

In another Embodiment (64), the invention provides a method of treating, ameliorating or preventing a condition, which responds to inhibition of GLP-1R, comprising administering to a subject in need of such treatment an effective amount of a compound of any one of Embodiments (1)-(62), or a pharmaceutically acceptable salt thereof, or a pharmaceutical composition thereof, and optionally in combination with a second therapeutic agent.

In another Embodiment (65), the invention provides a use of a compound of any one of Embodiments (1)-(62) or a pharmaceutically acceptable salt thereof in the preparation of a medicament for treating or preventing a GLP-1 receptor-mediated disease or disorder or of modulating GLP-1 receptor.

In another Embodiment (66), the invention provides the use of Embodiment (65), wherein the disease or disorder is includes but not limited to, diabetes, diabetic complication, obesity, impaired glucose tolerance, overweight condition, hyperlipidemia, hypercholesteremia, atherosclerosis, hypertension, coronary heart disease, congestive heart failure, cardiac arrhythmias, brain infarction, stroke, liver disease, nonalcoholic fatty liver disease (NAFLD), nonalcoholic steatohepatitis (NASH), dementia, Parkinson's disease, and diabetic kidney disease.

In yet another of its aspects, there is provided a kit comprising a compound disclosed herein, or a pharmaceutically acceptable salt thereof; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound disclosed herein, or a pharmaceutically acceptable salt thereof; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound disclosed herein, or a pharmaceutically acceptable salt thereof.

In another of its aspects, there is provided a method of inhibiting a GLP-1R comprising contacting the GLP-1R with a compound disclosed herein, or a pharmaceutically acceptable salt thereof.

In yet another of its aspects, there is provided a method of inhibiting a GLP-1R comprising causing a compound disclosed herein, or a pharmaceutically acceptable salt thereof to be present in a subject in order to inhibit the GLP-1R in vivo.

In a further of its aspects, there is provided a method of inhibiting GLP-1R comprising administering a first compound to a subject that is converted in vivo to a second compound wherein the second compound inhibits the GLP-1R in vivo, the second compound being a compound according to any one of the above embodiments and variations.

In another of its aspects, there is provided a method of treating a disease state for which a GLP-1R possesses activity that contributes to the pathology and/or symptomology of the disease state, the method comprising causing a compound disclosed herein, or a pharmaceutically acceptable salt thereof to be present in a subject in a therapeutically effective amount for the disease state.

In a further of its aspects, there is provided a method of treating a disease state for which a GLP-1R possesses activity that contributes to the pathology and/or symptomology of the disease state, the method comprising administering a first compound to a subject that is converted in vivo to a second compound wherein the second compound inhibits the GLP-1R in vivo. It is noted that the compounds of the present invention may be the first or second compounds.

In another of its aspects, there is provided a method of treating a disease state for which a mutation in the GLP-1R gene contributes to the pathology and/or symptomology of the disease state is includes but not limited to, diabetes, diabetic complication, obesity, impaired glucose tolerance, overweight condition, hyperlipidemia, hypercholesteremia, atherosclerosis, hypertension, coronary heart disease, congestive heart failure, cardiac arrhythmias, brain infarction, stroke, liver disease, nonalcoholic fatty liver disease (NAFLD), nonalcoholic steatohepatitis (NASH), dementia, Parkinson's disease, and diabetic kidney disease.

In still another of its aspects, the present invention relates to the use of a compound of any of the above embodiments and variations as a medicament. In yet another of its aspects, the present invention relates to the use of a compound according to any one of the above embodiments and variations in the manufacture of a medicament for inhibiting a GLP-1R.

In a further of its aspects, the present invention relates to the use of a compound according to any one of the above embodiments and variations in the manufacture of a medicament for treating a disease state for which a GLP-1R possesses activity that contributes to the pathology and/or symptomology of the disease state.

### Administration and Pharmaceutical Compositions

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors known to those of ordinary skill in the art. For example, for the treatment of neoplastic diseases and immune system disorders, the required dosage will also vary depending on the mode of administration, the particular condition to be treated and the effect desired.

In general, satisfactory results are indicated to be obtained systemically at daily dosages of from about 0.001 to about 100 mg/kg per body weight, or particularly, from about 0.03 to 2.5 mg/kg per body weight. An indicated daily dosage in the larger mammal, e.g. humans, may be in the range from about 0.5 mg to about 2000 mg, or more particularly, from about 0.5 mg to about 1000 mg, conveniently administered, for example, in divided doses up to four times a day or in retard form. Suitable unit dosage forms for oral administration comprise from ca. 1 to 50 mg active ingredient.

Compounds of the disclosure may be administered as pharmaceutical compositions by any conventional route; for example, enterally, e.g., orally, e.g., in the form of tablets or capsules; parenterally, e.g., in the form of injectable solutions or suspensions; or topically, e.g., in the form of lotions, gels, ointments or creams, or in a nasal or suppository form.

Pharmaceutical compositions comprising a compound of the present disclosure in free form or in a pharmaceutically acceptable salt form in association with at least one pharmaceutically acceptable carrier or diluent may be manufactured in a conventional manner by mixing, granulating, coating, dissolving or lyophilizing processes. For example, pharmaceutical compositions comprising a compound of the disclosure in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

In one embodiment, the pharmaceutical compositions are solutions of the active ingredient, including suspensions or dispersions, such as isotonic aqueous solutions. In the case of lyophilized compositions comprising the active ingredient alone or together with a carrier such as mannitol, dispersions or suspensions can be made up before use. The pharmaceutical compositions may be sterilized and/or contain adjuvants, such as preserving, stabilizing, wetting or emulsifying agents, solution promoters, salts for regulating the osmotic pressure and/or buffers. Suitable preservatives include but are not limited to antioxidants such as ascorbic acid, or microbicides, such as sorbic acid or benzoic acid. The solutions or suspensions may further comprise viscosity-increasing agents, including but not limited to, sodium carboxymethylcellulose, carboxymethylcellulose, dextran, polyvinylpyrrolidone, gelatins, or solubilizers, e.g. Tween 80 (polyoxyethylene (20) sorbitan monooleate).

Suspensions in oil may comprise as the oil component the vegetable, synthetic, or semi-synthetic oils customary for injection purposes. Examples include but are not limited to liquid fatty acid esters that contain as the acid component a long-chained fatty acid having 8-22 carbon atoms, or in some embodiments, 12-22 carbon atoms. Suitable liquid fatty acid esters include but are not limited to lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid and linoleic acid, and if desired, may contain antioxidants, for example vitamin E, 3-carotene or 3,5-di-tert-butyl-hydroxytoluene. The alcohol component of these fatty acid esters may have six carbon atoms and may be monovalent or polyvalent, for example a mono-, di- or trivalent, alcohol. Suitable alcohol components include but are not limited to methanol, ethanol, propanol, butanol or pentanol or isomers thereof; glycol and glycerol.

Other suitable fatty acid esters include but are not limited ethyl-oleate, isopropyl myristate, isopropyl palmitate, LABRAFIL® M 2375, (polyoxyethylene glycerol), LABRAFIL® M 1944 CS (unsaturated polyglycolized glycerides prepared by alcoholysis of apricot kernel oil and comprising glycerides and polyethylene glycol ester), LABRASOL™ (saturated polyglycolized glycerides prepared by alcoholysis of TCM and comprising glycerides and polyethylene glycol ester; all available from GaKefosse, France), and/or MIGLYOL® 812 (triglyceride of saturated fatty acids of chain length C8 to C12 from Hüls AG, Germany), and vegetable oils such as cottonseed oil, almond oil, olive oil, castor oil, sesame oil, soybean oil, or groundnut oil.

Pharmaceutical compositions for oral administration may be obtained, for example, by combining the active ingredient with one or more solid carriers, and if desired, granulating a resulting mixture, and processing the mixture or granules by the inclusion of additional excipients, to form tablets or tablet cores.

Suitable carriers include but are not limited to fillers, such as sugars, for example lactose, saccharose, mannitol or sorbitol, cellulose preparations and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, carboxymethyl starch, crosslinked polyvinylpyrrolidone, alginic acid or a salt thereof, such as sodium alginate. Additional excipients include but are not limited to flow conditioners and lubricants, for example silicic acid, talc, stearic acid or salts thereof, such as magnesium or calcium stearate, and/or polyethylene glycol, or derivatives thereof.

Tablet cores may be provided with suitable, optionally enteric, coatings through the use of, inter alia, concentrated sugar solutions which may comprise gum arable, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as cellulose acetate phthalate or hydroxypropyl methylcellulose phthalate. Dyes or pigments may be added to the tablets or tablet coatings, for example for identification purposes or to indicate different doses of active ingredient.

Pharmaceutical compositions for oral administration may also include hard capsules comprising gelatin or soft-sealed capsules comprising gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient may be dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories comprising a combination of the active ingredient and a suppository base. Suitable suppository bases are, for example, natural or synthetic triglycerides, paraffin hydrocarbons, polyethylene glycols or higher alkanols.

Pharmaceutical compositions suitable for parenteral administration may comprise aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers. The active ingredient, optionally together with excipients, can also be in the form of a lyophilizate and can be made into a solution before parenteral administration by the addition of suitable solvents. Solutions such as are used, for example, for parenteral administration can also be employed as infusion solutions. The manufacture of injectable preparations is usually carried out under sterile conditions, as is the filling, for example, into ampoules or vials, and the sealing of the containers.

The disclosure also provides for a pharmaceutical combination, e.g. a kit, comprising a) a first agent which is a compound of the disclosure as disclosed herein, in free form or in pharmaceutically acceptable salt form, and b) at least one co-agent. The kit can comprise instructions for its administration.

### Combination Therapies

The compounds or pharmaceutical acceptable salts of the disclosure may be administered as the sole therapy, or together with other therapeutic agent or agents.

For example, the therapeutic effectiveness of one of the compounds described herein may be enhanced by administration of an adjuvant (i.e. by itself the adjuvant may only have minimal therapeutic benefit, but in combination with another therapeutic agent, the overall therapeutic benefit to the individual is enhanced). Or, by way of example only, the benefit experienced by an individual may be increased by administering one of the compounds described herein with another therapeutic agent that also has therapeutic benefit. By way of example only, in a treatment for gout involving administration of one of the compounds described herein, increased therapeutic benefit may result by also providing the individual with another therapeutic agent for gout. Or, by way of example only, if one of the side effects experienced by an individual upon receiving one of the compounds described herein is nausea, then it may be appropriate to administer an anti-nausea agent in combination with the compound. Or, the additional therapy or therapies include, but are not limited to physiotherapy, psychotherapy, radiation therapy, application of compresses to a diseased area, rest, altered diet, and the like. Regardless of the disease, disorder or condition being treated, the overall benefit experienced by the individual may be additive of the two therapies or the individual may experience a synergistic benefit.

In the instances where the compounds described herein are administered in combination with other therapeutic agents, the compounds described herein may be administered in the same pharmaceutical composition as other therapeutic agents, or because of different physical and chemical characteristics, be administered by a different route. For example, the compounds described herein may be administered orally to generate and maintain good blood levels thereof, while the other therapeutic agent may be administered intravenously. Thus the compounds described herein may be administered concurrently, sequentially or dosed separately to other therapeutic agents.

### EXAMPLES

Various methods may be developed for synthesizing a compound of formula (I) or a pharmaceutically acceptable salt thereof. Representative methods for synthesizing a compound of formula (I) or a pharmaceutically acceptable salt thereof are provided in the Examples. It is noted, however, that a compound of formula (I) or a pharmaceutically acceptable salt thereof may also be synthesized by other synthetic routes that others may devise.

It will be readily recognized that certain compounds of formula (I) have atoms with linkages to other atoms that confer a particular stereochemistry to the compound (e.g., chiral centers). It is recognized that synthesis of a compound of formula (I) or a pharmaceutically acceptable salt thereof may result in the creation of mixtures of different stereoisomers (enantiomers, diastereomers). Unless a particular stereochemistry is specified, recitation of a compound is intended to encompass all of the different possible stereoisomers.

A compound of formula (I) can also be prepared as a pharmaceutically acceptable acid addition salt by, for example, reacting the free base form of the at least one compound with a pharmaceutically acceptable inorganic or organic acid. Alternatively, a pharmaceutically acceptable base addition salt of the at least one compound of formula (I) can be prepared by, for example, reacting the free acid form of the at least one compound with a pharmaceutically acceptable inorganic or organic base. Inorganic and organic acids and bases suitable for the preparation of the pharmaceutically acceptable salts of compounds of formula (I) are set forth in the definitions section of this Application. Alternatively, the salt forms of the compounds of formula (I) can be prepared using salts of the starting materials or intermediates.

The free acid or free base forms of the compounds of formula (I) can be prepared from the corresponding base addition salt or acid addition salt form. For example, a compound of formula (I) in an acid addition salt form can be converted to the corresponding free base thereof by treating with a suitable base (e.g., ammonium hydroxide solution, sodium hydroxide, and the like). A compound of formula (I) in a base addition salt form can be converted to the corresponding free acid thereof by, for example, treating with a suitable acid (e.g., hydrochloric acid, etc).

The N-oxides of a compound of formula (I) or a pharmaceutically acceptable salt thereof can be prepared by methods known to those of ordinary skill in the art. For example, N-oxides can be prepared by treating an unoxidized form of the compound of formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0 to 80° C. Alternatively, the N-oxides of the compounds of formula (I) can be prepared from the N-oxide of an appropriate starting material.

Compounds of formula (I) in an unoxidized form can be prepared from N-oxides of compounds of formula (I) by, for example, treating with a reducing agent (e.g., sulfur, sulfur dioxide, triphenyl phosphine, lithium borohydride, sodium borohydride, phosphorus trichloride, tribromide, and the like) in an suitable inert organic solvent (e.g., acetonitrile, ethanol, aqueous dioxane, and the like) at 0 to 80° C.

Protected derivatives of the compounds of formula (I) can be made by methods known to those of ordinary skill in the art. A detailed description of the techniques applicable to the creation of protecting groups and their removal can be found in T. W. Greene, Protecting Groups in Organic Synthesis, 3rd edition, John Wiley & Sons, Inc. 1999.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. For example, the following abbreviations may be used in the examples and throughout the specification: g (grams); mg (milligrams); L (liters); mL (milliliters); μL (microliters); psi (pounds per square inch); M (molar); mM (millimolar); i.v. (intravenous); Hz (Hertz); MHz (megahertz); mol (moles); mmol (millimoles); RT (room temperature); min (minutes); h (hours); mp (melting point); TLC (thin layer chromatography); Rt (retention time); RP (reverse phase); MeOH (methanol); i-PrOH (isopropanol); TEA (triethylamine); TFA (trifluoroacetic acid); TFAA (trifluoroacetic anhydride); THF (tetrahydrofuran); DMSO (dimethyl sulfoxide); EtOAc (ethyl acetate); DME (1,2-dimethoxyethane); DCM (dichloromethane); DCE (dichloroethane); DMF (N,N-dimethylformamide); DMPU (N,N′-dimethylpropyleneurea); CDI (1,1-carbonyldiimidazole); IBCF (isobutyl chloroformate); HOAc (acetic acid); HOSu (N-hydroxysuccinimide); HOBT (1-hydroxybenzotriazole); Et₂O (diethyl ether); EDCI (1-(3-dimethylaminopropyl)-3-ethylcarbodiimide hydrochloride); BOC (tert-butyloxycarbonyl); FMOC (9-fluorenylmethoxycarbonyl); DCC (dicyclohexylcarbodiimide); CBZ (benzyloxycarbonyl); Ac (acetyl); atm (atmosphere); TMSE (2-(trimethylsilyl)ethyl); TMS (trimethylsilyl); TIPS (triisopropylsilyl); TBS (t-butyldimethylsilyl); DMAP (4-dimethylaminopyridine); Me (methyl); OMe (methoxy); Et (ethyl); tBu (tert-butyl); HPLC (high pressure liquid chromatography); BOP (bis(2-oxo-3-oxazolidinyl)phosphinic chloride); TBAF (tetra-n-butylammonium fluoride); m-CPBA (meta-chloroperbenzoic acid).

For example, the following abbreviations in table 1 may be used in the examples and throughout the specification.

References to ether or Et₂O are to diethyl ether; brine refers to a saturated aqueous solution of NaCl. Unless otherwise indicated, all temperatures are expressed in ° C. (degrees Centigrade). All reactions were conducted under an inert atmosphere at RT unless otherwise noted.

1H NMR spectra were recorded on a Varian Mercury Plus 400. Chemical shifts are expressed in parts per million (ppm). Coupling constants are in units of hertz (Hz). Splitting patterns describe apparent multiplicities and are designated as s (singlet), d (doublet), t (triplet), q (quartet), m (multiplet) and br (broad).

Low-resolution mass spectra (MS) and compound purity data were acquired on a Shimadzu LC/MS single quadrupole system equipped with electrospray ionization (ESI) source, UV detector (220 and 254 nm), and evaporative light scattering detector (ELSD). Thin-layer chromatography was performed on 0.25 mm Superchemgroup silica gel plates (60F-254), visualized with UV light, 5% ethanolic phosphomolybdic acid, ninhydrin, or p-anisaldehyde solution. Flash column chromatography was performed on silica gel (200-300 mesh, Branch of Qingdao Haiyang Chemical Co., Ltd).

### Synthetic Schemes

Synthetic methods for preparing the compounds of the present invention are illustrated in the following Schemes and Examples. Starting materials are commercially available or may be made according to procedures known in the art or as illustrated herein.

As shown in Scheme 1, the compounds of formula I can be synthesized from intermediates of formula II and III, which are either known in the literature or may be prepared by a variety of methods familiar to those skilled in the art. Coupling of III and II can be readily achieved under standard amide bond forming conditions to provide compounds of formula I.

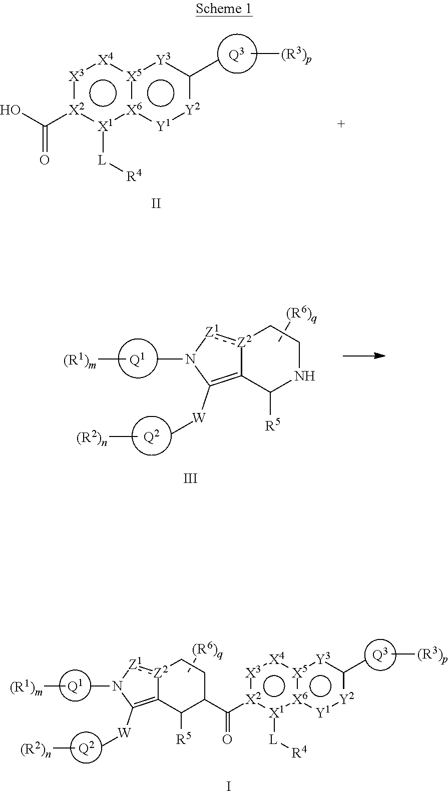

As an illustration of the preparation of intermediates of formula II, a preparation of compound IIa is shown in Scheme 2. Compound IIa-A which are commercially available or made according to procedures known in the art or as illustrated herein can be reacted with IIa-B via metal catalyzed cross coupling to obtain compound IIa-C. Compound IIa-C halogenate to Compound IIa-D. The cyanomethylation from IIa-D to Ila-E can be prepared from side-chain-substituted arenes by functional group transformation. The cycloalkane ring formed from IIa-E to IIa-F. Treatment of compound IIa-F with hydroxyamine followed by reaction with CDI and hydrolyzed to afford compound IIa.

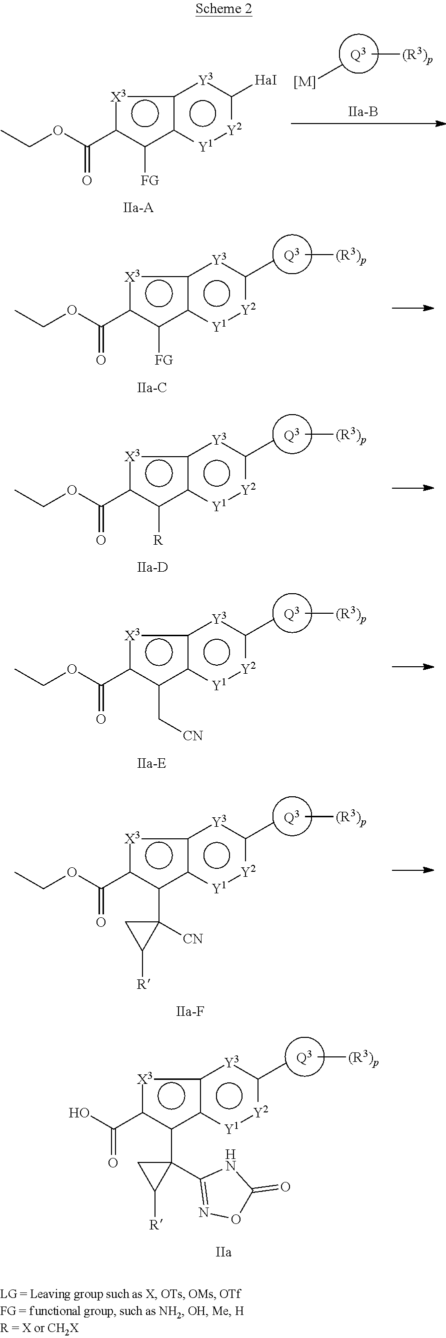

In some cases, the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Examples 1

### 3-(2S)-1-(6-(2,2-dimethyltetrahydro-2H-pyran-4-yl)-2-(S)-3-(3-(4-fluoro-1-methy 1-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1-methyl-1H-indol-3-yl)-2-methylcyclo propyl)-1,2,4-oxadiazol-5 (4H)-one (1)

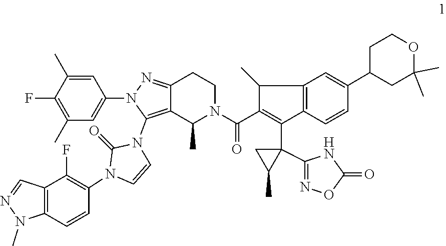 [SMILES: Cc1cc(-n2nc3c(c2-n2ccn(-c4ccc5c(cnn5C)c4F)c2=O)[C@H](C)N(C(=O)C2=C(C4(c5noc(=O)[nH]5)C[C@@H]4C)c4ccc(C5CCOC(C)(C)C5)cc4C2C)CC3)cc(C)c1F]

### Methyl 6-bromo-1-methyl-1H-indole-2-carboxylate (1a)

The title compound methyl 6-bromo-1-methyl-1H-indole-2-carboxylate (1a) was prepared according to the method described in WO2022147302.

### methyl 6-(2,2-dimethyltetrahydro-2H-pyran-4-yl)-1-methyl-1H-indole-2-carboxylate (1b)

To a mixture of Zn powder (678 mg, 10.41 mmol) in DMF (5 mL) was added TMSCl (1.0 mol/L in THF, 0.38 mL, 0.372 mmol) and 1,2-dibromoethane (70.0 mg, 0.372 mmol) at RT. After the mixture was stirred 10 at RT for min, a solution of 4-iodo-2,2-dimethyltetrahydro-2H-pyran (1.80 g, 7.45 mmol) in DMF (5 mL) was added and stirred at RT for 30 min. Then Pd (OAc) 2 (100 mg, 0.446 mmol) and AmPhos (240 mg, 0.894 mmol) followed by methyl 6-bromo-1-methyl-1H-indole-2-carboxylate (1a) (1.0 g, 3.72 mmol) was added at RT and the mixture was stirred at 50° C. for 2 h. The mixture was diluted with MTBE and H₂O, the organic layer was washed with brine, dried over Na₂SO₄, and concentrated. The residue was purified by column chromatography on silica gel eluting with PE/EtOAc (20:1~ 10:1) to give the title compound methyl 6-(2,2-dimethyltetrahydro-2H-pyran-4-yl)-1-methyl-1H-indole-2-carboxylate (1b). MS-ESI (m/z): 302 [M+1]⁺.

### methyl 3-bromo-6-(2,2-dimethyltetrahydro-2H-pyran-4-yl)-1-methyl-1H-indole-2-carboxylate (1c)

To a solution of methyl 6-(2,2-dimethyltetrahydro-2H-pyran-4-yl)-1-methyl-1H-indole-2-carboxylate (1b) (200 mg, 0.662 mmol) in DMF (5 mL) was added a suspension of NBS (118 mg, 0.662 mmol) in DMF (2 mL) dropwise. The mixture was stirred at RT for 10 min. The mixture was diluted with EtOAc and quenched with saturated Na₂SO₃ aqueous solution, the organic layer was washed with brine, dried over MgSO₄, and concentrated. The residue was purified by column chromatography on silica gel eluting with PE/EtOAc (20:1~ 3:1) to give the title compound methyl 3-bromo-6-(2,2-dimethyltetrahydro-2H-pyran-4-yl)-1-methyl-1H-indole-2-carboxylate (1c). MS-ESI (m/z): 380, 382 [M+1]⁺.

### methyl 3-(cyanomethyl)-6-(2,2-dimethyltetrahydro-2H-pyran-4-yl)-1-methyl-1H-indole-2-carboxylate (1d)

A mixture of methyl 3-bromo-6-(2,2-dimethyltetrahydro-2H-pyran-4-yl)-1-methyl-1H-indole-2-carboxylate (1c) (180 mg, 0.473 mmol), sodium 2-cyanoacetate (76 mg, 0.71 mmol), Pd₂(dba)₃ (30 mg, 0.032 mmol) and XantPhos (40 mg, 0.097 mmol) in diglyme (5 mL) was stirred at 140° C. for 40 min under microwave. The mixture was cooled to RT and diluted with water and EtOAc, the organic layer was washed with H₂O and brine, dried over Na₂SO₄, and concentrated. The residue was purified by column chromatography on silica gel eluting with PE/EtOAc (20:1~ 5:1) to give the title compound methyl 3-(cyanomethyl)-6-(2,2-dimethyltetrahydro-2H-pyran-4-yl)-1-methyl-1H-indole-2-carboxylate (1d). MS-ESI (m/z): 341 [M+1]⁺.

### 3-((2S)-1-cyano-2-methylcyclopropyl)-6-(2,2-dimethyltetrahydro-2H-pyran-4-yl)-1-methyl-1H-indole-2-carboxylic acid (1e)

To a solution of methyl 3-(cyanomethyl)-6-(2,2-dimethyltetrahydro-2H-pyran-4-yl)-1-methyl-1H-indole-2-carboxylate (1d) (140 mg, 0.411 mmol) and (R)-4-methyl-1,3,2-dioxathiolane 2,2-dioxide (56.8 mg, 0.411 mmol) in THF (8 mL) was added KHMDS (1.0 mol/L in THF, 2.05 mL, 2.05 mmol) under ice-bath. The reaction mixture stirred at 0° C. for 1 h. The mixture was quenched with sat. citric acid (PH=4~ 5) and extracted with EtOAc. The extracts were washed with water and brine, dried over Na₂SO₄ and concentrated. The residue was purified by PTLC eluting with DCM/MeOH (20:1) to give title compound 3-((2S)-1-cyano-2-methylcyclopropyl)-6-(2,2-dimethyltetrahydro-2H-pyran-4-yl)-1-methyl-1H-indole-2-carboxylic acid (1e). MS-ESI (m/z): 367 [M+1]⁺.

### (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one hydrochloride (1f)

The title compound(S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imid azol-2-one hydrochloride (1f) was prepared according to the method described in US2019225604A1.

### (2S)-1-(6-(2,2-dimethyltetrahydro-2H-pyran-4-yl)-2-(S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5, 6,7-tetrahydro-2H-pyrazolo[4,3-c]pyridine-5-carbonyl)-1-methyl-1H-indol-3-yl)-2-methylcyclopro pane-1-carbonitrile (1g)

A mixture of 3-((2S)-1-cyano-2-methylcyclopropyl)-6-(2,2-dimethyltetrahydro-2H-pyran-4-yl)-1-methyl-1H-indole-2-carboxylic acid (1e) (40 mg, 0.109 mmol), (S)-1-(4-fluoro-1-methyl-1H-indazol-5-yl)-3-(2-(4-fluoro-3,5-dimethylphenyl)-4-methyl-4,5,6,7-tetrahydro-2-pyrazolo[4,3-c]p yridin-3-yl)-1,3-dihydro-2H-imidazol-2-one hydrochloride (1f) (47 mg, 0.087 mmol), HATU (62 mg, 0.163 mmol), DIEA (42 mg, 0.327 mmol) and DMAP (6.6 mg, 0.054 mmol) in DMF (1 mL) was stirred at RT for overnight. The mixture was diluted with water and EtOAc, the organic layer was washed with H₂O and brine, dried over Na₂SO₄, and concentrated. The residue was purified by/ACN (150:40) PTLC eluting with DCM to give the title compound (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylph enyl)-4-methyl-4,5,6,7-tetrahydro-2H-pyrazolo[4,3-c]pyridine-5-carbonyl)-1-methyl-1H-indol-3-yl)-2-methylcyclopropane-1-carbonitrile (1g). MS-ESI (m/z): 838 [M+1]⁺.

### (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5 6,7-tetrahydro-2H-pyrazolo[4,3-c]pyridine-5-carbonyl)-1-methyl-1H-indol-3-yl)-N-hydroxy-2-met hylcyclopropane-1-carboximidamide (1h)

A mixture of (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1-methyl-1H-indol-3-yl)-2-meth ylcyclopropane-1-carbonitrile (1g) (30 mg, 0.00357 mmol), aq·NH₂OH (50% 180 mg, 2.72 mmol) in IPA (2 mL) was stirred at 85° C. for 4 h. The mixture was diluted with water and EtOAc, the organic layer was washed with H₂O and brine, dried over Na₂SO₄, and concentrated to give the crude product of (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-car bonyl)-1-methyl-1H-indol-3-yl)-N-hydroxy-2-methylcyclopropane-1-carboximidamide (1h), which was used for next step directly. MS-ESI (m/z): 871 [M+1]⁺.

### 3-(2S)-1-(6-(2,2-dimethyltetrahydro-2H-pyran-4-yl)-2-(S)-3-(3-(4-fluoro-1-methy l-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1-methyl-1H-indol-3-yl)-2-methylcyclo propyl)-1,2,4-oxadiazol-5 (4H)-one (1)

To a solution of (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1-methyl-1H-indol-3-yl)-N-hydr oxy-2-methylcyclopropane-1-carboximidamide (1h) (32 mg, 0.037 mmol) in DMSO (2.5 mL) was added DBU (14 mg, 0.092 mmol) and CDI (12 mg, 0.074 mmol). The mixture was stirred at RT for 1 h. The mixture was quenched with water and extracted with EtOAc. The extracts were washed with and brine, dried with Na₂SO₄ and concentrated. The residue was purified by PTLC eluting with DCM MeOH (50:1) to give the title compound 3-((2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetra hydro-2H-pyrazolo[4,3-c]pyridine-5-carbonyl)-1-methyl-1H-indol-3-yl)-2-methylcyclopropyl)-1,2, 4-oxadiazol-5 (4H)-one (1). MS-ESI (m/z): 897 [M+1]⁺.

### Examples 2

### 3-((1S,2S)-1-(5-(2,2-dimethyltetrahydro-2H-pyran-4-yl)-2-((S)-3-(1-(4-fluoro-1-me thyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopr opyl)-1,2,4-oxadiazol-5 (4H)-one (2)

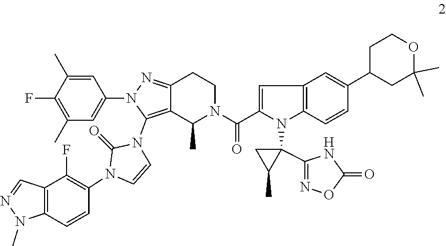 [SMILES: Cc1cc(-n2nc3c(c2-n2ccn(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F]

### Tert-butyl (S)-3-amino-2-(4-fluoro-3,5-dimethylphenyl)-4-methyl-2,4,6,7-tetrahydro-5H-pyrazolo[4,3-c]pyridine-5-carboxylate (2a)

The title compound tert-butyl (S)-3-amino-2-(4-fluoro-3,5-dimethylphenyl)-4-methyl-2,4,6,7-tetrahydro-5H-pyrazolo[4,3-c]pyridine-5-carboxylate (2a) was prepared according to the method described in US2019225604A1.

### Tert-butyl (S)-2-(4-fluoro-3,5-dimethylphenyl)-3-(hydrazinecarboxamido)-4-methyl-2,4,6,7-tetrahydro-5H-pyrazolo[4,3-c]pyridine-5-carboxylate (2b)

To a solution of triphosgene (720 mg, 2.42 mmol) in DCM (25 mL) was added tert-butyl (S)-3-amino-2-(4-fluoro-3,5-dimethylphenyl)-4-methyl-2,4,6,7-tetrahydro-5H-pyrazolo[4,3-c]pyridi ne-5-carboxylate (2a) (500 mg, 1.33 mmol) followed by TEA (2.5 mL, 17.8 mmol). The reaction mixture stirred at RT for 1 h and added to a solution of N₂H₄·H₂O (1.66 g, 26.56 mmol) in THF (10 mL). After stirring at RT for 5-10 min, the mixture was diluted with water and extracted with MTBE. The extracts were washed with water and brine, dried over Na₂SO₄ and concentrated. The residue was purified by recrystallized with PE/MTBE (10:1) to give title compound tert-butyl (S)-2-(4-fluoro-3,5-dimethylphenyl)-3-(hydrazinecarboxamido)-4-methyl-2,4,6,7-tetrahydro-5H-pyrazolo[4,3-c]pyridine-5-carboxylate (2b). MS-ESI (m/z): 433 [M+1]⁺.

### Tert-butyl (S)-2-(4-fluoro-3,5-dimethylphenyl)-4-methyl-3-(5-oxo-1,5-dihydro-4H-1,2,4-triazol-4-yl)-2,4,6,7-tetrahydro-5H-pyrazolo[4,3-c]pyridine-5-carboxylate (2c)

To a solution of tert-butyl (S)-2-(4-fluoro-3,5-dimethylphenyl)-3-(hydrazinecarboxamido)-4-methyl-2,4,6,7-tetrahydro-5H-pyrazolo[4,3-c]pyridine-5-carboxylate (2b) (467 mg, 1.079 mmol) in EtOH (10 mL) was added CH (OMe) 3 (911 mg, 8.58 mmol) and PTSA (10 mg, 0.052 mmol). The mixture was stirred at 80° C. for 2 h. The mixture was concentrated. The residue was purified by column chromatography on silica gel eluting with PE/EtOAc (10:1~ 2:1) to give the title compound tert-butyl (S)-2-(4-fluoro-3,5-dimethylphenyl)-4-methyl-3-(5-oxo-1,5-dihydro-4H-1,2,4-triazol-4-yl)-2,4,6,7-t etrahydro-5H-pyrazolo[4,3-c]pyridine-5-carboxylate (2c). MS-ESI (m/z): 443 [M+1]⁺.

### tert-butyl (S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-2,4,6,7-tetrahydro-5H-pyrazolo[4,3-c]pyridine-5-ca rboxylate (2d)

A mixture of tert-butyl (S)-2-(4-fluoro-3,5-dimethylphenyl)-4-methyl-3-(5-oxo-1,5-dihydro-4H-1,2,4-triazol-4-yl)-2,4,6,7-tetrahydro-5H-pyrazolo[4,3-c]pyridine-5-carboxylate (2c) (14 mg, 0.0316 mmol), 4-fluoro-1-methyl-5-(4,4,5,5-tetramethyl-1,3,2-dioxaborolan-2-yl)-1H-indazole (17.5 mg, 0.063 mmol), Cu (OAc) 2 (8.6 mg, 0.047 mmol), TEMPO (9.8 mg, 0.063 mmol), 4A MS (25 mg) and Py (5.0 mg, 0.063 mmol) in DMF (0.5 mL) was stirred at RT for 2.5 days. The mixture was diluted with EtOAc and water, filtered, the organic layer was washed with brine, dried over Na₂SO₄, and concentrated. The residue was purified by PTLC eluting with DCM/ACN (15:1) to give the title compound tert-butyl (S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-2,4,6,7-tetrahydro-5H-pyrazolo[4,3-c]pyridine-5-carboxylate (2d). MS-ESI (m/z): 591 [M+1]⁺.

### (S)-2-(4-fluoro-1-methyl-1H-indazol-5-yl)-4-(2-(4-fluoro-3,5-dimethylphenyl)-4-methyl-4,5,6,7-tetrahydro-2H-pyrazolo[4,3-c]pyridin-3-yl)-2,4-dihydro-3H-1,2,4-triazol-3-one (2e)

A mixture of tert-butyl (S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-2,4,6,7-tetrahydro-5H-pyrazolo[4, 3-c]pyridine-5-carboxylate (2d) (3.5 mg, 0.0059 mmol) and 4 N HCl in dioxane (0.5 mL) in DCM (0.5 mL) was stirred at RT for 0.5 h. The mixture was concentrated to give the crude product of (S)-2-(4-fluoro-1-methyl-1H-indazol-5-yl)-4-(2-(4-fluoro-3,5-dimethylphenyl)-4-methyl-4,5,6,7-tetrahydro-2H-pyrazolo[4,3-c]pyridin-3-yl)-2,4-dihydro-3H-1,2,4-triazol-3-one (2e), which was used for next step directly. MS-ESI (m/z): 491 [M+1]⁺.

### 3-((1S,2S)-1-(5-(2,2-dimethyltetrahydro-2H-pyran-4-yl)-2-((S)-3-(1-(4-fluoro-1-me thyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopr opyl)-1,2,4-oxadiazol-5 (4H)-one (2)

A mixture of(S)-2-(4-fluoro-1-methyl-1H-indazol-5-yl)-4-(2-(4-fluoro-3,5-dimethylphenyl)-4-methyl-4,5,6,7-tetrahydro-2H-pyrazolo[4,3-c]pyridin-3-yl)-2,4-dihydro-3H-1,2, 4-triazol-3-one (2e) (crude product), 5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (US2019225604A1) (3.0 mg, 0.0073 mmol), HATU (4.5 mg, 0.0118 mmol), DMAP (2.0 mg, 0.016 mmol) and DIPEA (5.0 mg, 0.0387 mmol) in DMF (0.5 mL) was stirred at RT for overnight. The mixture was diluted with water and extracted with EtOAc, the organic layer was washed with brine, dried over Na₂SO₄, and concentrated. The residue was purified by PTLC eluting with EtOAc/PE (65:35) to give the title compound 3-((1S,2S)-1-(5-(2,2-dimethyltetrahydro-2H-pyran-4-yl)-2-((S)-3-(1-(4-fluoro-1-methyl-1H-indazol-5-yl)-5-oxo-1,5-dihy dro-4H-1,2,4-triazol-4-yl)-2-(4-fluoro-3,5-dimethylphenyl)-4-methyl-4,5,6,7-tetrahydro-2H-pyrazol o[4,3-c]pyridine-5-carbonyl)-1H-indol-1-yl)-2-methylcyclopropyl)-1,2,4-oxadiazol-5 (4H)-one (2). MS-ESI (m/z): 884 [M+1]⁺.

### Examples 3

### 3-(2S)-1-(6-(2,2-dimethyltetrahydro-2H-pyran-4-yl)-2-(S)-3-(3-(4-fluoro-1-methy 1-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)-2-methylcyclopr opyl)-1,2,4-oxadiazol-5 (4H)-one (3)

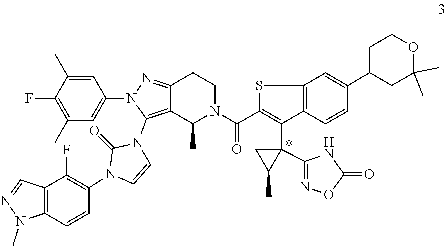 [SMILES: Cc1cc(-n2nc3c(c2-n2ccn(-c4ccc5c(cnn5C)c4F)c2=O)[C@H](C)N(C(=O)c2sc4cc(C5CCOC(C)(C)C5)ccc4c2C2(c4noc(=O)[nH]4)C[C@@H]2C)CC3)cc(C)c1F]

### Ethyl 6-bromo-3-methylbenzo[b]thiophene-2-carboxylate (3a)

To a solution of 1-(4-bromo-2-fluorophenyl) ethan-1-one (5.0 g, 23 mmol) and K₂CO₃ (4.3 g, 31.1 mmol) in DMF (30 mL) was added ethyl 2-mercaptoacetate (3.04 g, 25.3 mmol) at 0° C. After stirring at RT for 16 h, the mixture was warmed to 80° C. and stirred for 24 h. The mixture was filtered, the filtrate was washed with H₂O and extracted with EtOAc, dried over Na₂SO₄ and concentrated. The residue was purified by column chromatography on silica gel eluting with PE/EtOAc (20:1) to give the title compound ethyl 6-bromo-3-methylbenzo[b]thiophene-2-carboxylate (3a). MS-ESI (m/z): 299, 301 [M+1]⁺.

### Ethyl 6-bromo-3-(bromomethyl)benzo[b]thiophene-2-carboxylate (3b)

To a solution of ethyl 6-bromo-3-methylbenzo[b]thiophene-2-carboxylate (3a) (2.4 g, 8.02 mmol) in CCl₄ (72 mL) was added NBS (3.4 g, 19.1 mmol) and AIBN (0.23 g, 1.77 mmol) at RT. The mixture was stirred at 80° C. for 2 h. Then the mixture was cooled to RT and filtered. The filtered cake was washed with EtOAc, the filtrate was diluted with water, the organic layer was washed with brine, dried over Na₂SO₄, and concentrated. The residue was purified by column chromatography on silica gel eluting with EtOAc/PE (1:5) to give the title compound ethyl 6-bromo-3-(bromomethyl)benzo[b]thiophene-2-carboxylate (3b). MS-ESI (m/z): 297, 299 [M−Br+H]⁺.

### Ethyl 6-bromo-3-(cyanomethyl)benzo[b]thiophene-2-carboxylate (3c)

To a solution of ethyl 6-bromo-3-(bromomethyl)benzo[b]thiophene-2-carboxylate (3b) (3.0 g, 8.02 mmol) in MeCN (50 mL) was added TMSCN (1.03 g, 10.4 mmol) followed by TABF (1 M in THF, 10.4 mL, 10.4 mmol). The reaction mixture was stirred at RT for 1 h. The mixture was quenched with NaHCO₃ aqueous solution and extracted with EtOAc. The extracts were washed with brine, dried over Na₂SO₄ and concentrated. The residue was purified by column chromatography on silica gel eluting with PE/EtOAc (20:1~ 8:1) to give the title compound ethyl 6-bromo-3-(cyanomethyl)benzo[b]thiophene-2-carboxylate (3c). MS-ESI (m/z): 324, 326 [M+1]⁺.

### Ethyl 3-(cyanomethyl)-6-(2,2-dimethyltetrahydro-2H-pyran-4-yl)benzo[b]thiophene-2-carboxylate (3d)

To a mixture of Zn powder (264 mg, 4.07 mmol) in DMF (5 mL) was added TMSCl (1.0 mol/L in THF, 0.19 mL, 0.19 mmol) and 1,2-dibromoethane (34.8 mg, 0.185 mmol) at RT. After the mixture was stirred at RT for 10 min, a solution of 4-iodo-2,2-dimethyltetrahydro-2H-pyran (0.892 g, 3.70 mmol) in DMF (5 mL) was added and stirred at RT for 30 min. Then Pd (OAc) 2 (100 mg, 0.446 mmol) and AmPhos (240 mg, 0.894 mmol) followed by ethyl 6-bromo-3-(cyanomethyl)benzo[b]thiophene-2-carboxylate (3c) (0.6 g, 1.85 mmol) was added at RT and the mixture was stirred at 50° C. for 2 h. The mixture was diluted with MTBE and H₂O, the organic layer was washed with brine, dried over Na₂SO₄, and concentrated. The residue was purified by column chromatography on silica gel eluting with PE/EtOAc (20:1~ 10:1) to give the title compound ethyl 3-(cyanomethyl)-6-(2,2-dimethyltetrahydro-2H-pyran-4-yl)benzo[b]thiophene-2-carboxylate (3d). MS-ESI (m/z): 358 [M+1]⁺.

### 3-(Cyanomethyl)-6-(2,2-dimethyltetrahydro-2H-pyran-4-yl)benzo[b]thiophene-2-c arboxylic acid (3e)

A solution of ethyl 3-(cyanomethyl)-6-(2,2-dimethyltetrahydro-2H-pyran-4-yl)benzo[b]thiophene-2-carboxylate (3d) (320 mg, 0.893 mmol) and LiOH·H₂O (94 mg, 2.2 mmol) in MeOH/THF/H₂O (5/5/5 mL) was stirred at RT for 1 h. The mixture was diluted with water and acidified with citric acid to PH=5-6. The mixture was extracted with EtOAc, the extracts were washed with brine, dried over MgSO₄ and concentrated to give the crude product of 3-(cyanomethyl)-6-(2,2-dimethyltetrahydro-2H-pyran-4-yl)benzo[b]thiophene-2-carboxylic acid (3e), which was used for next step directly. MS-ESI (m/z): 330 [M+1]⁺.

### 2-(6-(2,2-Dimethyltetrahydro-2H-pyran-4-yl)-2-((S)-3-(3-(4-fluoro-1-methyl-1H-in dazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)acetonitrile (3f)

A mixture of 3-(cyanomethyl)-6-(2,2-dimethyltetrahydro-2H-pyran-4-yl)benzo[b]thiophene-2-carboxylic acid (3e) (37 mg, 0.113 mmol), (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one hydrochloride (1f) (59 mg, 0.113 mmol), HATU (64 mg, 0.169 mmol), DIEA (0.1 mL, 0.565 mmol) and DMAP (20 mg, 0.163 mmol) in DMF (1 mL) was stirred at RT for overnight. The mixture was diluted with water and EtOAc, the organic layer was washed with H₂O and brine, dried over Na₂SO₄, and concentrated. The residue was purified by PTLC eluting with PE/EtOAc (1:1) give the to title compound 2-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)acetonitrile (3f). MS-ESI (m/z): 801 [M+1]⁺.

### (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5 6,7-tetrahydro-2H-pyrazolo[4,3-c]pyridine-5-carbonyl)benzo[b]thiophen-3-yl)-2-methylcycloprop ane-1-carbonitrile (3g)

To a solution 2-(6-(2,2-dimethyltetrahydro-2H-pyran-4-yl)-2-((S)-3-(3-(4-fluoro-of 1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)acetonit rile (3f) (5.0 mg, 0.0062 mmol) and (R)-4-methyl-1,3,2-dioxathiolane 2,2-dioxide (0.86 mg, 0.0062 mmol) in THF (0.3 mL) was added KHMDS (1.0 mol/L in THF, 0.1 mL, 0.1 mmol) under ice-bath. The reaction mixture stirred at 0° C. for 1 h. The mixture was quenched with sat. citric acid (PH=4~ 5) and extracted with EtOAc. The extracts were washed with water and brine, dried over Na₂SO₄ and concentrated. The residue was purified by PTLC eluting with DCM/MeOH (20:1) to give title compound (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-car bonyl)benzo[b]thiophen-3-yl)-2-methylcyclopropane-1-carbonitrile (3g). MS-ESI (m/z): 841 [M+1]⁺.

### 3-((2S)-1-(6-(2,2-dimethyltetrahydro-2H-pyran-4-yl)-2-((S)-3-(3-(4-fluoro-1-methy 1-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)-2-methylcyclopr opyl)-1,2,4-oxadiazol-5 (4H)-one (3)

The title compound 3-((2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylph enyl)-4-methyl-4,5,6,7-tetrahydro-2H-pyrazolo[4,3-c]pyridine-5-carbonyl)benzo[b]thiophen-3-yl)-2-methylcyclopropyl)-1,2,4-oxadiazol-5 (4H)-one (3) was prepared according to the synthetic method of 1 by replacing (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1-methyl-1H-indol-3-yl)-2-methylcyclopropane-1-carbonit rile (1g) with (2S)-1-(6-(2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzo[b]thiophen-3-yl)-2-methylcyclopropane-1-carbonitril e (3g). MS-ESI (m/z): 900 [M+1]⁺.

### Examples 4

3-((2S)-1-(6-(2,2-dimethyltetrahydro-2H-pyran-4-yl)-2-((S)-3-(3-(4-fluoro-1-methy 1-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)benzofuran-3-yl)-2-methylcyclopropyl)-1,2,4-oxadiazol-5 (4H)-one (4)

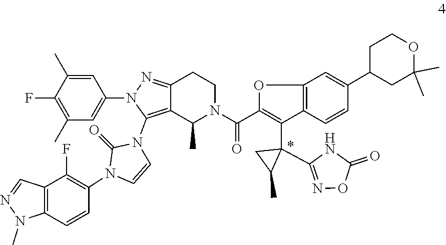 [SMILES: Cc1cc(-n2nc3c(c2-n2ccn(-c4ccc5c(cnn5C)c4F)c2=O)[C@H](C)N(C(=O)c2oc4cc(C5CCOC(C)(C)C5)ccc4c2C2(c4noc(=O)[nH]4)C[C@@H]2C)CC3)cc(C)c1F]

The title compound 3-((2S)-1-(6-(2,2-dimethyltetrahydro-2H-pyran-4-yl)-2-((S)-3-(3-(4-compound fluoro-1-methyl-1H-indazol-5-yl)-2-oxo-2,3-dihydro-1H-imidazol-1-yl)-2-(4-fluoro-3,5-dimethylph enyl)-4-methyl-4,5,6,7-tetrahydro-2H-pyrazolo[4,3-c]pyridine-5-carbonyl)benzofuran-3-yl)-2-meth ylcyclopropyl)-1,2,4-oxadiazol-5 (4H)-one (4) was prepared according to the synthetic method of 3 by replacing 1-(4-bromo-2-fluorophenyl) ethan-1-one and ethyl 2-mercaptoacetate with 1-(4-bromo-2-hydroxyphenyl) ethan-1-one and ethyl 2-bromoacetate. MS-ESI (m/z): 884 [M+1]⁺.

Following essentially the same procedures described for Examples 1-4, Examples 5-63 listed in Table 1 were/can be prepared from the appropriate starting materials which are commercially available or known in the literature. The structures and names of Examples 5-63 are given in Table 1.

TABLE 1

| EX- | | | |
|---|---|---|---|
| AM- | | | |
| PLE | Structure | Name | DATA* |
| 5 | 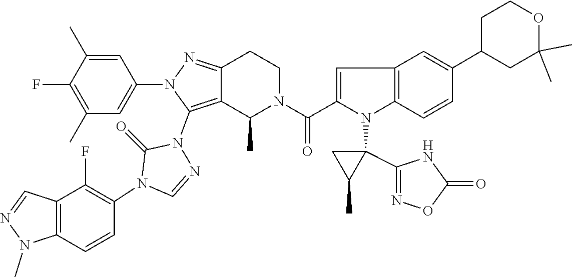 [SMILES: Cc1cc(-n2nc3c(c2-n2ncn(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2- dimethyltetrahydro- 2H-pyran-4-yl)- 2-((S)-3-(4-(4-fluoro- 1-methyl-1H-indazol- 5-yl)-5-oxo-4,5-dihydro- 1H-1,2,4-triazol-1-yl)-2- (4-fluoro-3,5-dimethyl- phenyl)-4-methyl-4,5,6,7- tetrahydro-2H-pyrazolo [4,3-c]pyridine-5- carbonyl)-1H-indol- 1-yl)-2-methyl- cyclopropyl)-1,2,4- oxadiazol-5(4H)-one | // |
| 6 | 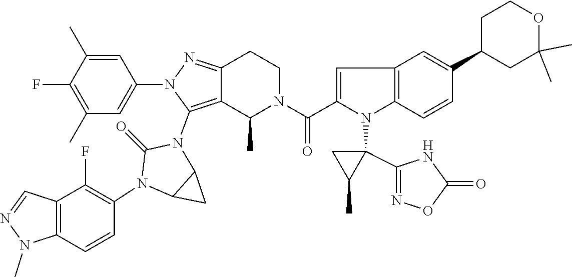 [SMILES: Cc1cc(-n2nc3c(c2N2C(=O)N(c4ccc5c(cnn5C)c4F)C4CC42)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((4S)-3-(4- (4-fluoro-1-methyl-1H- indazol-5-yl)-3-oxo-2,4- diazabicyclo[3.1.0]hexan- 2-yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)- 2-methylcyclopropyl)- 1,2,4-oxadiazol-5(4H)- one | // |
| 7 | 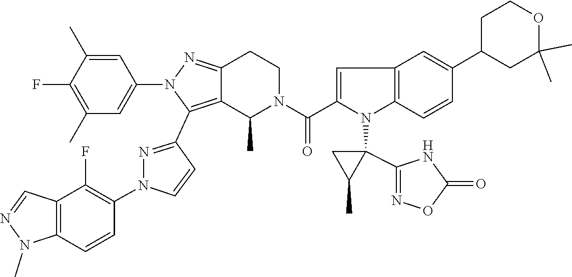 [SMILES: Cc1cc(-n2nc3c(c2-c2ccn(-c4ccc5c(cnn5C)c4F)n2)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5- (2,2-dimethyl- tetrahydro-2H-pyran-4- yl)-2-((S)-3-(1-(4-fluoro- 1-methyl-1H-indazol- 5-yl)-1H-pyrazol-3-yl)- 2-(4-fluoro-3,5-dimethyl- phenyl)-4-methyl-4,5,6,7- tetrahydro-2H-pyrazolo [4,3-c]pyridine- 5-carbonyl)- 1H-indol-1-yl)-2-methyl- cyclopropyl)-1,2,4- oxadiazol-5(4H)-one | // |
| 8 | 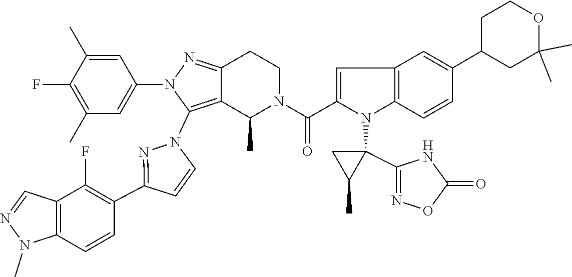 [SMILES: Cc1cc(-n2nc3c(c2-n2ccc(-c4ccc5c(cnn5C)c4F)n2)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2- dimethyltetrahydro- 2H-pyran-4-yl)- 2-((S)-3-(3-(4-fluoro- 1-methyl-1H-indazol- 5-yl)-1H-pyrazol-1-yl)-2- (4-fluoro-3,5-dimethyl- phenyl)-4-methyl-4,5,6,7- tetrahydro-2H-pyrazolo [4,3-c]pyridine- 5-carbonyl)- 1H-indol-1-yl)-2- methylcyclopropyl)- 1,2,4-oxadiazol-5(4H)- one | // |
| 9 | 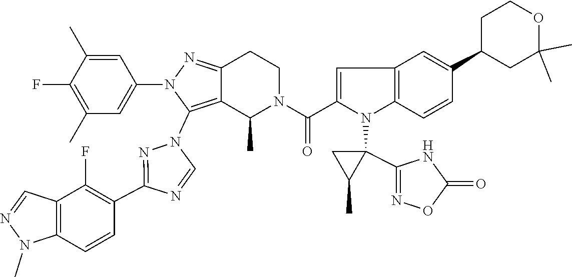 [SMILES: Cc1cc(-n2nc3c(c2-n2cnc(-c4ccc5c(cnn5C)c4F)n2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((S)-3-(3- (4-fluoro-1-methyl-1H- indazol-5-yl)-1H-1,2,4- triazol-1-yl)-2-(4- fluoro-3,5-dimethyl- phenyl)-4-methyl- 4,5,6,7-tetrahydro-2H- pyrazolo[4,3-c]pyridine- 5-carbonyl)-1H-indol- 1-yl)-2- methylcyclopropyl)- 1,2,4-oxadiazol-5(4H)- one | // |
| 10 | 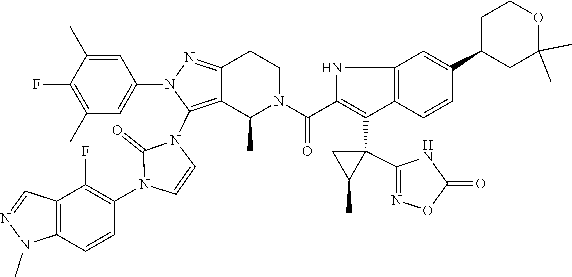 [SMILES: Cc1cc(-n2nc3c(c2-n2ccn(-c4ccc5c(cnn5C)c4F)c2=O)[C@H](C)N(C(=O)c2[nH]c4cc([C@H]5CCOC(C)(C)C5)ccc4c2[C@@]2(c4noc(=O)[nH]4)C[C@@H]2C)CC3)cc(C)c1F] | 3-((1R,2S)-1-(6-((S)-2,2- dimethyltetrahydro-2H- pyran-4-yl)-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H-pyrazolo [4,3-c]pyridine-5- carbonyl)-1H-indol- 3-yl)-2-methyl- cyclopropyl)-1,2,4- oxadiazol-5(4H)-one | // |
| 11 | 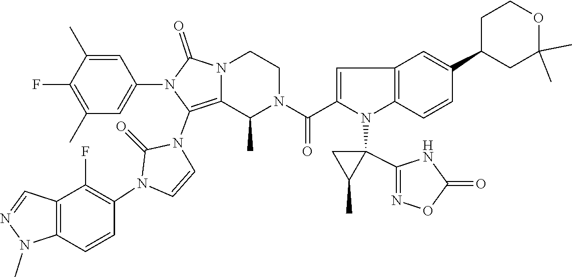 [SMILES: Cc1cc(-n2c(-n3ccn(-c4ccc5c(cnn5C)c4F)c3=O)c3n(c2=O)CCN(C(=O)c2cc4cc([C@H]5CCOC(C)(C)C5)ccc4n2[C@@]2(c4noc(=O)[nH]4)C[C@@H]2C)[C@H]3C)cc(C)c1F] | 3-((1S,2S)-1-(5-((S)-2,2- dimethyltetrahydro-2H- pyran-4-yl)-2-((S)-1-(3- (4-fluoro-1-methyl-1H- indazol-5-yl)-2-oxo-2,3- dihydro-1H- imidazol-1-yl)- 2-(4-fluoro-3,5-dimethyl- phenyl)-8-methyl-3-oxo- 2,3,5,6,7,8- hexahydroimidazo [1,5-a]pyrazine-7- carbonyl)-1H-indol-1-yl)- 2-methylcyclopropyl)- 1,2,4-oxadiazol-5(4H)- one | // |
| 12 | 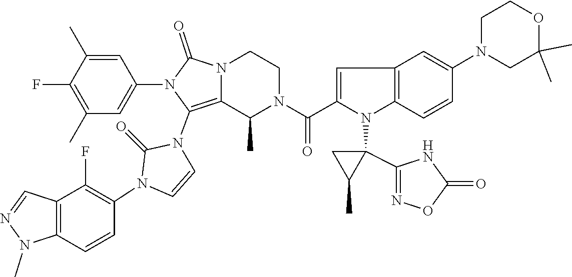 [SMILES: Cc1cc(-n2c(-n3ccn(-c4ccc5c(cnn5C)c4F)c3=O)c3n(c2=O)CCN(C(=O)c2cc4cc(N5CCOC(C)(C)C5)ccc4n2[C@@]2(c4noc(=O)[nH]4)C[C@@H]2C)[C@H]3C)cc(C)c1F] | 3-((1S,2S)-1-(5- (2,2-dimethyl- morpholino)-2-((S)- 1-(3-(4-fluoro-1-methyl- 1H-indazol-5-yl)- 2-oxo-2,3-dihydro- 1H-imidazol-1-yl)- 2-(4-fluoro-3,5-dimethyl- phenyl)-8-methyl-3-oxo- 2,3,5,6,7,8-hexahydro- imidazo[1,5-a]pyrazine- 7-carbonyl)-1H-indol-1- yl)-2-methylcyclopropyl)- 1,2,4-oxadiazol-5(4H)- one | // |
| 13 | 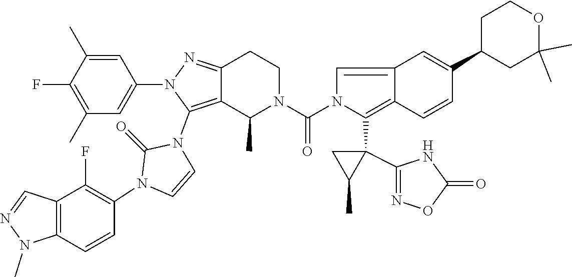 [SMILES: Cc1cc(-n2nc3c(c2-n2ccn(-c4ccc5c(cnn5C)c4F)c2=O)[C@H](C)N(C(=O)n2cc4cc([C@H]5CCOC(C)(C)C5)ccc4c2[C@@]2(c4noc(=O)[nH]4)C[C@@H]2C)CC3)cc(C)c1F] | 3-((1R,2S)-1-(5-((S)-2,2- dimethyltetrahydro-2H- pyran-4-yl)-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H-pyrazolo [4,3-c]pyridine-5- carbonyl)-2H- isoindol-1-yl)-2- methylcyclopropyl)-1,2,4- oxadiazol-5(4H)-one | // |
| 14 | 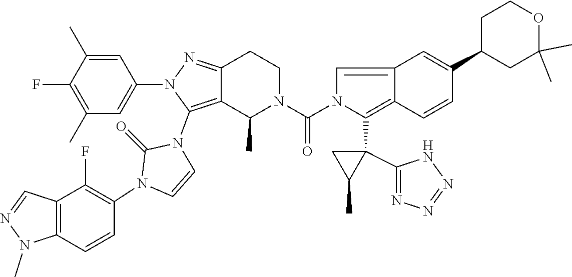 [SMILES: Cc1cc(-n2nc3c(c2-n2ccn(-c4ccc5c(cnn5C)c4F)c2=O)[C@H](C)N(C(=O)n2cc4cc([C@H]5CCOC(C)(C)C5)ccc4c2[C@@]2(c4nnn[nH]4)C[C@@H]2C)CC3)cc(C)c1F] | 1-((S)-5-(5-((S)- 2,2-dimethyl- tetrahydro-2H-pyran-4- yl)-1-((1R,2S)-2-methyl- 1-(1H-tetrazol-5-yl)cyclo- propyl)-2H-isoindole-2- carbonyl)-2-(4-fluoro-3,5- dimethylphenyl)-4-methyl- 4,5,6,7-tetrahydro-2H- pyrazolo[4,3-c]pyridin-3- yl)-3-(4-fluoro-1-methyl- 1H-indazol-5-yl)-1,3- dihydro-2H-imidazol-2- one | // |
| 15 | 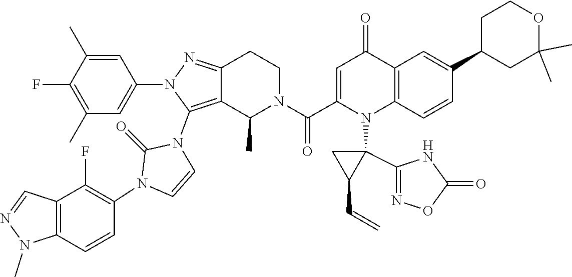 [SMILES: C=C[C@H]1C[C@]1(c1noc(=O)[nH]1)n1c(C(=O)N2CCc3nn(-c4cc(C)c(F)c(C)c4)c(-n4ccn(-c5ccc6c(cnn6C)c5F)c4=O)c3[C@@H]2C)cc(=O)c2cc([C@H]3CCOC(C)(C)C3)ccc21] | 3-((1S,2R)-1-(6-((S)-2,2- dimethyltetrahydro-2H- pyran-4-yl)-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H- pyrazolo[4,3-c] pyridine-5-carbonyl)- 4-oxoquinolin-1(4H)-yl)- 2-vinylcyclopropyl)-1,2,4- oxadiazol-5(4H)-one | // |
| 16 | 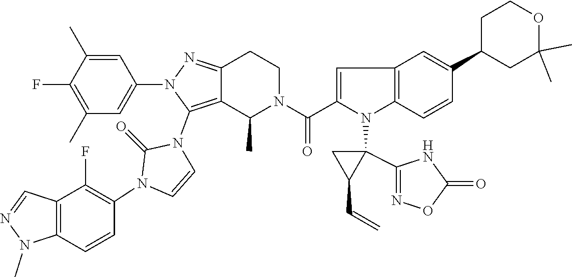 [SMILES: C=C[C@H]1C[C@]1(c1noc(=O)[nH]1)n1c(C(=O)N2CCc3nn(-c4cc(C)c(F)c(C)c4)c(-n4ccn(-c5ccc6c(cnn6C)c5F)c4=O)c3[C@@H]2C)cc2cc([C@H]3CCOC(C)(C)C3)ccc21] | 3-((1S,2R)-1-(5-((S)-2,2- dimethyltetrahydro-2H- pyran-4-yl)-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H- pyrazolo[4,3-c] pyridine-5-carbonyl)- 1H-indol-1-yl)-2-vinyl- cyclopropyl)-1,2,4- oxadiazol-5(4H)-one | // |
| 17 | 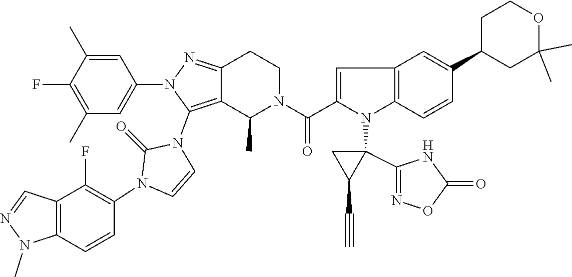 [SMILES: C#C[C@H]1C[C@]1(c1noc(=O)[nH]1)n1c(C(=O)N2CCc3nn(-c4cc(C)c(F)c(C)c4)c(-n4ccn(-c5ccc6c(cnn6C)c5F)c4=O)c3[C@@H]2C)cc2cc([C@H]3CCOC(C)(C)C3)ccc21] | 3-((1S,2R)-1-(5-((S)-2,2- dimethyltetrahydro-2H- pyran-4-yl)-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H- pyrazolo[4,3-c] pyridine-5-carbonyl)- 1H-indol-1-yl)-2-ethynyl- cyclopropyl)-1,2,4- oxadiazol-5(4H)-one | // |
| 18 | 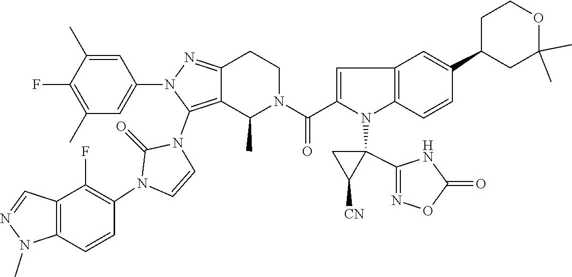 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C#N)CC3)cc(C)c1F] | (1S,2S)-2-(5-((S)-2,2- dimethyltetrahydro- 2H-pyran-4-yl)- 2-((S)-3-(3-(4-fluoro- 1-methyl-1H-indazol- 5-yl)-2-oxo-2,3-dihydro- 1H-imidazol-1-yl)-2-(4- fluoro-3,5- dimethylphenyl)- 4-methyl-4,5,6,7-tetra- hydro-2H-pyrazolo[4,3- c]pyridine-5- carbonyl)-1H- indol-1-yl)-2-(5-oxo-4,5- dihydro-1,2,4-oxadiazol- 3-yl)cyclopropane-1- carbonitrile | // |
| 19 | 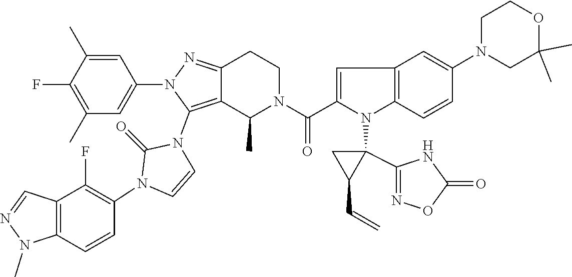 [SMILES: C=C[C@H]1C[C@]1(c1noc(=O)[nH]1)n1c(C(=O)N2CCc3nn(-c4cc(C)c(F)c(C)c4)c(-n4ccn(-c5ccc6c(cnn6C)c5F)c4=O)c3[C@@H]2C)cc2cc(N3CCOC(C)(C)C3)ccc21] | 3-((1S,2R)-1-(5- (2,2-dimethyl- morpholino)-2-((S)- 3-(3-(4-fluoro-1-methyl- 1H-indazol-5-yl)-2- oxo-2,3-dihydro- 1H-imidazol-1-yl)- 2-(4-fluoro-3,5-dimethyl- phenyl)-4-methyl-4,5,6,7- tetrahydro-2H-pyrazolo [4,3-c]pyridine-5- carbonyl)- 1H-indol-1-yl)-2- vinylcyclopropyl)-1,2,4- oxadiazol-5(4H)-one | // |
| 20 | 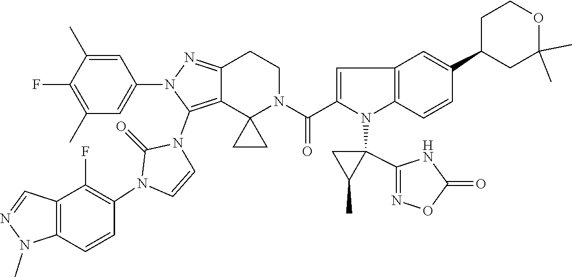 [SMILES: Cc1cc(-n2nc3c(c2-n2ccn(-c4ccc5c(cnn5C)c4F)c2=O)C2(CC2)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-(3′-(3-(4- fluoro-1-methyl- 1H-indazol- 5-yl)-2-oxo-2,3-dihydro- 1H-imidazol-1-yl)-2′-(4- fluoro-3,5- dimethylphenyl)- 2′,5′,6′,7′-tetrahydrospiro [cyclopropane-1,4′- pyrazolo[4,3-c]pyridine]- 5′-carbonyl)- 1H-indol-1-yl)- 2-methylcyclopropyl)- 1,2,4-oxadiazol-5(4H)- one | // |
| 21 | 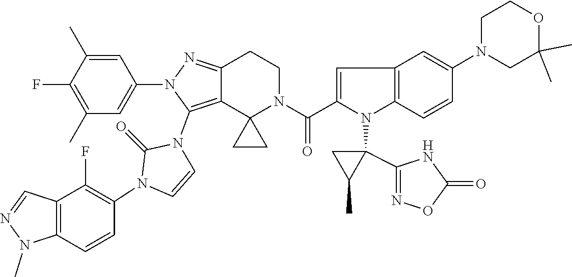 [SMILES: Cc1cc(-n2nc3c(c2-n2ccn(-c4ccc5c(cnn5C)c4F)c2=O)C2(CC2)N(C(=O)c2cc4cc(N5CCOC(C)(C)C5)ccc4n2[C@@]2(c4noc(=O)[nH]4)C[C@@H]2C)CC3)cc(C)c1F] | 3-((1S,2S)-1-(5- (2,2-dimethyl- morpholino)-2-(3′-(3- (4-fluoro-1-methyl-1H- indazol-5-yl)-2-oxo-2,3- dihydro-1H- imidazol-1-yl)- 2′-(4-fluoro-3,5-dimethyl- phenyl)-2′,5′,6′,7′- tetrahydrospiro [cyclopropane-1,4′- pyrazolo[4,3-c]pyridine]- 5′-carbonyl)-1H-indol- 1-yl)-2-methyl- cyclopropyl)- 1,2,4-oxadiazol-5(4H)- one | // |
| 22 | 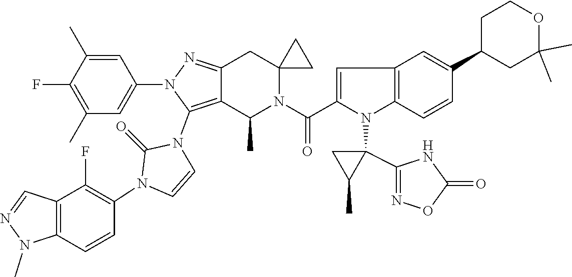 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)C2(CC2)C3)cc(C)c1F] | 3-((1S,2S)-1-(5-((S)-2,2- dimethyltetrahydro-2H- pyran-4-yl)-2-((S)-3′-(3- (4-fluoro-1-methyl-1H- indazol-5-yl)-2-oxo-2,3- dihydro-1H- imidazol-1-yl)- 2′-(4-fluoro-3,5-dimethyl- phenyl)-4′-methyl- 2′,4′,5′,7′-tetrahydrospiro [cyclopropane- 1,6′-pyrazolo[4,3- c]pyridine]-5′-carbonyl)- 1H-indol-1-yl)-2-methyl cyclopropyl)-1,2,4- oxadiazol-5(4H)-one | // |
| 23 | 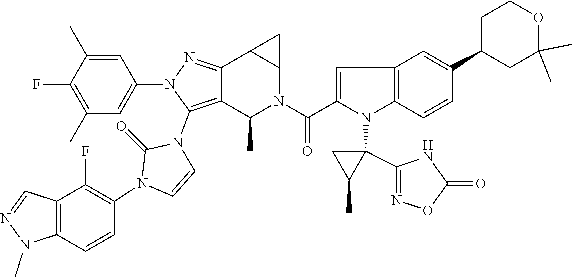 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)C2CC32)cc(C)c1F] | 3-((1S,2S)-1-(5-((S)-2,2- dimethyltetrahydro-2H- pyran-4-yl)-2-((4S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl- 2,4,5,5a,6,6a-hexahydro- cyclopropa[b] pyrazolo[3,4- d]pyridine-5-carbonyl)- 1H-indol-1-yl)-2- methylcyclopropyl)-1,2,4- oxadiazol-5(4H)-one | // |
| 24 | 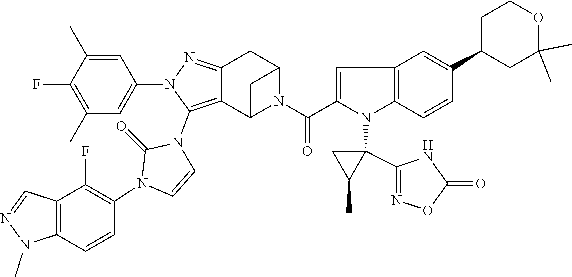 [SMILES: Cc1cc(-n2nc3c(c2-n2ccn(-c4ccc5c(cnn5C)c4F)c2=O)C2CC(C3)N2C(=O)c2cc3cc([C@H]4CCOC(C)(C)C4)ccc3n2[C@@]2(c3noc(=O)[nH]3)C[C@@H]2C)cc(C)c1F] | 3-((1S,2S)-1-(5-((S)-2,2- dimethyltetrahydro-2H- pyran-4-yl)-2-(3-(3-(4- fluoro-1-methyl- 1H-indazol- 5-yl)-2-oxo-2,3-dihydro- 1H-imidazol-1-yl)-2-(4- fluoro-3,5- dimethylphenyl)- 4,5,6,7-tetrahydro-2H- 4,6-epiminoindazole-8- carbonyl)-1H-indol-1- yl)-2-methyl- cyclopropyl)-1,2,4- oxadiazol-5(4H)-one | // |
| 25 | 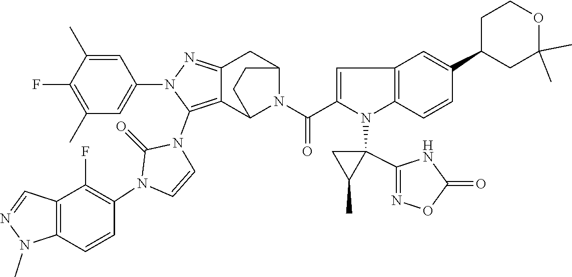 [SMILES: Cc1cc(-n2nc3c(c2-n2ccn(-c4ccc5c(cnn5C)c4F)c2=O)C2CCC(C3)N2C(=O)c2cc3cc([C@H]4CCOC(C)(C)C4)ccc3n2[C@@]2(c3noc(=O)[nH]3)C[C@@H]2C)cc(C)c1F] | 3-((1S,2S)-1-(5-((S)-2,2- dimethyltetrahydro-2H- pyran-4-yl)-2-(3-(3-(4- fluoro-1-methyl- 1H-indazol- 5-yl)-2-oxo-2,3-dihydro- 1H-imidazol-1-yl)-2-(4- fluoro-3,5- dimethylphenyl)- 2,4,5,6,7,8-hexahydro- 4,7-epiminocyclohepta [c]pyrazole-9-carbonyl)- 1H-indol-1-yl)-2-methyl- cyclopropyl)-1,2,4- oxadiazol-5(4H)-one | // |
| 26 | 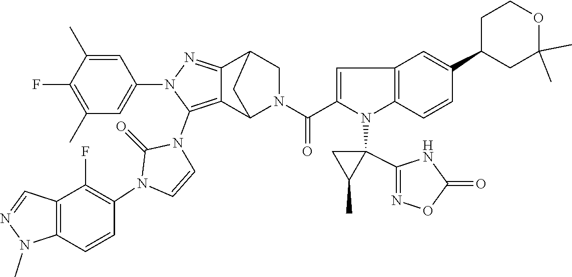 [SMILES: Cc1cc(-n2nc3c(c2-n2ccn(-c4ccc5c(cnn5C)c4F)c2=O)C2CC3CN2C(=O)c2cc3cc([C@H]4CCOC(C)(C)C4)ccc3n2[C@@]2(c3noc(=O)[nH]3)C[C@@H]2C)cc(C)c1F] | 3-((1S,2S)-1-(5-((S)-2,2- dimethyltetrahydro-2H- pyran-4-yl)-2-(3-(3-(4- fluoro-1-methyl- 1H-indazol- 5-yl)-2-oxo-2,3-dihydro- 1H-imidazol-1-yl)-2-(4- fluoro-3,5- dimethylphenyl)- 4,5,6,7-tetrahydro-2H- 4,7-methanopyrazolo[4,3- c]pyridine-5-carbonyl)- 1H-indol-1-yl)-2-methyl- cyclopropyl)-1,2,4- oxadiazol-5(4H)-one | // |
| 27 | 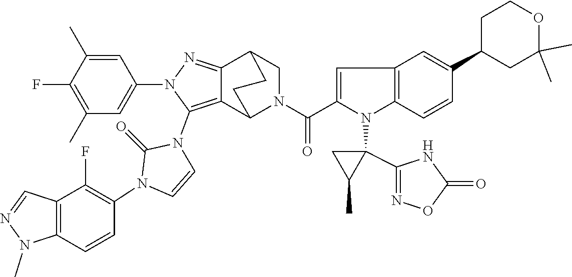 [SMILES: Cc1cc(-n2nc3c(c2-n2ccn(-c4ccc5c(cnn5C)c4F)c2=O)C2CCC3CN2C(=O)c2cc3cc([C@H]4CCOC(C)(C)C4)ccc3n2[C@@]2(c3noc(=O)[nH]3)C[C@@H]2C)cc(C)c1F] | 3-((1S,2S)-1-(5-((S)-2,2- dimethyltetrahydro-2H- pyran-4-yl)-2-(3-(3-(4- fluoro-1-methyl- 1H-indazol- 5-yl)-2-oxo-2,3-dihydro- 1H-imidazol-1-yl)-2-(4- fluoro-3,5- dimethylphenyl)- 4,5,6,7-tetrahydro-2H- 4,7-(epiminomethano) indazole-9-carbonyl)-1H- indol-1-yl)-2-methylcyclo- propyl)-1,2,4-oxadiazol- 5(4H)-one | // |
| 28 | 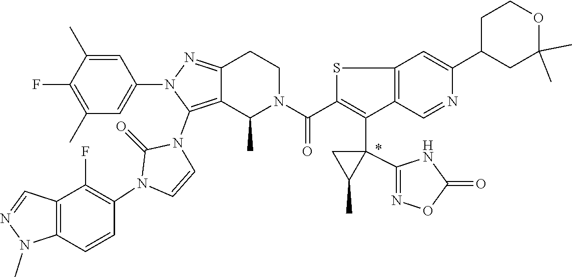 [SMILES: Cc1cc(-n2nc3c(c2-n2ccn(-c4ccc5c(cnn5C)c4F)c2=O)[C@H](C)N(C(=O)c2sc4cc(C5CCOC(C)(C)C5)ncc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-fluoro- 1-methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo[4,3-c] pyridine-5- carbonyl)thieno[3,2- c]pyridin-3-yl)-2- methylcyclopropyl)-1,2,4- oxadiazol-5(4H)-one | // |
| 29 | 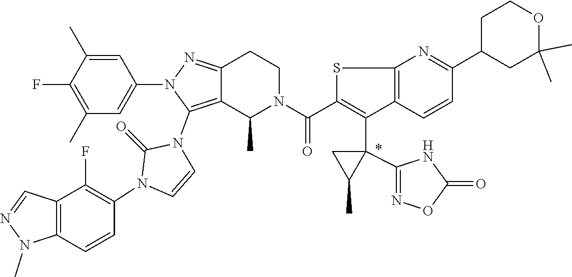 [SMILES: Cc1cc(-n2nc3c(c2-n2ccn(-c4ccc5c(cnn5C)c4F)c2=O)[C@H](C)N(C(=O)c2sc4nc(C5CCOC(C)(C)C5)ccc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-fluoro- 1-methyl-1H-indazol-5- yl)-2-oxo-2,3-dihydro- 1H-imidazol-1-yl)-2-(4- fluoro-3,5-dimethyl- phenyl)-4-methyl- 4,5,6,7-tetrahydro- 2H-pyrazolo[4,3-c] pyridine-5- carbonyl)thieno [2,3-b]pyridin-3-yl)-2- methylcyclopropyl)-1,2,4- oxadiazol-5(4H)-one | // |
| 30 | 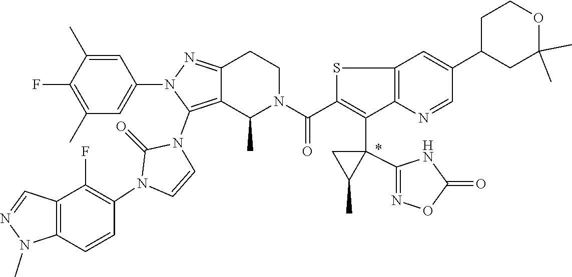 [SMILES: Cc1cc(-n2nc3c(c2-n2ccn(-c4ccc5c(cnn5C)c4F)c2=O)[C@H](C)N(C(=O)c2sc4cc(C5CCOC(C)(C)C5)cnc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3- (3-(4-fluoro-1- methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo[4,3- c]pyridine- 5-carbonyl)thieno[3,2- b]pyridin-3-yl)-2-methyl- cyclopropyl)-1,2,4- oxadiazol-5(4H)-one | // |
| 31 | 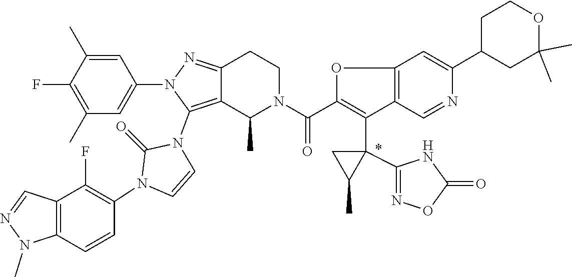 [SMILES: Cc1cc(-n2nc3c(c2-n2ccn(-c4ccc5c(cnn5C)c4F)c2=O)[C@H](C)N(C(=O)c2oc4cc(C5CCOC(C)(C)C5)ncc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-fluoro- 1-methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo[4,3- c]pyridine- 5-carbonyl)furo[3,2- c]pyridin-3-yl)-2-methyl- cyclopropyl)-1,2,4- oxadiazol-5(4H)-one | // |
| 32 | 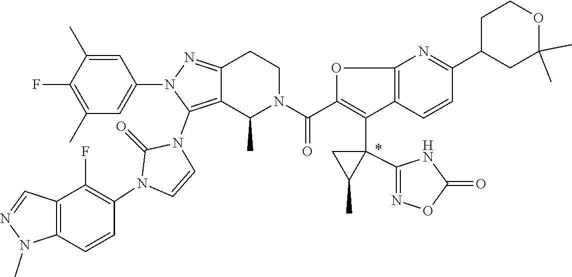 [SMILES: Cc1cc(-n2nc3c(c2-n2ccn(-c4ccc5c(cnn5C)c4F)c2=O)[C@H](C)N(C(=O)c2oc4nc(C5CCOC(C)(C)C5)ccc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-fluoro- 1-methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo[4,3- c]pyridine- 5-carbonyl)furo[2,3- b]pyridin-3-yl)-2-methyl- cyclopropyl)-1,2,4- oxadiazol-5(4H)-one | // |
| 33 | 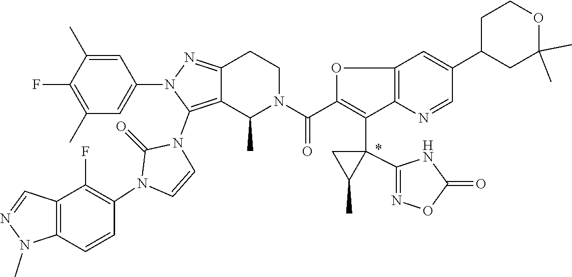 [SMILES: Cc1cc(-n2nc3c(c2-n2ccn(-c4ccc5c(cnn5C)c4F)c2=O)[C@H](C)N(C(=O)c2oc4cc(C5CCOC(C)(C)C5)cnc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-fluoro- 1-methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo[4,3- c]pyridine- 5-carbonyl)furo[3,2- b]pyridin-3-yl)-2-methyl- cyclopropyl)-1,2,4- oxadiazol-5(4H)-one | // |
| 34 | 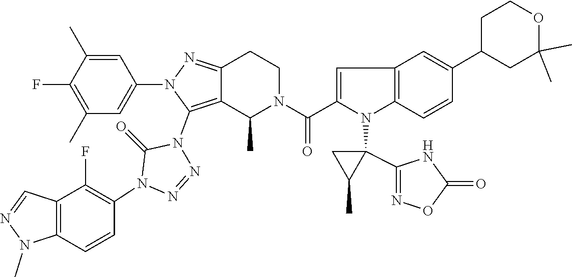 [SMILES: Cc1cc(-n2nc3c(c2-n2nnn(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2-di- methyltetrahydro-2H- pyran-4-yl)-2-((S)-3-(4- (4-fluoro-1-methyl- 1H-indazol- 5-yl)-5-oxo-4,5-dihydro- 1H-tetrazol-1-yl)-2-(4- fluoro-3,5- dimethylphenyl)- 4-methyl-4,5,6,7- tetrahydro-2H-pyrazolo [4,3-c]pyridine- 5-carbonyl)- 1H-indol-1-yl)-2-methyl- cyclopropyl)-1,2,4- oxadiazol-5(4H)-one | // |
| 35 | 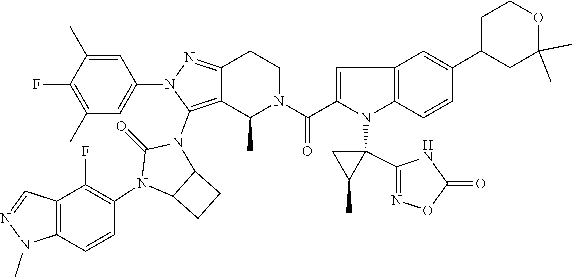 [SMILES: Cc1cc(-n2nc3c(c2N2C(=O)N(c4ccc5c(cnn5C)c4F)C4CCC42)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2-di- methyltetrahydro- 2H-pyran-4-yl)-2- ((4S)-3-(4-(4-fluoro- 1-methyl-1H-indazol- 5-yl)-3-oxo-2,4- diazabicyclo [3.2.0]heptan-2-yl)- 2-(4-fluoro-3,5-dimethyl- phenyl)-4-methyl-4,5,6,7- tetrahydro-2H-pyrazolo [4,3-c]pyridine- 5-carbonyl)- 1H-indol-1-yl)-2-methyl- cyclopropyl)-1,2,4- oxadiazol-5(4H)-one | // |
| 36 | 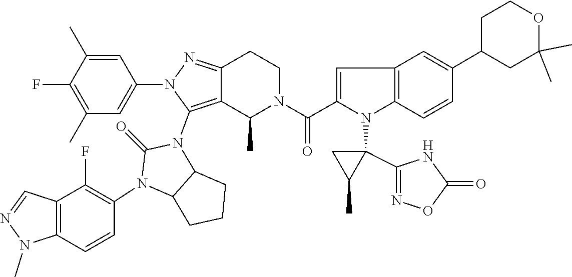 [SMILES: Cc1cc(-n2nc3c(c2N2C(=O)N(c4ccc5c(cnn5C)c4F)C4CCCC42)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2-di- methyltetrahydro- 2H-pyran-4-yl)-2- ((4S)-3-(3-(4-fluoro- 1-methyl-1H-indazol- 5-yl)-2-oxohexahydro- cyclopenta[d] imidazol-1(2H)- yl)-2-(4-fluoro-3,5- dimethylphenyl)-4- methyl-4,5,6,7- tetrahydro-2H-pyrazolo [4,3-c]pyridine-5- carbonyl)-1H-indol-1-yl)- 2-methylcyclopropyl)- 1,2,4-oxadiazol-5(4H)- one | // |
| 37 | 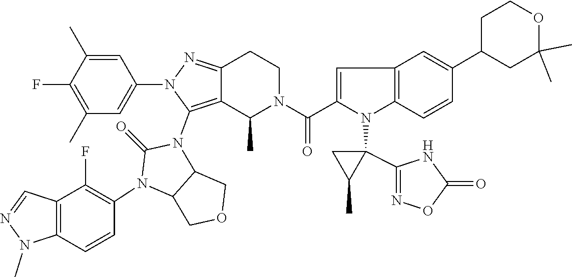 [SMILES: Cc1cc(-n2nc3c(c2N2C(=O)N(c4ccc5c(cnn5C)c4F)C4COCC42)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 1-((4S)-5-(5- (2,2-dimethyl- tetrahydro-2H-pyran-4- yl)-1-((1S,2S)-2-methyl- 1-(5-oxo-4,5-dihydro- 1,2,4-oxadiazol-3-yl) cyclopropyl)-1H- indole-2-carbonyl)- 2-(4-fluoro-3,5- dimethylphenyl)-4- methyl-4,5,6,7-tetrahydro- 2H-pyrazolo[4,3-c] pyridin-3-yl)-3-(4-fluoro- 1-methyl-1H-indazol-5- yl)tetrahydro-1H-furo [3,4-d]imidazol-2(3H)- one | // |
| 38 | 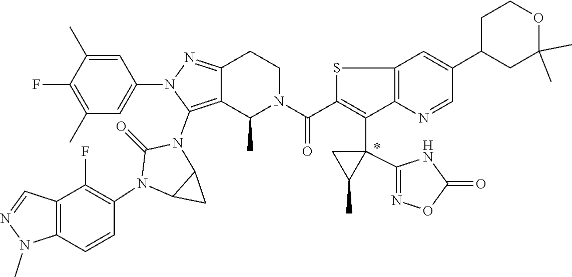 [SMILES: Cc1cc(-n2nc3c(c2N2C(=O)N(c4ccc5c(cnn5C)c4F)C4CC42)[C@H](C)N(C(=O)c2sc4cc(C5CCOC(C)(C)C5)cnc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((4S)-3-(4-(4-fluoro- 1-methyl-1H-indazol-5- yl)-3-oxo-2,4-diazabicyclo [3.1.0]hexan-2-yl)-2-(4- fluoro-3,5- dimethylphenyl)- 4-methyl-4,5,6,7- tetrahydro-2H-pyrazolo [4,3-c]pyridine- 5-carbonyl)thieno [3,2-b]pyridin-3-yl)- 2-methylcyclopropyl)- 1,2,4-oxadiazol- 5(4H)-one | // |
| 39 | 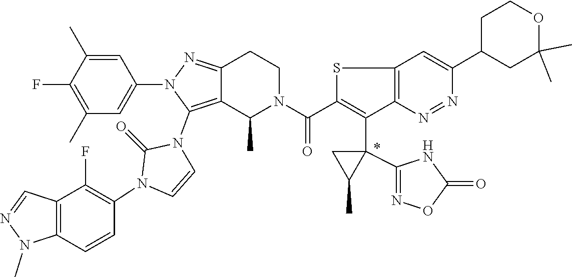 [SMILES: Cc1cc(-n2nc3c(c2-n2ccn(-c4ccc5c(cnn5C)c4F)c2=O)[C@H](C)N(C(=O)c2sc4cc(C5CCOC(C)(C)C5)nnc4c2C2(c4noc(=O)[nH]4)C[C@@H]2C)CC3)cc(C)c1F] | 3-((2S)-1-(3- (2,2-dimethyl- tetrahydro-2H-pyran-4- yl)-6-((S)-3-(3-(4-fluoro- 1-methyl-1H- indazol-5-yl)- 2-oxo-2,3-dihydro-1H- imidazol-1-yl)-2-(4- fluoro-3,5- dimethylphenyl)-4- methyl-4,5,6,7-tetrahydro- 2H-pyrazolo [4,3-c]pyridine- 5-carbonyl)thieno[3,2- c]pyridazin-7-yl)-2- methylcyclopropyl)-1,2,4- oxadiazol-5(4H)-one | // |
| 40 | 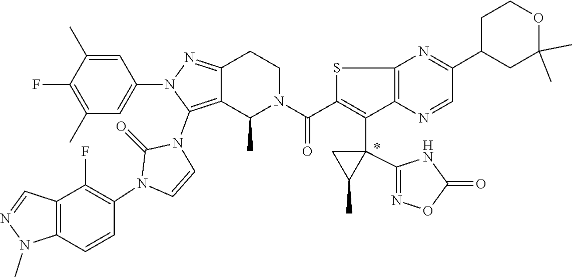 [SMILES: Cc1cc(-n2nc3c(c2-n2ccn(-c4ccc5c(cnn5C)c4F)c2=O)[C@H](C)N(C(=O)c2sc4nc(C5CCOC(C)(C)C5)cnc4c2C2(c4noc(=O)[nH]4)C[C@@H]2C)CC3)cc(C)c1F] | 3-((2S)-1-(3- (2,2-dimethyl- tetrahydro-2H-pyran-4- yl)-6-((S)-3-(3-(4-fluoro- 1-methyl-1H-indazol-5- yl)-2-oxo-2,3-dihydro- 1H-imidazol-1- yl)-2-(4-fluoro- 3,5-dimethylphenyl)-4- methyl-4,5,6,7-tetrahydro- 2H-pyrazolo [4,3-c]pyridine- 5-carbonyl)thieno[2,3- b]pyrazin-7-yl)-2-methyl- cyclopropyl)-1,2,4- oxadiazol-5(4H)-one | // |
| 41 | 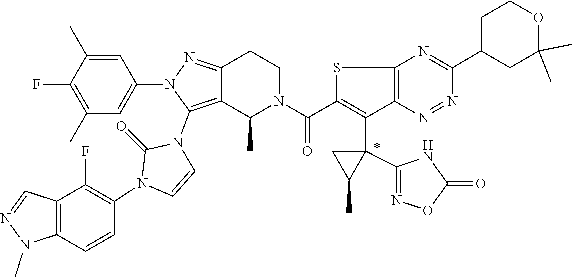 [SMILES: Cc1cc(-n2nc3c(c2-n2ccn(-c4ccc5c(cnn5C)c4F)c2=O)[C@H](C)N(C(=O)c2sc4nc(C5CCOC(C)(C)C5)nnc4c2C2(c4noc(=O)[nH]4)C[C@@H]2C)CC3)cc(C)c1F] | 3-((2S)-1-(3- (2,2-dimethyl- tetrahydro-2H-pyran-4- yl)-6-((S)-3-(3-(4-fluoro- 1-methyl-1H- indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo [4,3-c]pyridine- 5-carbonyl)thieno[2,3- e][1,2,4]triazin-7-yl)-2- methylcyclopropyl)-1,2,4- oxadiazol-5(4H)-one | // |
| 42 | 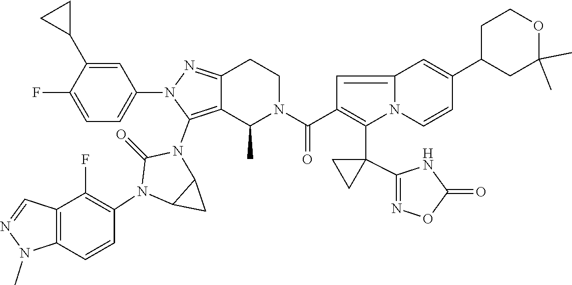 [SMILES: C[C@H]1c2c(nn(-c3ccc(F)c(C4CC4)c3)c2N2C(=O)N(c3ccc4c(cnn4C)c3F)C3CC32)CCN1C(=O)c1cc2cc(C3CCOC(C)(C)C3)ccn2c1C1(c2noc(=O)[nH]2)CC1] | 3-(1-(2-((4S)-2-(3-cyclo- propyl-4-fluorophenyl)-3- (4-(4-fluoro-1-methyl-1H- indazol-5-yl)-3-oxo-2,4- diazabicyclo[3.1.0]hexan- 2-yl)-4-methyl-4,5,6,7- tetrahydro-2H-pyrazolo [4,3-c]pyridine- 5-carbonyl)- 7-(2,2-dimethyltetrahydro- 2H-pyran-4-yl)indolizin- 3-yl)cyclopropyl)-1,2,4- oxadiazol-5(4H)-one | // |
| 43 | 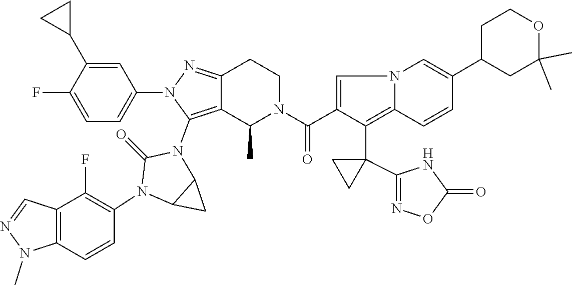 [SMILES: C[C@H]1c2c(nn(-c3ccc(F)c(C4CC4)c3)c2N2C(=O)N(c3ccc4c(cnn4C)c3F)C3CC32)CCN1C(=O)c1cn2cc(C3CCOC(C)(C)C3)ccc2c1C1(c2noc(=O)[nH]2)CC1] | 3-(1-(2-((4S)-2-(3-cyclo- propyl-4-fluorophenyl)-3- (4-(4-fluoro-1-methyl-1H- indazol-5-yl)-3-oxo-2,4- diazabicyclo[3.1.0]hexan- 2-yl)-4-methyl-4,5,6,7- tetrahydro-2H- pyrazolo[4,3- c]pyridine-5-carbonyl)- 6-(2,2-dimethyltetrahydro- 2H-pyran-4-yl)indolizin- 1-yl)cyclopropyl)-1,2,4- oxadiazol-5(4H)-one | // |
| 44 | 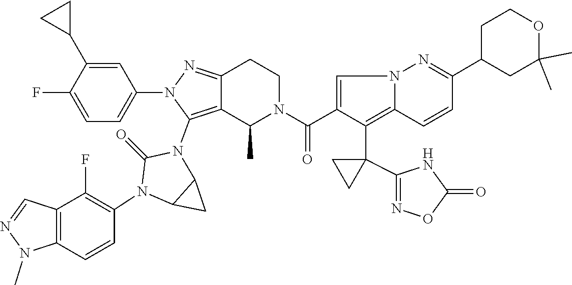 [SMILES: C[C@H]1c2c(nn(-c3ccc(F)c(C4CC4)c3)c2N2C(=O)N(c3ccc4c(cnn4C)c3F)C3CC32)CCN1C(=O)c1cn2nc(C3CCOC(C)(C)C3)ccc2c1C1(c2noc(=O)[nH]2)CC1] | 3-(1-(6-((4S)-2-(3-cyclo- propyl-4-fluorophenyl)-3- (4-(4-fluoro-1-methyl-1H- indazol-5-yl)-3-oxo-2,4- diazabicyclo[3.1.0]hexan- 2-yl)-4-methyl-4,5,6,7- tetrahydro-2H-pyrazolo [4,3-c]pyridine- 5-carbonyl)- 2-(2,2-dimethyltetrahydro- 2H-pyran-4-yl)pyrrolo [1,2-b]pyridazin-5-yl) cyclopropyl)-1,2,4- oxadiazol-5(4H)-one | // |
| 45 | 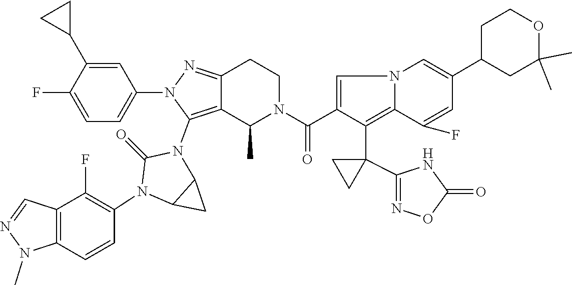 [SMILES: C[C@H]1c2c(nn(-c3ccc(F)c(C4CC4)c3)c2N2C(=O)N(c3ccc4c(cnn4C)c3F)C3CC32)CCN1C(=O)c1cn2cc(C3CCOC(C)(C)C3)cc(F)c2c1C1(c2noc(=O)[nH]2)CC1] | 3-(1-(2-((4S)-2-(3-cyclo- propyl-4-fluorophenyl)-3- (4-(4-fluoro-1-methyl-1H- indazol-5-yl)-3-oxo-2,4- diazabicyclo[3.1.0]hexan- 2-yl)-4-methyl-4,5,6,7- tetrahydro-2H- pyrazolo[4,3- c]pyridine-5-carbonyl)- 6-(2,2-dimethyltetrahydro- 2H-pyran-4-yl)-8- fluoroindolizin-1-yl) cyclopropyl)-1,2,4- oxadiazol-5(4H)-one | // |
| 46 | 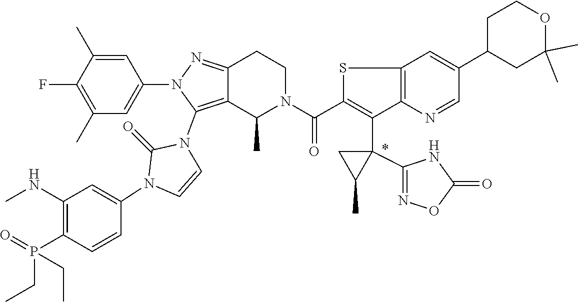 | 3-((2S)-1-(2-((S)-3-(3-(4- (diethylphosphoryl)-3- (methylamino)phenyl)-2- oxo-2,3-dihydro-1H- imidazol-1-yl)- 2-(4-fluoro-3,5- dimethylphenyl)-4-methyl- 4,5,6,7-tetrahydro-2H- pyrazolo[4,3-c]pyridine- 5-carbonyl)-6- (2,2-dimethyl- tetrahydro-2H-pyran- 4-yl)thieno[3,2-b]pyridin- 3-yl)-2-methylcyclo- propyl)-1,2,4-oxadiazol- 5(4H)-one | // |
| 47 | 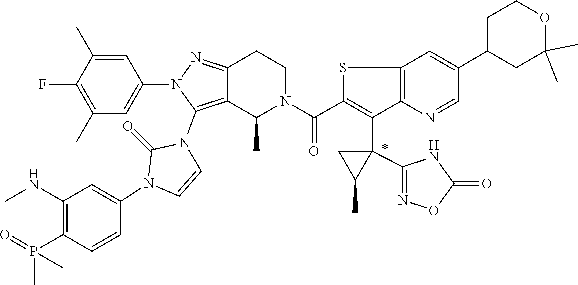 | 3-((2S)-1-(2-((S)-3-(3- (4-(dimethylphosphoryl)- 3-(methylamino)phenyl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3-c] pyridine-5-carbonyl)-6- (2,2-dimethyl- tetrahydro-2H- pyran-4-yl)thieno[3,2- b]pyridin-3-yl)-2-methyl- cyclopropyl)-1,2,4- oxadiazol-5(4H)-one | // |
| 48 | 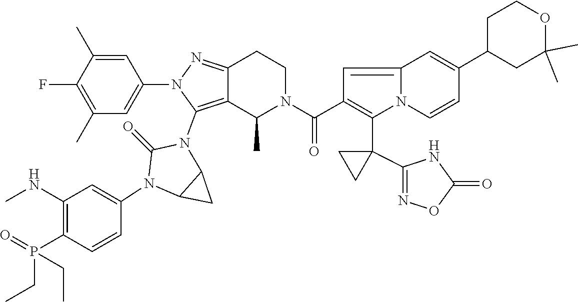 | 3-(1-(2-((4S)-3-(4-(4- (diethylphosphoryl)-3- (methylamino)phenyl)- 3-oxo-2,4-diazabicyclo [3.1.0]hexan-2-yl)-2-(4- fluoro-3,5- dimethylphenyl)- 4-methyl-4,5,6,7- tetrahydro- 2H-pyrazolo[4,3-c] pyridine-5-carbonyl)-7- (2,2-dimethyl- tetrahydro-2H- pyran-4-yl)indolizin-3- yl)cyclopropyl)-1,2,4- oxadiazol-5(4H)-one | // |
| 49 | 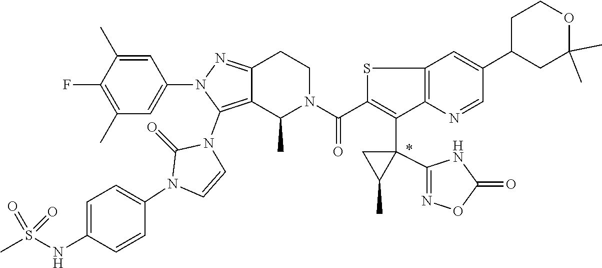 | N-(4-(3-((4S)-5-(6-(2,2- dimethyltetrahydro-2H- pyran-4-yl)-3-((2S)-2- methyl-1-(5- oxo-4,5-dihydro-1,2,4- oxadiazol-3-yl)cyclo- propyl)thieno[3,2-b] pyridine-2-carbonyl)-2- (4-fluoro-3,5-dimethyl- phenyl)-4-methyl-4,5,6,7- tetrahydro-2H- pyrazolo[4,3-c] pyridin-3-yl)-2-oxo-2,3- dihydro-1H-imidazol-1- yl)phenyl) methanesulfonamide | // |
| 50 | 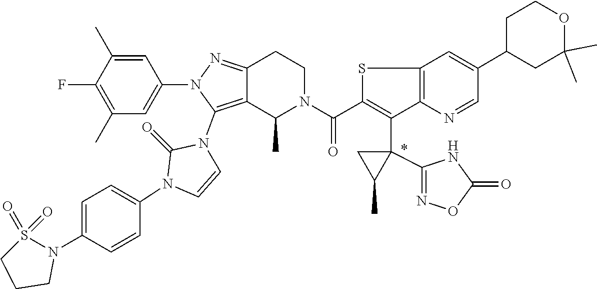 [SMILES: Cc1cc(-n2nc3c(c2-n2ccn(-c4ccc(N5CCCS5(=O)=O)cc4)c2=O)[C@H](C)N(C(=O)c2sc4cc(C5CCOC(C)(C)C5)cnc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((S)-3-(3-(4-(1,1- dioxidoisothiazolidin- 2-yl)phenyl)- 2-oxo-2,3-dihydro- 1H-imidazol-1-yl)-2- (4-fluoro-3,5-dimethyl- phenyl)-4-methyl-4,5,6,7- tetrahydro-2H-pyrazolo [4,3-c]pyridine- 5-carbonyl)thieno [3,2-b]pyridin-3-yl)- 2-methylcyclopropyl)- 1,2,4-oxadiazol- 5(4H)-one | // |
| 51 | 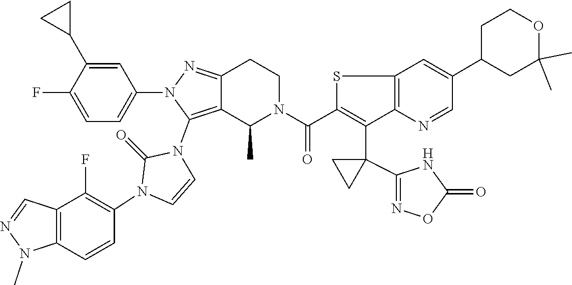 [SMILES: C[C@H]1c2c(nn(-c3ccc(F)c(C4CC4)c3)c2-n2ccn(-c3ccc4c(cnn4C)c3F)c2=O)CCN1C(=O)c1sc2cc(C3CCOC(C)(C)C3)cnc2c1C1(c2noc(=O)[nH]2)CC1] | 3-(1-(2-((S)-2-(3-cyclo- propyl-4-fluorophenyl)-3- (3-(4-fluoro-1-methyl- 1H-indazol-5-yl)- 2-oxo-2,3-dihydro- 1H-imidazol-1- yl)-4-methyl-4,5,6,7- tetrahydro-2H-pyrazolo [4,3-c]pyridine- 5-carbonyl)-6-(2,2- dimethyltetrahydro- 2H-pyran-4-yl)thieno [3,2-b]pyridin-3-yl)cyclo- propyl)-1,2,4-oxadiazol- 5(4H)-one | // |
| 52 | 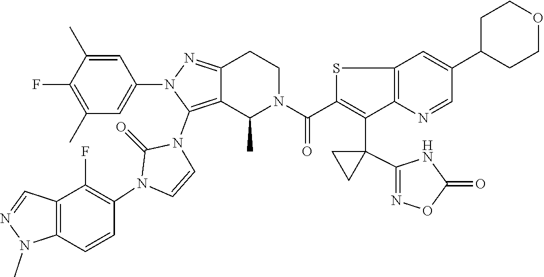 [SMILES: Cc1cc(-n2nc3c(c2-n2ccn(-c4ccc5c(cnn5C)c4F)c2=O)[C@H](C)N(C(=O)c2sc4cc(C5CCOCC5)cnc4c2C2(c4noc(=O)[nH]4)CC2)CC3)cc(C)c1F] | (S)-3-(1-(2-(3-(3-(4- fluoro-1-methyl- 1H-indazol-5-yl)- 2-oxo-2,3-dihydro- 1H-imidazol-1-yl)-2-(4- fluoro-3,5- dimethylphenyl)- 4-methyl-4,5,6,7- tetrahydro-2H-pyrazolo [4,3-c]pyridine- 5-carbonyl)- 6-(tetrahydro-2H-pyran- 4-yl)thieno[3,2-b]pyridin- 3-yl)cyclopropyl)-1,2,4- oxadiazol-5(4H)-one | // |
| 53 | 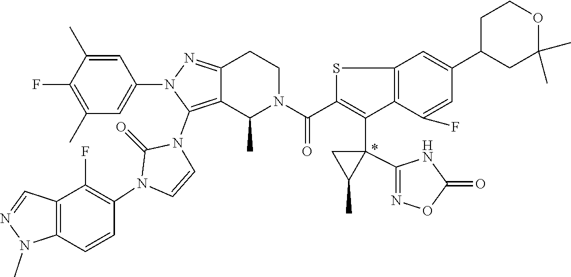 [SMILES: Cc1cc(-n2nc3c(c2-n2ccn(-c4ccc5c(cnn5C)c4F)c2=O)[C@H](C)N(C(=O)c2sc4cc(C5CCOC(C)(C)C5)cc(F)c4c2C2(c4noc(=O)[nH]4)C[C@@H]2C)CC3)cc(C)c1F] | 3-((2S)-1-(6- (2,2-dimethyl- tetrahydro-2H-pyran-4- yl)-4-fluoro-2-((S)-3-(3- (4-fluoro-1-methyl-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H-pyrazolo [4,3-c]pyridine- 5-carbonyl) benzo[b]thiophen-3-yl)- 2-methylcyclopropyl)- 1,2,4-oxadiazol-5(4H)- one | // |
| 54 | 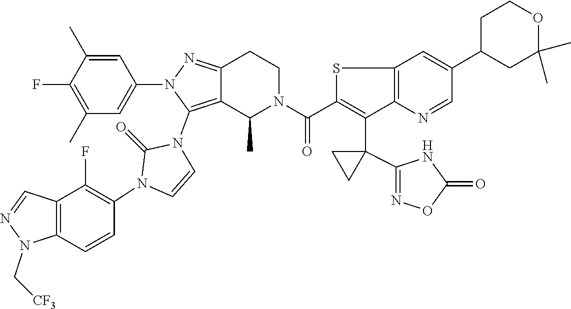 [SMILES: Cc1cc(-n2nc3c(c2-n2ccn(-c4ccc5c(cnn5CC(F)(F)F)c4F)c2=O)[C@H](C)N(C(=O)c2sc4cc(C5CCOC(C)(C)C5)cnc4c2C2(c4noc(=O)[nH]4)CC2)CC3)cc(C)c1F] | 3-(1-(6-(2,2-dimethyl- tetrahydro-2H- pyran-4-yl)- 2-((S)-3-(3-(4-fluoro-1- (2,2,2-trifluoroethyl)-1H- indazol-5-yl)-2-oxo-2,3- dihydro-1H- imidazol-1-yl)- 2-(4-fluoro-3,5-dimethyl- phenyl)-4-methyl-4,5,6,7- tetrahydro-2H-pyrazolo [4,3-c]pyridine- 5-carbonyl) thieno[3,2-b]pyridin-3- yl)cyclopropyl)-1,2,4- oxadiazol-5(4H)-one | // |
| 55 | 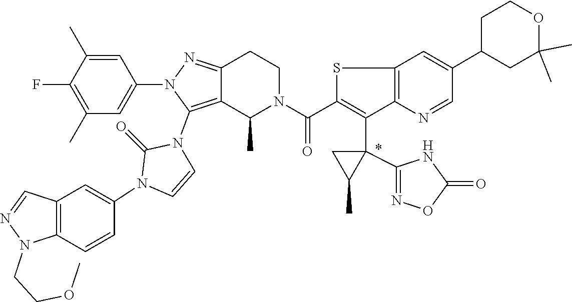 [SMILES: COCCn1ncc2cc(-n3ccn(-c4c5c(nn4-c4cc(C)c(F)c(C)c4)CCN(C(=O)c4sc6cc(C7CCOC(C)(C)C7)cnc6c4C4(c6noc(=O)[nH]6)C[C@@H]4C)[C@H]5C)c3=O)ccc21] | 3-((2S)-1-(6- (2,2-dimethyl- tetrahydro-2H-pyran-4- yl)-2-((S)-2-(4-fluoro-3,5- dimethylphenyl)-3-(3- (1-(2-methoxyethyl)-1H- indazol-5-yl)-2-oxo-2,3- dihydro-1H-imidazol-1- yl)-4-methyl-4,5,6,7- tetrahydro-2H-pyrazolo [4,3-c]pyridine- 5-carbonyl) thieno[3,2-b]pyridin-3- yl)-2-methylcyclopropyl)- 1,2,4-oxadiazol-5(4H)- one | // |
| 56 | 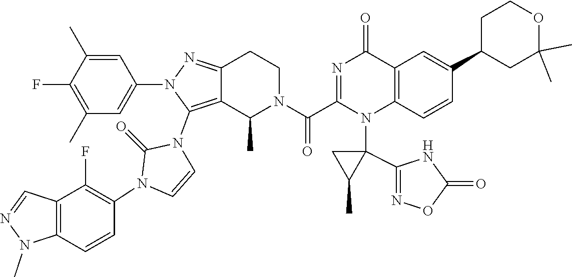 [SMILES: Cc1cc(-n2nc3c(c2-n2ccn(-c4ccc5c(cnn5C)c4F)c2=O)[C@H](C)N(C(=O)c2nc(=O)c4cc([C@H]5CCOC(C)(C)C5)ccc4n2C2(c4noc(=O)[nH]4)C[C@@H]2C)CC3)cc(C)c1F] | 3-((2S)-1-(6-((S)-2,2-di- methyltetrahydro- 2H-pyran-4-yl)- 2-((S)-3-(3-(4-fluoro- 1-methyl-1H-indazol- 5-yl)-2-oxo-2,3-dihydro- 1H-imidazol-1-yl)-2-(4- fluoro-3,5- dimethylphenyl)- 4-methyl-4,5,6,7- tetrahydro-2H-pyrazolo [4,3-c]pyridine- 5-carbonyl)-4- oxoquinazolin-1(4H)-yl)- 2-methylcyclopropyl)- 1,2,4-oxadiazol-5(4H)- one | // |
| 57 | 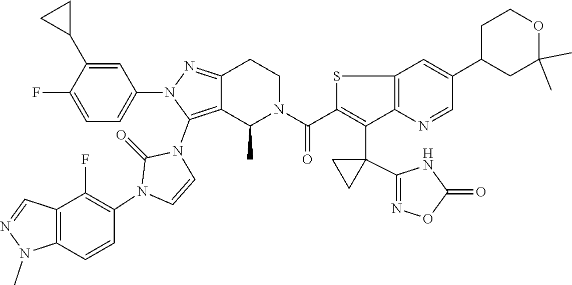 [SMILES: C[C@H]1c2c(nn(-c3ccc(F)c(C4CC4)c3)c2-n2ccn(-c3ccc4c(cnn4C)c3F)c2=O)CCN1C(=O)c1sc2cc(C3CCOC(C)(C)C3)cnc2c1C1(c2noc(=O)[nH]2)CC1] | 3-(1-(2-((S)-2-(3-cyclo- propyl-4-fluorophenyl)-3- (3-(4-fluoro-1-methyl- 1H-indazol-5-yl)-2- oxo-2,3-dihydro- 1H-imidazol-1- yl)-4-methyl-4,5,6,7- tetrahydro-2H- pyrazolo[4,3- c]pyridine-5-carbonyl)- 6-(2,2-dimethyltetrahydro- 2H-pyran-4-yl)thieno [3,2-b]pyridin-3-yl)cyclo- propyl)-1,2,4-oxadiazol- 5(4H)-one | // |
| 58 | 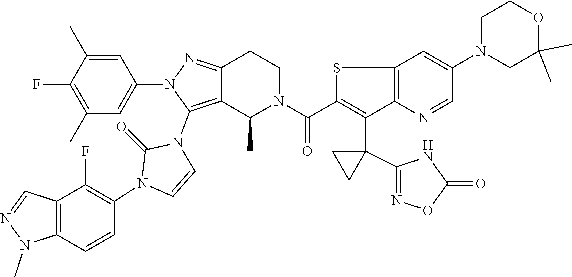 [SMILES: Cc1cc(-n2nc3c(c2-n2ccn(-c4ccc5c(cnn5C)c4F)c2=O)[C@H](C)N(C(=O)c2sc4cc(N5CCOC(C)(C)C5)cnc4c2C2(c4noc(=O)[nH]4)CC2)CC3)cc(C)c1F] | (S)-3-(1-(6-(2,2-dimethyl- morpholino)-2-(3-(3-(4- fluoro-1-methyl-1H- indazol-5-yl)-2-oxo-2,3- dihydro-1H-imidazol-1- yl)-2-(4-fluoro-3,5- dimethylphenyl)-4- methyl-4,5,6,7- tetrahydro-2H- pyrazolo[4,3- c]pyridine-5- carbonyl)thieno[3,2-b] pyridin-3-yl)cyclopropyl)- 1,2,4-oxadiazol-5(4H)-one | // |
| 59 | 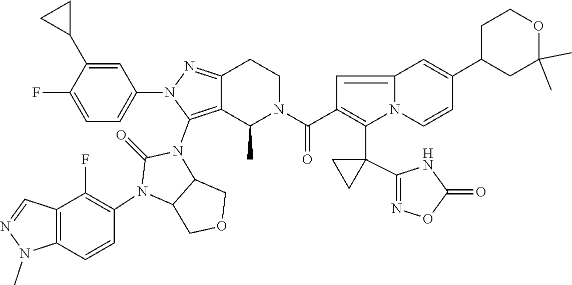 [SMILES: C[C@H]1c2c(nn(-c3ccc(F)c(C4CC4)c3)c2N2C(=O)N(c3ccc4c(cnn4C)c3F)C3COCC32)CCN1C(=O)c1cc2cc(C3CCOC(C)(C)C3)ccn2c1C1(c2noc(=O)[nH]2)CC1] | 1-((4S)-2-(3-cyclopropyl- 4-fluorophenyl)-5-(7-(2,2- dimethyltetrahydro-2H- pyran-4-yl)-3-(1-(5-oxo- 4,5-dihydro- 1,2,4-oxadiazol- 3-yl)cyclopropyl) indolizine-2-carbonyl)-4- methyl-4,5,6,7-tetrahydro- 2H-pyrazolo[4,3-c]pyridin- 3-yl)-3-(4-fluoro-1- methyl-1H-indazol-5- yl)tetrahydro-1H-furo[3,4- d]imidazol-2(3H)-one | // |
| 60 | 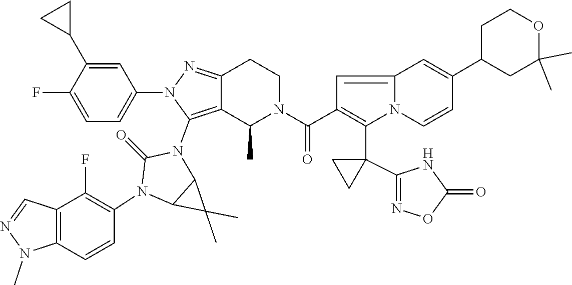 [SMILES: C[C@H]1c2c(nn(-c3ccc(F)c(C4CC4)c3)c2N2C(=O)N(c3ccc4c(cnn4C)c3F)C3C2C3(C)C)CCN1C(=O)c1cc2cc(C3CCOC(C)(C)C3)ccn2c1C1(c2noc(=O)[nH]2)CC1] | 3-(1-(2-((4S)-2-(3-cyclo- propyl-4-fluorophenyl)-3- (4-(4-fluoro-1-methyl- 1H-indazol-5-yl)-6,6- dimethyl-3-oxo-2,4-diaza- bicyclo[3.1.0]hexan-2-yl)- 4-methyl-4,5,6,7- tetrahydro- 2H-pyrazolo[4,3-c] pyridine-5-carbonyl)-7- (2,2-dimethyltetrahydro- 2H-pyran- 4-yl)indolizin-3- yl)cyclopropyl)-1,2,4- oxadiazol-5(4H)-one | // |
| 61 | 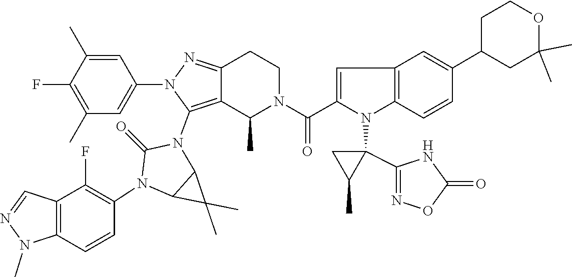 [SMILES: Cc1cc(-n2nc3c(c2N2C(=O)N(c4ccc5c(cnn5C)c4F)C4C2C4(C)C)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2- dimethyltetrahydro-2H- pyran-4-yl)-2- ((4S)-3-(4-(4-fluoro- 1-methyl-1H-indazol- 5-yl)-6,6-dimethyl-3- oxo-2,4-diazabicyclo [3.1.0]hexan- 2-yl)-2-(4-fluoro- 3,5-dimethylphenyl)-4- methyl-4,5,6,7-tetrahydro- 2H-pyrazolo[4,3-c] pyridine-5-carbonyl)- 1H-indol-1-yl)-2-methyl- cyclopropyl)-1,2,4- oxadiazol-5(4H)-one | // |
| 62 | 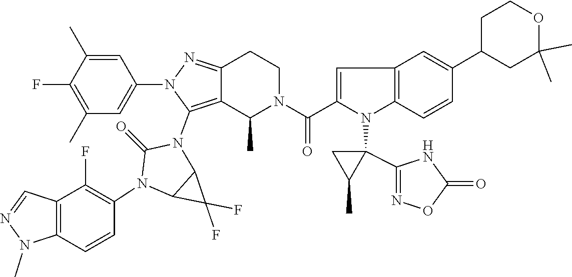 [SMILES: Cc1cc(-n2nc3c(c2N2C(=O)N(c4ccc5c(cnn5C)c4F)C4C2C4(F)F)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(2-((4S)- 3-(6,6-difluoro-4- (4-fluoro-1-methyl- 1H-indazol-5- yl)-3-oxo-2,4- diazabicyclo[3.1.0] hexan-2-yl)-2-(4- fluoro-3,5- dimethylphenyl)-4- methyl-4,5,6,7-tetrahydro- 2H-pyrazolo[4,3- c]pyridine-5-carbonyl)- 5-(2,2-dimethyltetrahydro- 2H-pyran-4-yl)-1H-indol- 1-yl)-2-methyl- cyclopropyl)-1,2,4- oxadiazol-5(4H)-one | // |
| 63 | 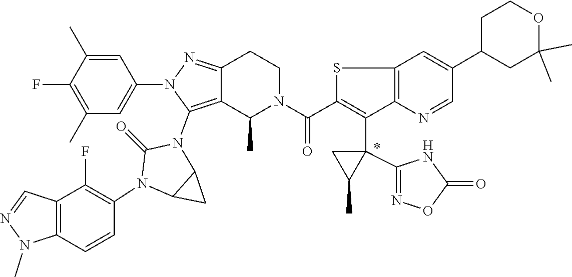 [SMILES: Cc1cc(-n2nc3c(c2N2C(=O)N(c4ccc5c(cnn5C)c4F)C4CC42)[C@H](C)N(C(=O)c2sc4cc(C5CCOC(C)(C)C5)cnc4c2C2(c4noc(=O)[nH]4)C[C@@H]2C)CC3)cc(C)c1F] | 3-((2S)-1-(6- (2,2-dimethyl- tetrahydro-2H-pyran-4- yl)-2-((4S)-3-(4-(4-fluoro- 1-methyl-1H-indazol-5- yl)-3-oxo-2,4-diazabicyclo [3.1.0]hexan-2-yl)-2-(4- fluoro-3,5- dimethylphenyl)- 4-methyl-4,5,6,7- tetrahydro-2H-pyrazolo [4,3-c]pyridine- 5-carbonyl)thieno [3,2-b]pyridin-3-yl)- 2-methylcyclopropyl)- 1,2,4-oxadiazol- 5(4H)-one | // |

*LC/MS data for the prepared compounds is obtained using the above indicated method.
The symbol “//” indicates the example can be prepared using appropriate intermediates, which can be readily synthesized by methods known in the art, and sequential modifications as necessary.

### Cell Proliferation Assays

Intracellular cAMP concentration was detected by TR-FRET method to evaluate the activity of drug-induced GLP1R receptor. The cells were prepared in an experimental buffer solution (HBSS, 20 mM HEPES (pH 7.5), 0.1% BSA, 500 μM IBMX) and inoculated into 384 cell culture plates with a inoculated density of 2000 cells and a inoculated volume of 15 μL per well. The compound was diluted by 3 times gradient with DMSO, 10 concentration gradients, and the 10 concentration gradients were diluted by 100 times in Buffer. The maximum concentration point of compound to be tested was 12 μM. The inoculated 384 cell plates were added with 5 μL prepared working solution and incubated at 37° C. for 30 min. Eu-cAMP tracer was diluted 50 times and Ulight-anti-cAMP 150 times by detection buffer (cAMP Kit, Perkin Elmer, cat #TRF0263). At first, 10 μL Eu-cAMP tracer was added to each experimental well, and then 10 μL Uligh-anti-cAMP was added to each experimental well. Centrifuged the assay plate at 200 g, RT, for 30 s, incubated it at 25° C. for 1 h. Collected the data by Envision 2105 microplate reader with a HTRF module. Excitation: 340 nm; emission: 665 nm and 615 nm.

The activation rate of each concentration of the tested compound to the positive drug was calculated by the formula % Activity=100 (Signal_{cmpd}Signal_{Ave_PC})/(Signal_{Ave_VC}Signal_{Ave_PC})×100, EC50 values and nonlinear regression curve fits were obtained using GraphPad Prism software.

TABLE 2

| EXAMPLE | cAMP Assay EC₅₀ (nM) |
|---|---|
| 2 | 0.511 |
| 3 | 0.406 |
| 6 | 0.087 |
| 29 | 0.093 |
| 30 | 0.044 |
| 31 | 0.182 |

1. A compound of formula (I): 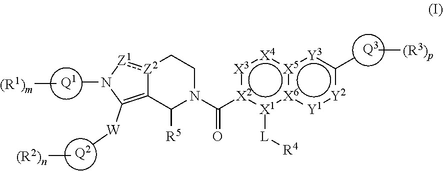
or a pharmaceutically acceptable salt thereof, wherein: [symbol]
is a double bond or a single bond;
X¹, X², X⁵ and X⁶ are independently selected from N and C;
X³ is selected from O, S, N, NR^{7.3} and CR^{7.3},
X⁴ is absent or selected from N, C(O) and CR^{7.4;}
Y¹ is selected from N and CR^{8.1;}
Y² is selected from N and CR^{8.2};
Y³ is selected from N and CR^{8.3;}
Z¹ is selected from N and C(O);
Z² is selected from N and C;
W is selected from 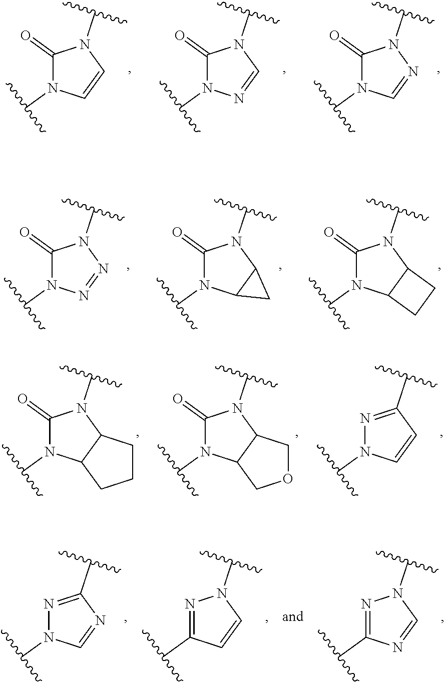
which are each unsubstituted or substituted with at least one substituent, independently selected from RX;
L is selected from —CR^{C0}R^{D0}(CR^{C0}R^{D0})ᵤ—, —(CR^{C0}R^{D0})ᵤO(CR^{C0}R^{D0})ₜ—, —(CR^{C0}R^{D0})ₜNR^{A0}(CR^{C0}R^{D0})ₜ— and —(CR^{C0}R^{D0})ᵤS(O)ᵣ(CR^{C0}R^{D0})ₜ—;
Q¹, Q² and Q³ is selected from C₃₋₁₀ cycloalkyl, heterocyclyl, aryl and heteroaryl;
each R¹, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═NR^{E1})R^{A1}, —C(═N—OR^{B1})R^{A1}, —C(O)OR^{A1}, —OC(O)R^{A1}, —C(O)NR^{A1}R^{B1}, —C(O)NR^{A1}S(O)ᵣR^{A1}, —C(O)NR^{A1}S(O)₂OR^{A1}, —C(O)NR^{A1}S(O)ᵣNR^{A1}R^{B1}, —C(O)NR^{A1}S(O)(═NR^{E1})R^{B1}, —C(O)NR^{A1}S(O)(═NR^{E1}) NR^{A1}R^{B1}, —NR^{A1}C(O)R^{B1}, —C(═NR^{E1}) NR^{A1}R^{B1}, —NR^{A1}C(═NR^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1}) NR^{A1}R^{B1}, —S(O)ᵣR^{A1}, —S(O)(═NR^{E1})R^{B1}, —N═S(O)R^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}, —S(O)ᵣNR^{A1}R^{B1}, —S(O)(═NR^{E1}) NR^{A1}R^{B1}, —NR^{A1}S(O)₂NR^{A1}R^{B1}, —NR^{A1}S(O)(═NR^{E1}) NR^{A1}R^{B1}, —P(O)R^{A1}R^{B1} and —P(O)(OR^{A1})(OR^{B1}), wherein alkyl, alkenyl, alkynyl, cycloalkyl, heterocyclyl, aryl and heteroaryl are each unsubstituted or substituted with at least one substituent, independently selected from R^{X1};
each R², at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —C(O)NR^{A2}S(O)(═NR^{E2})R^{B2}, —C(O)NR^{A2}S(O)(═NR^{E2}) NR^{A2}R^{B2}, —NR^{A2}C(O)R^{B2}, —C(═NR^{E2}) NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2}) NR^{A2}R^{B2}, —S(O)(═NR^{E2})R^{B2}, —N═S(O)R^{A2}R^{B2}, —NR^{A2}S(O)(═NR^{E2})R^{B2}, —S(O)(═NR^{E2}) NR^{A2}R^{B2} and —NR^{A2}S(O)(═NR^{E2}) NR^{A2}R^{B2}, wherein alkyl, alkenyl, alkynyl, cycloalkyl, heterocyclyl, aryl and heteroaryl are each unsubstituted or substituted with at least one substituent, independently selected from R^{X2};
each R³, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —C(O)NR^{A3}S(O)ᵣR^{A3}, —C(O)NR^{A3}S(O)₂OR^{A3}, —C(O)NR^{A3}S(O)ₜNR^{A3}R^{B3}, —C(O)NR^{A3}S(O)(═NR^{E3})R^{B3}, —C(O)NR^{A3}S(O)(═NR^{E3})NR^{A3}R^{B3}, —NR^{A3}C(O)R^{B3}, —C(═NR^{E3})NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3}) NR^{A3}R^{B3}, —S(O)ᵣR^{A3}, —S(O)(═NR^{E3})R^{B3}, —N═S(O)R^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3}) NR^{A3}R^{B3}, —NR^{A3} S(O)₂NR^{A3}R^{B3}, —NR^{A3}S(O)(═NR^{E3}) NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R^{X3};
R⁴ is selected from —C(O)OH, —C(O)NHS(O)ᵣR^{A4}, —(CR^{c1}R^{d1}) NHS(O)ᵣR^{A4}, heterocyclyl and heteroaryl;
R⁵ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A5}R^{B5}, —OR^{A5} and —C(O)R^{A5}, wherein alkyl, alkenyl, alkynyl, cycloalkyl and heterocyclyl are each unsubstituted or substituted with at least one substituent, independently selected from R^{X5};
each R⁶, at each occurrence, is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, CN, NO₂, —NR^{A6}R^{B6}, —OR^{A6} and —C(O)R^{A6}, wherein alkyl, alkenyl, alkynyl, cycloalkyl and heterocyclyl are each unsubstituted or substituted with at least one substituent, independently selected from R^{X6};
or “R⁵ and R⁶” or “two of R⁶” together with the atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X6},
R^{7.3} and R^{7.4} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A7}R^{B7}, —OR^{A7}, —C(O)R^{A7}, —C(═NR^{E7})R^{A7}, —C(═N—OR^{B7})R^{A7}, —C(O)OR^{A7}, —OC(O)R^{A7}, —C(O)NR^{A7}R^{B7}, —C(O)NR^{A7}S(O)ᵣR^{A7}, —C(O)NR^{A7}S(O)₂OR^{A7}, —C(O)NR^{A7}S(O)ᵣNR^{A7}R^{B7}, —C(O)NR^{A7}S(O)(═NR^{E7})R^{B7}, —C(O)NR^{A7}S(O)(═NR^{E7}) NR^{A7}R^{B7}, —NR^{A7}C(O)R^{B7}, —C(═NR^{E7}) NR^{A7}R^{B7}, —NR^{A7}C(═NR^{E7})R^{B7}, —OC(O)NR^{A7}R^{B7}, —NR^{A7}C(O)OR^{B7}, —NR^{A7}C(O)NR^{A7}R^{B7}, —NR^{A7}C(S)NR^{A7}R^{B7}, —NR^{A7}C(═NR^{E7}) NR^{A7}R^{B7}, —S(O)ᵣR^{A7}, —S(O)(═NR^{E7})R^{B7}, —N═S(O)R^{A7}R^{B7}, —S(O)₂OR^{A7}, —OS(O)₂R^{A7}, —NR^{A7}S(O)ᵣR^{B7}, —NR⁴⁷S(O)(═NR^{E7})R^{B7}, —S(O)ᵣNR^{A7}R^{B7}, —S(O)(═NR^{E7}) NR 47R^{B7}, —NR^{A7}S(O)₂NR^{A7}R^{B7}, —NR^{A7}S(O)(═NR^{E7}) NR^{A7}R^{B7}, —P(O)R^{A7}R^{B7} and —P(O)(OR^{A7})(OR^{B7}), wherein alkyl, alkenyl, alkynyl, cycloalkyl, heterocyclyl, aryl and heteroaryl are each unsubstituted or substituted with at least one substituent, independently selected from R^{X7};
R^{8.1}, R^{8.2} and R^{8.3} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁-4 alkyl, aryl, aryl-C₁₋₄ alkyl, heteroaryl, heteroaryl-C₁₋₄ alkyl, CN, NO₂, —NR^{A8}R^{B8}, —OR^{A8}, —C(O)R^{A8}, —C(═NR^{E8})R^{A8}, —C(═N—OR^{B8})R^{A8}, —C(O)OR^{A8}, —OC(O)R^{A8}, —C(O)NR^{A8}R^{B8}, —C(O)NR^{A8}S(O)ᵣR^{A8}, —C(O)NR^{A8}S(O)₂OR^{A8}, —C(O)NR^{A8}S(O)ᵣNR^{A8}R^{B8}, —C(O)NR^{A8}S(O)(═NR^{E8})R^{B8}, —C(O)NR^{A8}S(O)(═NR^{E8}) NR^{A8}R^{B8}, —NR^{A8}C(O)R^{B8}, —C(═NR^{E8}) NR^{A8}R^{B8}, —NR^{A8}C(═NR^{E8})R^{B8}, —OC(O)NR^{A8}R^{B8}, —NR^{A8}C(O)OR^{B8}, —NR^{A8}C(O)NR^{A8}R^{B8}, —NR^{A8}C(S)NR^{A8}R^{B8}, —NR^{A8}C(═NR^{E8}) NR^{A8}R^{B8}, —S(O)ᵣR^{A8}, —S(O)(═NR^{E8})R^{B8}, —N═S(O)R^{A8}R^{B8}, —S(O)₂OR^{A8}, —OS(O)₂R^{A8}, —NR^{A8}S(O)ᵣR^{B8}, —NR^{A8}S(O)(═NR^{E8})R^{B8}, —S(O)ᵣNR^{A8}R^{B8}, —S(O)(═NR^{E8}) NR^{A8}R^{B8}, —NR^{A8}S(O)₂NR^{A8}R^{B8}, —NR^{A8}S(O) (—NR^{E8}) NR^{A8}R^{B8}, —P(O)R^{A8}R^{B8} and —P(O)(OR^{A8})(OR^{B8}), wherein alkyl, alkenyl, alkynyl, cycloalkyl, heterocyclyl, aryl and heteroaryl are each unsubstituted or substituted with at least one substituent, independently selected from R^{X8};
R^{A0} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
each R^{A1} and RBI are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X1};
or each “R^{A1} and R^{B1}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X1} groups;
each R^{A2} and R^{B2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X2};
or each “R^{A2} and R^{B2}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X2} groups;
each R^{A3} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X3};
or each “R^{A3} and R^{B3}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X3} groups;
R^{A4} is selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X4};
each R^{A5} and R^{B5} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X5},
or each “R^{A5} and R^{B5}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X5} groups;
each R^{A6} and R^{B6} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X6},
or each “R^{A6} and R^{B6}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X6} groups;
each R^{A7} and R^{B7} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X7};
or each “R^{A7} and R^{B7}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X7} groups;
each R^{A8} and R^{B8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X8},
or each “R^{A8} and R^{B8}” together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{X8} groups;
each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X0};
or R^{C0} and R^{D0} together with the carbon atom(s) to which they are attached form a C₃₋₁₀ cycloalkyl or heterocyclic ring of 4 to 12 members containing 1, 2 or 3 heteroatoms independently selected from oxygen, sulfur, nitrogen and phosphorus, wherein cycloalkyl and heterocyclic ring are each unsubstituted or substituted with at least one substituent, independently selected from R^{X0:}
each R^{E1}, R^{E2}, R^{E3}, R^{E7} and R^{E8} are independently selected from hydrogen, C₁₋₁₀ alkyl, CN, NO₂, —OR^{a1}, —SR^{a1}, —S(O)ᵣRal, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from R^{X};
each R^{X}, R^{X0}, R^{X1}, R^{X2}, R^{X3}, R^{X4}, R^{X5}, R^{X6}, R^{X7} and R^{X8} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1}, —(CR^{c1}R^{d1})ₜC(═N—OR^{b1})R^{a1}, —(CR^{e1}R^{d1})C(O)OR^{b1}, —(CR^{c1}R^{d1})ₜC(O)R^{b1}, —(CR^{c1}R^{d1})ₜC(O)NR^{a1}R^{b1}, —(CR^{c1}R^{d1})ₜNR^{a1}C(O)R^{b1}, —(CR^{c1}R^{d1})ₜC(═NR^{e1}) NR^{a1}R^{b1}, —(CR^{c1}R^{d1})ₜNR^{a1}C(═NR^{e1})R^{b1}, —(CR^{c1}R^{d1})ₜC(O)NR^{a1}R^{b1}, —(CR^{c1}R^{d1})ₜNR^{a1}C(O)OR^{b1}, —(CR^{c1}R^{d1})ₜNR^{a1}C(O)NR^{a1}R^{b1}, —(CR^{c1}R^{d1}) NR^{a1}C(S)NR^{a1}R^{b1}, —(CR^{c1}R^{d1})ₜNR^{a1}C(═NR^{e1}) NR^{a1}R^{b1}, —(CR^{c1}R^{d1})S(O)ᵣR^{b1}, —(CR^{c1}R^{d1})ₜS(O)(═NR^{e1})R^{b1}, —(CR^{c1}R^{d1})ₜN═S(O)R^{a1}R^{b1}, —(CR^{c1}R^{d1})ₜS(O)₂OR^{b1}, —(CR^{c1}R^{d1})ₜS(O)₂R^{b1}, —(CR^{c1}R^{d1})ₜNR^{a1}S(O)ᵣR^{b1}, —(CR^{c1}R^{d1})ₜNR^{a1}S(O)(═NR^{e1})R^{b1}, —(CR^{c1}R^{d1})ₜS(O)ᵣNR^{a1}R^{b1}, —(CR^{c1}R^{d1})ₜS(O)(═NR^{e1}) NR^{a1}R^{b1}, —(CR^{c1}R^{d1})ₜNR^{a1}S(O)₂NR^{a1}R^{b1}, —(CR^{c1}R^{d1})ₜNR^{a1}S(O)(═NR^{e1}) NR^{a1}R^{b1}, —(CR^{c1}R^{d1})ₜP(O)R^{a1}R^{b1} and —(CR^{c1}R^{d1})ₜP(O)(OR^{a1})(OR^{b1}), wherein alkyl, alkenyl, alkynyl, cycloalkyl, heterocyclyl, aryl and heteroaryl are each unsubstituted or substituted with at least one substituent, independently selected from RY;
each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from RY;
or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 RY groups;
each R^{e1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from RY;
or R^{e1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 RY groups;
each R^{e1} is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR^{a2}R^{b2} and —C(O)NR^{a2}R^{b2};
each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})ₜNR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a2}, —(CR^{c2}R^{d2})ₜC(═N—OR^{b2})R^{a2}, —(CR^{c2}R^{d2})ₜC(O)OR^{b2}, —(CR^{c2}R^{d2})ₜC(O)R^{b2}, —(CR^{c2}R^{d2})ₜC(O)NR^{a2}R^{b2}, —(CR^{c2}R^{d2}), NR^{a2}C(O)R^{b2}, (CR^{c2}R^{d2})ₜC(═NR^{e2})NR^{a2}R^{b2}, —(CR^{c2}R^{d2}), NR^{a2}C(═NR^{e2})R^{b2}, —(CR^{c2}R^{d2}), OC(O)NR^{a2}R^{b2}, —(CR^{c2}R^{d2})ₜNR^{a2}C(O)OR^{b2}, —(CR^{c2}R^{d2})ₜNR^{a2}C(O)NR^{a2}R^{b2}, —(CR^{c2}R^{d2}), NR^{a2}C(S)NR^{b2}R^{b2}, —(CR^{c2}R^{d2})ₜNR^{a2}C(═NR^{e2})NR^{a2}R^{b2}, —(CR^{c2}R^{d2})ₜS(O)ᵣR^{b2}, —(CR^{c2}R^{d2}); S(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2}), OS(O)₂R^{b2}, —(CR^{c2}R^{d2})ₜNR^{a2}S(O), R^{b2}, —(CR^{c2}R^{d2})ₜNR^{a2}S(O)(═NR^{e2})R^{b2}, —(CR^{c2}R^{d2})S(O)ᵣNR^{a2}R^{b2}, —(CR^{c2}R^{d2})ₜS(O)(═NR^{e2})NR^{a2}R^{b2}, —(CR^{c2}R^{d2})ₜNR^{a2}S(O)₂NR^{a2}R^{b2}, —(CR^{c2}R^{d2})ₜNR^{a2}S(O)(═NR^{e2})NR^{a2}R^{b2}, —(CR^{c2}R^{d2})ₜP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;
each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{a2} and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{e2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{c2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O) C₁₋₄ alkyl, —C(O) C₃₋₁₀ cycloalkyl, —C(O)OC₁₋₄ alkyl, —C(O)OC₃₋₁₀ cycloalkyl, —C(O)N(C₁₋₄ alkyl)₂, —C(O)N(C₃₋₁₀ cycloalkyl)₂, —S(O)₂C₁₋₄ alkyl, —S(O)₂C₃₋₁₀ cycloalkyl, —S(O)₂N(C₁₋₄ alkyl)₂ and —S(O)₂N(C₃₋₁₀ cycloalkyl)₂;
m, n, p and q are independently selected from 0, 1, 2, 3 and 4;
each r is independently selected from 0, 1 and 2;
each tis independently selected from 0, 1, 2, 3 and 4;
each u is independently selected from 0, 1, 2, 3 and 4,
provided that when Z¹ is N, X² is C, X³ is CR^{7.3} or N, X⁴ is absent, R⁵ and R⁶ or any two of R⁶ do not form any ring, W is 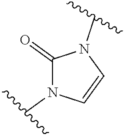 [SMILES: *n1ccn(*)c1=O]
and L is selected from —CR^{C0}R^{D0} (CR^{C0}R^{D0})ᵤ—, then at least one of R^{C0} and R^{D0} is substituted with R^{X0}, and R^{X0} is selected from CN, C₂₋₁₀ alkenyl and C₂₋₁₀ alkynyl.

2. (canceled)

3. (canceled)

4. (canceled)

5. (canceled)

6. (canceled)

7. A compound of claim 1 or a pharmaceutically acceptable salt thereof, wherein R⁵ is selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH.

8. (canceled)

9. A compound of claim 1 or a pharmaceutically acceptable salt thereof, wherein each R⁶, at each occurrence, is selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH.

10. (canceled)

11. A compound of claim 1 or a pharmaceutically acceptable salt thereof, wherein “R⁵ and R⁶” or “two of R⁶” together with the atom(s) to which they are attached form a C₃₋₈ cycloalkyl or heterocyclic ring of 4 to 8 members containing 1, 2 or 3 heteroatoms independently selected from oxygen, sulfur and nitrogen, wherein cycloalkyl and heterocyclic ring are each unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1.}

12. (canceled)

13. (canceled)

14. (canceled)

15. A compound of claim 1 or a pharmaceutically acceptable salt thereof, wherein Q² is selected from

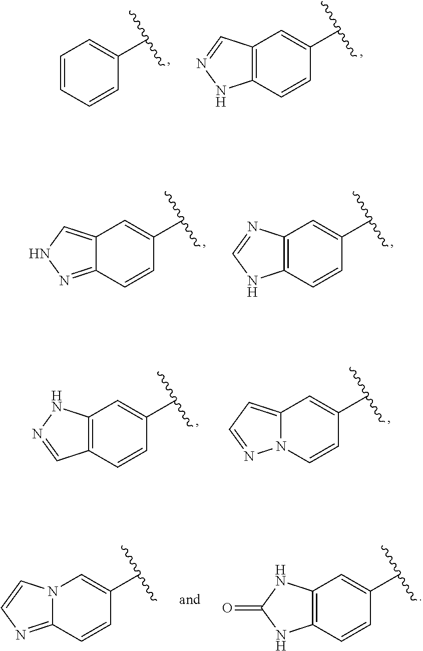

16. (canceled)

17. (canceled)

18. A compound of claim 1 or a pharmaceutically acceptable salt thereof, wherein each R², at each occurrence, is independently selected from hydrogen, methyl, ethyl, isopropyl, cyclopropyl, pyran, methoxy, ethoxy, F, Cl, Br, —CN, —NO₂, —NH₂, —NHCH₃, —OH and 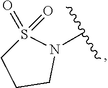 [SMILES: *N1CCCS1(=O)=O]
wherein methyl, ethyl, isopropyl, cyclopropyl, pyran, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{c1}R^{d1})ₜOR^{b1}.

19. (canceled)

20. A compound of claim 1 or a pharmaceutically acceptable salt thereof, wherein Q¹ is selected from phenyl, pyridinyl and pyrimidinyl.

21. (canceled)

22. (canceled)

23. A compound of claim 1 or a pharmaceutically acceptable salt thereof, wherein each R¹, at each occurrence, is independently selected from hydrogen, methyl, ethyl, isopropyl, cyclopropyl, methoxy, ethoxy, F, Cl, Br, —CN, —NO₂, —NH₂ and —OH, wherein methyl, ethyl, isopropyl, cyclopropyl, methoxy and ethoxy are unsubstituted or substituted with at least one substituent, independently selected from C₁₋₁₀ alkyl, C₃₋₁₀ cycloalkyl, halogen, CN, NO₂, —(CR^{e1}R^{d1})ₜNR^{a1}R^{b1} and —(CR^{c1}R^{d1})ₜOR^{b1.}

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A compound of claim 1 or a pharmaceutically acceptable salt thereof, wherein R^{7.3} and R^{7.4} are independently selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH.

40. (canceled)

41. (canceled)

42. (canceled)

43. (canceled)

44. (canceled)

45. (canceled)

46. (canceled)

47. A compound of claim 1 or a pharmaceutically acceptable salt thereof, wherein R^{8.1}, R^{8.2} and R^{8.3} are independently selected from hydrogen, methyl, difluoromethyl, trifluoromethyl, ethyl, difluoroethyl, trifluoroethyl, isopropyl, cyclopropyl, methoxy, ethoxy, F, Cl, Br, —CN, —NO₂, —NH₂ and —OH.

48. (canceled)

49. (canceled)

50. A compound of claim 1 or a pharmaceutically acceptable salt thereof, wherein Z¹ is N, X² is C, X³ is CR^{7.3} or N, X⁴ is absent, R⁵ and R⁶ or any two of R⁶ do not form any ring, W is 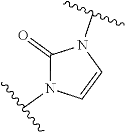 [SMILES: *n1ccn(*)c1=O]
and L is selected from —CR^{C0}R^{D0}(CR^{C0}R^{D0})ᵤ—, at least one of R^{C0} and R^{D0} is substituted with R^{X0}, and R^{X0} is selected from CN, vinyl and ethynyl.

51. A compound of claim 1 or a pharmaceutically acceptable salt thereof, wherein each R^{C0} and R^{D0} are independently selected from hydrogen, methyl, ethyl, isopropyl, cyclopropyl, methoxy and ethoxy, wherein methyl, ethyl, isopropyl, cyclopropyl, methoxy and ethoxy are unsubstituted or substituted with at least one substituent, independently selected from R^{X0};
or R^{C0} and R^{D0} together with the carbon atom(s) to which they are attached form C₃₋₁₀ cycloalkyl, wherein cycloalkyl unsubstituted or substituted with at least one substituent, independently selected from R^{X0}.

52. (canceled)

53. A compound of claim 1 or a pharmaceutically acceptable salt thereof, wherein R^{X0} is selected from hydrogen, methyl, difluoromethyl, trifluoromethyl, ethyl, difluoroethyl, trifluoroethyl, isopropyl, cyclopropyl, methoxy, ethoxy, vinyl, ethynyl, F, Cl, Br, —CN, —NO₂, —NH₂ and —OH.

54. (canceled)

55. (canceled)

56. A compound of claim 1 or a pharmaceutically acceptable salt thereof, wherein R⁴ is selected from

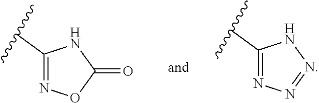 [SMILES: *c1noc(=O)[nH]1 ; *c1nnn[nH]1]

57. (canceled)

58. A compound of claim 1 or a pharmaceutically acceptable salt thereof, wherein Q³ is selected from

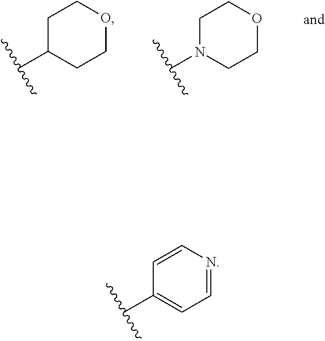 [SMILES: *C1CCOCC1 ; *N1CCOCC1 ; *c1ccncc1]

59. (canceled)

60. (canceled)

61. A compound of claim 1 or a pharmaceutically acceptable salt thereof, wherein each R³, at each occurrence, is independently selected from hydrogen, methyl, ethyl, isopropyl, cyclopropyl, methoxy, ethoxy, F, Cl, Br, —CN, —NO₂, —NH₂ and —OH, wherein methyl, ethyl, isopropyl, cyclopropyl, methoxy and ethoxy are unsubstituted or substituted with at least one substituent, independently selected from C₁₋₁₀ alkyl, C₃₋₁₀ cycloalkyl, halogen, CN, NO₂, —(CR^{c1}R^{d1})ₜNR^{a1}R^{b1} and —(CR^{e1}R^{d1})ₜOR^{b1}.

62. A compound of claim 1 or a pharmaceutically acceptable salt thereof, wherein the compound is selected from

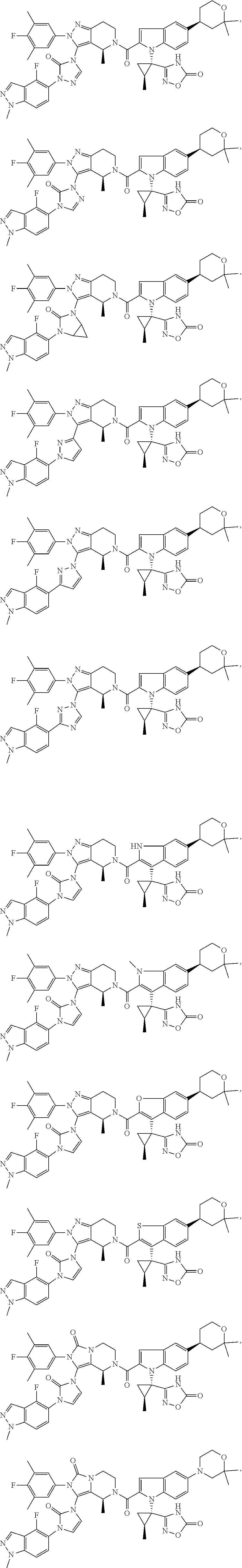

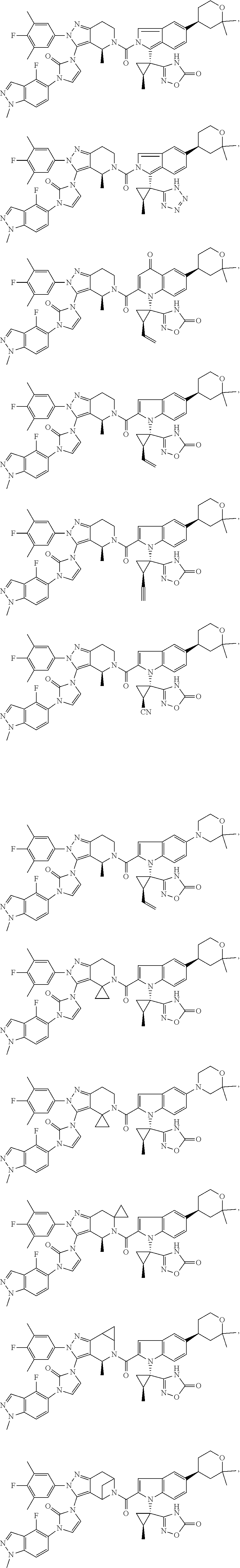

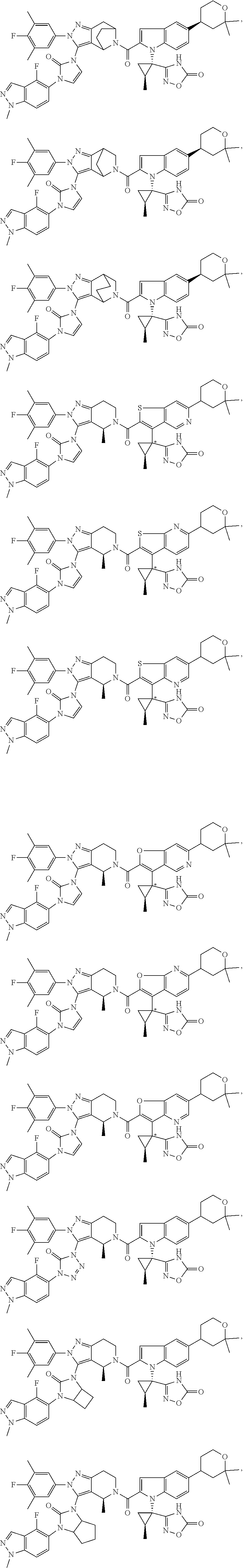

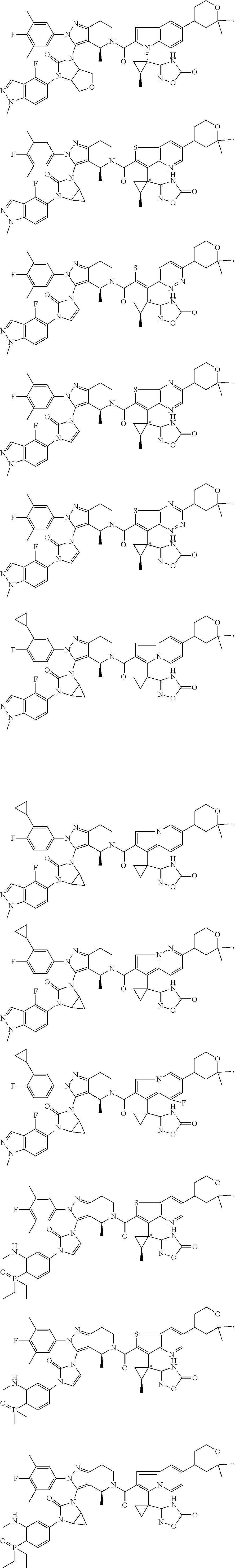

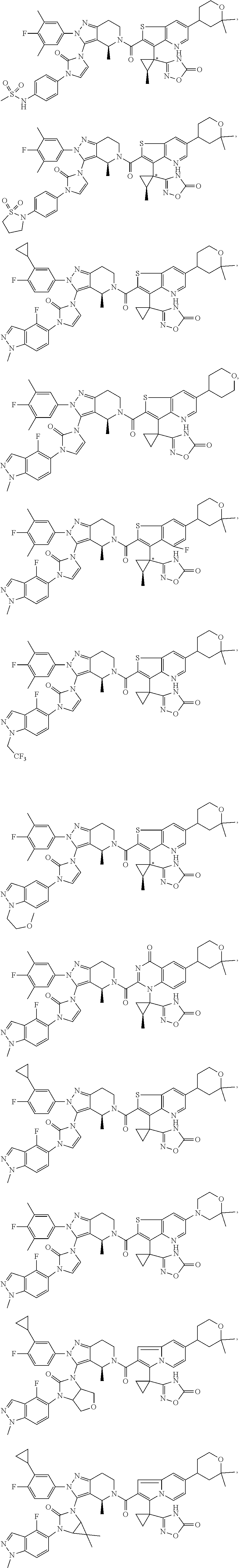

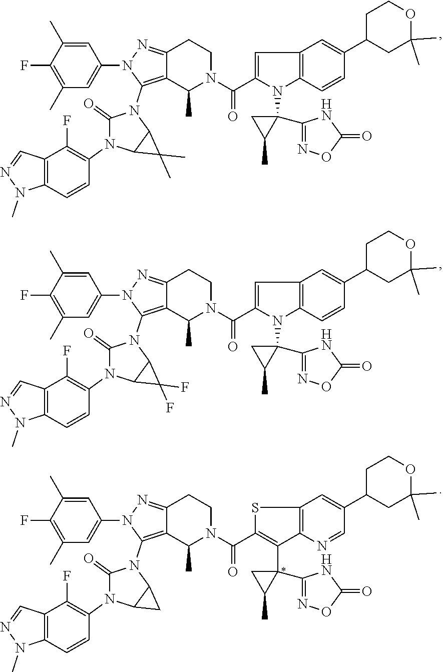 [SMILES: Cc1cc(-n2nc3c(c2N2C(=O)N(c4ccc5c(cnn5C)c4F)C4C2C4(C)C)[C@H](C)N(C(=O)c2cc4cc(C5CCOC(C)(C)C5)ccc4n2[C@@]2(c4noc(=O)[nH]4)C[C@@H]2C)CC3)cc(C)c1F ; Cc1cc(-n2nc3c(c2N2C(=O)N(c4ccc5c(cnn5C)c4F)C4C2C4(F)F)[C@H](C)N(C(=O)c2cc4cc(C5CCOC(C)(C)C5)ccc4n2[C@@]2(c4noc(=O)[nH]4)C[C@@H]2C)CC3)cc(C)c1F ; Cc1cc(-n2nc3c(c2N2C(=O)N(c4ccc5c(cnn5C)c4F)C4CC42)[C@H](C)N(C(=O)c2sc4cc(C5CCOC(C)(C)C5)cnc4c2C2(c4noc(=O)[nH]4)C[C@@H]2C)CC3)cc(C)c1F]

63. A pharmaceutical composition, comprising a compound of claim 1 or a pharmaceutically acceptable salt thereof and at least one pharmaceutically acceptable carrier.

64. (canceled)

65. A method of treating or preventing a GLP-1 receptor-mediated disease or disorder or of modulating GLP-1 receptor, comprising administering to a subject in need of such treatment an effective amount of a compound of claim 1 or a pharmaceutically acceptable salt thereof.

66. The method according to claim 65, wherein the disease or disorder is selected from diabetes, diabetic complication, obesity, impaired glucose tolerance, overweight condition, hyperlipidemia, hypercholesteremia, atherosclerosis, hypertension, coronary heart disease, congestive heart failure, cardiac arrhythmias, brain infarction, stroke, liver disease, nonalcoholic fatty liver disease (NAFLD), nonalcoholic steatohepatitis (NASH), dementia, Parkinson's disease, and diabetic kidney disease.
